(19) United States
(12) Patent Application Publication
(10) Pub. No.: US 2024/0269320 A1
(43) Pub. Date: Aug. 15, 2024

(54) EXTENDED RELEASE DRUG DELIVERY SYSTEM FOR OCULAR DRUGS AND METHODS OF USE

(71) Applicant: Eyedea Bio, LLC, Chapel Hill, NC (US)

(72) Inventors: Andres GARCIA, Chapel Hill, NC (US); Xunpei LIU, Chapel Hill, NC (US)

(21) Appl. No.: 18/566,629

(22) PCT Filed: Jun. 1, 2022
(86) PCT No.: PCT/US2022/031777

§ 371 Date: Dec. 1, 2023

Related U.S. Application Data
(60) Provisional application No. 63/195,697, filed on Jun. 1, 2021.
(60) Provisional application No. 63/281,052, filed on Nov. 18, 2021.

(51) Int. Cl. A61K 47/69; A61K 9/00; A61K 47/65

(52) U.S. Cl. CPC A61K 47/6951; A61K 9/0048; A61K 47/65; A61K 47/6905; A61K 47/6949

(57) ABSTRACT

Described herein are therapeutic compositions and methods of use, for the delivery of various drug substances, in and around the eye, comprising: a drug substance, noncovalently interacting with one or more complexation agent particulates to form drug substance-complex particulates, admixed within a hydrophobic dispersal medium, that collectively forms a stable multiphasic colloidal suspension, that serves as an extended release drug delivery system for ocular drug delivery. Formulation of the drug substance in the multiphasic colloidal suspension can be administered in and around the eye to produce sustained release of therapeutic levels of drug substance within ocular tissues for one or more months without requiring retreatment.

### CLAIM OF PRIORITY

This patent application claims priority to U.S. provisional patent application No. 63/195,697, titled “INTRAVITREAL MITOCHONDRIAL-TARGETED PEPTIDE PRODRUGS AND METHODS OF USE”, filed on Jun. 1, 2021, and to U.S. provisional patent application No. 63/281,052, titled “INTRAVITREAL CORTICOSTEROID EXTENDED RELEASE IMPLANT AND METHODS OF USE,” filed on Nov. 18, 2021, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Extended release drug delivery systems (XRDDS) include devices, compositions, formulations or other systems used in the design, manufacture and administration of specific drug substances in a manner that regulates the drug release kinetics optimized for a specific therapeutic goal for a particular route of administration.

While several XRDDS have been developed and are employed for drug delivery in and around the eye, these systems have limitations, including a limited compatibility with only certain types of drug substances, rapid and excessive release (i.e., “dump”) of drug payload, suboptimal rate or amount (i.e., insufficient or subtherapeutic drug release), or suboptimal duration of drug release (i.e., duration that is too short or too long).

Currently available ocular XRDDS have limited ability to customize specific kinetic drug release profile. The majority offer zero-order (i.e., linear) release kinetics, while others (particularly those with rapid excessive release) may have a brief linear release, followed by a brief excessive recessive release, or “dump,” of all remaining drug substance. However, there is a need for XRDDS that are customizable and that can provide different kinetics of release, including two-phase release kinetics and three-phase release kinetics. For example, many ocular diseases, such as wet age-related macular degeneration (AMD) and diabetic macular edema (DME). require an initial higher dose of drug to treat and reverse existing disease manifestations, followed by a lower dose of drug to prevent disease recurrence, making these diseases ideal for two-phase release kinetics.

A number of ocular diseases, including several diseases of the retina, such as dry age related macular degeneration (AMD), as well as diseases of the optic nerve, uveal tract, and anterior segment of the eye, have limited or no effective therapy, due in part to a lack of effective and versatile technologies for sustained-release drug delivery for the eye.

In diseases such as wet AMD, diabetic retinopathy, diabetic macular edema (DME), and retinal vein occlusion (RVO), while effective therapies in the form of intravitreal anti-VEGF drugs and corticosteroids are available, these drugs must be administered every 1-2 months in order to achieve optimal treatment outcomes. This presents a significant treatment burden for affected patients and their caregivers, and for treating physicians. This ultimately leads to undertreatment or inefficient treatment and consequently, suboptimal vision (i.e., limited vision gains or vision loss) for a high percentage of affected patients, in spite of the efficacy of available treatments.

In diseases such as glaucoma, topical eyedrop drugs are effective for lowering intraocular pressure and reduce risk of associated vision loss. However, lack of patient compliance with self-administration and intermittent dosing results in undertreatment and suboptimal outcomes for affected patients.

Thus, it would be highly desirable to provide new and effective versatile technologies for sustained release of drug delivery which are compatible with a variety of drug types, including small molecules, antibodies, biologics, large protein, peptides, and which are customizable to achieve desired kinetics and duration of drug release to achieve therapeutic benefit.

### SUMMARY OF THE DISCLOSURE

Extended release drug delivery systems (XRDDS) include compositions, formulations, devices and/or systems used in the design, manufacture and administration of specific drug substances in a manner that regulates the drug release kinetics optimized for a specific therapeutic goal and for a particular route of administration.

Described herein are compositions of matter and methods of making and of use, for versatile extended release drug delivery systems (XRDDS), for the delivery of various drug substances, in and around the eye, comprising: a drug substance, noncovalently interacting with one or more complexation agent particulates to form drug substance-complex particulates, admixed within a hydrophobic dispersal medium, that collectively forms a stable multiphasic colloidal suspension (FIG. 1).

Herein, drug substance may include various small polypeptides, proteins, aptamers, other nucleic acid drugs, hydrophobic chemicals, hydrophilic chemicals, and other chemical compounds used for therapeutic purposes, that are capable of directly forming noncovalent complexes to one of six classes of complexation agents: fatty acid, organic compounds that can form keto-enol tautomers, charged phospholipid, charged protein, ribonucleic acid, and polysaccharide; and a prodrug of any active pharmaceutical ingredient (API) linked via cleavable covalent bond to a conjugation moiety, wherein the conjugation moiety forms complexes with one of six classes of complexation agents: fatty acid, organic compounds that can form keto-enol tautomers, charged phospholipid, charged protein, ribonucleic acid, and polysaccharide.

A conjugation moiety may be any chemical substance that can be covalently bound to an API. Certain conjugation moieties can be chosen for their ability to provide properties that the native API does not demonstrate, especially the ability to form reversible noncovalent complexes with complexation agents.

A complex is defined as a noncovalent interaction between the drug substance and a complexation agent.

A complexation agent is defined as: a chemical substance formulated as an irregularly shaped particulate ranging in size from 1 nanometer (nm) to 1000 micrometers (m); demonstrates a measurable binding capacity of selected drug substance, defined as a quantity of drug substance bound to a known quantity of complexation agent; demonstrates reversibility of drug binding, defined as a measurable unbound-bound ratio, or Kd, within a specific dispersal medium; and is a chemical substance not previously known or expected to form complexes with the selected drug substance. Binding of drug substance to a complexation agent, either directly or in a prodrug via the conjugation moiety, results in formation of drug substance-complex particulate. Certain well known chemical substances, including additives and excipients utilized in pharmaceutical industry, when formulated as irregular particulates, demonstrate a previously unknown and unexpected property to serve as complexation agents for various drug substances. These include six classes of chemical substances, that, when formulated as irregularly shaped particulates, are not previously known to serve as complexation agents for various drug substances: fatty acid, organic compounds that can form keto-enol tautomers, charged phospholipid, charged protein, ribonucleic acid, and polysaccharide.

As used herein, a particular complexation agent for a drug substance includes irregular particulate formulations, as opposed to dissolved individual molecules, of magnesium stearate, lecithin, albumin, cyclodextrin, and others, which represents a property not previously known or expected.

A dispersal medium is a vehicle utilized in colloid mixtures. Herein, a dispersal medium is defined as a hydrophobic, viscous oil, selected from among the four classes saturated fatty acid methyl esters, unsaturated fatty acid methyl esters, saturated fatty acid ethyl esters, or unsaturated fatty acid ethyl esters, that when admixed with drug substance-complex particulates, can form the drug substance multiphasic colloidal suspension, and is not previously known to form a multiphasic colloidal suspension with selected drug substance and the chosen complexation agents.

A colloidal suspension includes formulations that are viscous, flowable injectable liquids that may form a stable dispersal of particulates without migration or settling of the particulates (i.e., a colloid mixture).

Multiphasic colloidal suspension containing refers to a colloidal suspension in which the drug substance is present in at least two phases: free, unbound drug substance and drug substance bound to complexation agents (as well as less importantly, drug-drug aggregates). The drug substance-complex particulate serves a reservoir for drug substance when the particulate is admixed into the dispersal medium.

Thus, a drug substance multiphasic colloidal suspension may include a viscous, flowable injectable liquid that results in stably dispersed drug substance-complex particulates without migration or settling, may enable free drug substance to dissociate from the drug substance-complex particulates to create a free drug substance concentration in the dispersal medium; and the drug substance can freely diffuse through the multiphasic colloidal suspension system to exit the implant into the adjacent ocular physiologic environment. When the drug substance is a prodrug, on exposure of the prodrug to the ocular physiologic environment, the covalent bond linking the conjugation moiety is cleaved, releasing free API.

The drug substance multiphasic colloidal suspension enables a drug delivery system because the particulates are a reservoir of bound drug substance, each with a unique binding capacity and Kd (unbound-bound ratio), which in turn determines the composite amount of free drug substance in the dispersal medium. Knowledge of the Kd and the binding capacity of each drug substance-complex particulate can be used to calculate the total amount of free drug substance in the system, which in turn determines the rate and amount of release (FIG. 2). The relative ratio and amounts of different drug substance-complex particulates can be adjusted in a manner to create a calculatable unbound free drug substance within the system. The dynamic change of unbound, free drug substance within the system over the life of the implant is determined by the binding capacity and Kd of the drug substance-complex particulates within the drug substance multiphasic colloidal suspension.

In the methods and compositions described herein, the drug substance multiphasic colloidal suspension is injectable through a 20-gauge through 30-gauge size needle (depending on utilization) and provides stable dispersion of particulates without migration or settling when exposed to an ocular physiologic environment for the duration of the implant's lifetime (1 to 12 months). An ocular physiologic environment is defined as in vitro conditions with phosphate buffered saline (or comparable aqueous solvent) at 37° C. containing enzymes and proteins normally found in vitreous (representing injection into the vitreous) or with phosphate buffered saline at 37° C. containing plasma (representing injection into various periocular tissues). Alternatively, ocular physiologic environment may represent injection of the implant in vivo into the vitreous or into periocular tissues.

The drug substance multiphasic colloidal suspension also manifests the property of biodegradability when exposed to an ocular physiologic environment wherein biodegradability occurs by dissolution of the dispersal medium. The rate of biodegradation is proportional to the degree of solubility of the dispersal medium in the ocular physiologic environment. A dispersal medium with higher solubility will enable faster biodegradation of the multiphasic colloidal suspension when exposed to an ocular physiologic environment, while a dispersal medium with lower solubility will enable slower biodegradation of the multiphasic colloidal suspension when exposed to an ocular physiologic environment. This property of the drug substance multiphasic colloidal suspension can be used along with the volume of injected implant to determine durability of the implant in an ocular physiologic environment.

Formulation of the drug substance in the multiphasic colloidal suspension (FIG. 1), termed the implant, can be administered in and around the eye, i.e., into the vitreous humor, into the aqueous humor, into the suprachoroidal space, under the retina, under the conjunctiva, beneath Tenon's capsule, into orbital tissue, to produce sustained release of therapeutic levels of drug substance, for desired kinetics of release (FIG. 3) within ocular tissues for desired duration (1 to 12 months), for the treatment of various diseases and disorders.

The extended release drug delivery system (XRDDS) described herein is comprised of drug substance admixed with one or more particulate complexation agents to form “drug-complex” particulates, which are combined and dispersed within a selected dispersal medium to form a stable multiphasic colloidal suspension (FIG. 1).

Colloids are mixtures in which particulate substances are stably dispersed within a vehicle, called a dispersal medium, but do not settle or migrate. This differentiates a colloid from a suspension in which the particles settle within the suspension vehicle due to gravity. Typical particulate size for colloids is in the nanometer range. In colloids, the defining characteristic of the mixture is that particulates remain stably dispersed with minimal settling or migration. Colloid mixture in which particulates are dispersed in a liquid is called a “sol.” Colloid mixtures in which particulates are dispersed in a solid or semisolid is called a “solid colloid.” Colloid mixtures in which particulates are stably dispersed in a viscous semi-solid or solid dispersal medium have not been given a defined named. Herein, we refer to stably dispersed particulates as “colloidal suspension.” In the methods and compositions described herein, the dispersal medium may be a hydrophobic dispersal medium that facilitates a stable colloidal suspension. A drug substance multiphasic colloidal suspension is a suspension in which the drug substance is present in more than one phase, including free drug, drug-drug aggregates, and most importantly, drug noncovalently bound to complexation agent particulates.

Complexation occurs in two physicochemical circumstances. In one case, complexation occurs with noncovalent interactions between individual molecules (e.g., receptor-ligand interactions). This type of complexation is termed molecular complexation is not contemplated in the current composition.

The second circumstance involves a molecule of a chemical substance, in this case, molecule of drug, that noncovalently binds or adsorbs to a surface of a particulate, in this case, a complexation agent. This type of complexation is termed particulate complexation. Different particulate adsorbents, or complexation agents, have different sorptive properties based on size and shape of particulate, functional groups present at the surface, and the surface irregularity and porosity of the particulate. The utility of particulate complexation has been recognized in other disciplines, including soil sciences, wherein a chemical adsorbent (e.g., alumina, silica gel, activated charcoal) interacts with specific chemicals (frequently contaminants) in soil; the hydrocarbon industry, wherein adsorbents (e.g., polypropylene, vermiculite, perlite, polyethylene, others) are used to clean oil spills or to remove residual oil from drilling and fracking equipment; and industrial coatings (e.g., zeolite, silica gel, aluminum phosphate), wherein adsorbents are used to bind chemical substances for various purposes (i.e., lubrication, surface cooling).

In medical applications, adsorbents are used for the treatment of acute poisoning by ingestion (e.g., activated charcoal, calcium polystyrene sulfate, aluminum silicate) where the adsorbent binds the toxin to limit adsorption from the gut into systemic circulation. In the pharmaceutical industry, principles of adsorption complexation are used to understand chemistry of drug binding to plasma proteins in the blood, drug coatings on solid scaffolds for in situ drug release (e.g., drug-eluting stents), and affixing excipients to insoluble drugs in order to improve oral bioavailability and gut absorption.

The methods and compositions described herein may utilize particulate complexation, wherein complexation agents thus are chemicals compatible with ocular tissues that, when formulated as an irregularly shaped particulates, have the capacity of noncovalently binding drug substance, forming drug substance-complex particulates. One or more drug substance-complex particulates are incorporated and admixed into a hydrophobic dispersal medium to form a stable multiphasic colloidal suspension, that is safely delivered into and around the eye, to produce continuous exposure to predictable therapeutic levels of drug substance in ocular tissues for a desired duration of treatment. Complexation agents are selected from one of six classes of chemical substances, including fatty acid, organic compounds that can form keto-enol tautomer, charged phospholipid, charged protein, nucleic acid, and polysaccharides.

When the drug substance is a prodrug, the conjugation moiety of the prodrug is specifically chosen for its ability to complex, or form noncovalent interactions, with one or more particulate complexation agents to form prodrug-complex particulates. One or more prodrug substance-complex particulates are incorporated and admixed into a hydrophobic dispersal medium to form a stable multiphasic colloidal suspension, that is safely delivered into and around the eye, to produce continuous exposure to predictable therapeutic levels of drug substance in ocular tissues for a desired duration of treatment. Complexation agents are selected from one of six classes of chemical substances, including fatty acid, organic compounds that can form keto-enol tautomer, charged phospholipid, charged protein, nucleic acid, and polysaccharides.

The methods and compositions described herein disclose a new property, not previously recognized, of these six classes of chemical substances, fatty acid, organic compounds that can form keto-enol tautomer, charged phospholipid, charged protein, nucleic acid, and polysaccharides, that, when in the form of an irregularly shaped particulate with irregular surface, can serve as an effective complexation agent for drug substances. The criteria for complexation agent includes the following four features: (1) drug substance binds to the particulate complexation agent and this is demonstrable by microscopy imaging (see FIGS. 4-7); (2) when particulate of substance is added to a solution of drug substance, upon centrifugation and pulldown of the particulates, pharmacologically significant quantities of drug substance are observed to be complexed to the particulates, providing a quantitative metric of binding capacity of the complexation agent (see FIGS. 15, 18, 20, 24); (3) drug substance-complex particulates when resuspended in appropriate dispersal medium, demonstrate partial release of drug, allowing determination of Kd or unbound-bound fraction of drug for a given drug substance-complexation agent pair in a particular dispersal medium (see FIG. 25); and (4) the drug substance-complex particulate provide a useful pharmacokinetic release profile when admixed into the dispersal medium to form the drug substance multiphasic colloidal suspension (see FIG. 9). Collectively, these four properties define a complexation agent and enable the presently described complexation-based XRDDS.

In contrast, spherical particulates with a spherical smooth surface and non-reactive coating, including for example silicone beads, latex beads, and certain polymeric particulates, fail to form stable complexes with drug substance, and therefore may be excluded.

One class of complexation agents is fatty acid, which is a carboxylic acid with an aliphatic chain, which may be either saturated or unsaturated, and may be in the form of a salt or ester (see example in FIG. 4). For example, the fatty acid may have a chemical formula of CH3(CH2)ₙCOOH where n is equal to between 4 and 30. Specific examples of salt form fatty acids include magnesium stearate, magnesium palmitate, calcium stearate, calcium palmitate, and others.

One class of complexation agents is organic compounds that can form keto-enol tautomers. Tautomers refer to molecules capable of undergoing chemical equilibrium between a keto form (a ketone or an aldehyde) and an enol form (an alcohol). Usually, a compound capable of undergoing keto-enol tautomerization contains a carbonyl group (C═O) in equilibrium with an enol tautomer, which contains a pair of doubly bonded carbon atoms adjacent to a hydroxyl (—OH) group, C═C—OH as depicted herein:

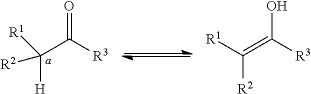

The relative concentration of the keto and enol forms is determined by the chemical properties of the specific molecule and the chemical microenvironment, including equilibrium, temperature or redox state. Organic compounds capable of keto-enol tautomerization include but are not limited to phenols, tocopherols, quinones, ribonucleic acids, and others.

One class of complexation agents is charged phospholipid (see example in FIG. 7). In general, phospholipids consist of a glycerol molecule, two fatty acids, and a phosphate group that is modified by an alcohol, wherein the polar head of the phospholipid is typically negatively charged. Examples include lecithin, phosphatidylcholine, phosphatidylethanolamine, phosphatidylserine, different phospholipids in oil, and many others, which may be used individually or in combination to serve as complexation agents. Anionic phospholipids may comprise one of: phosphatidic acid, phophatidyl serine, sphingomyelin or phophatidyl inositol. In some instances, synthetic, ionizable phospholipids with positive charge can manufactured, including but not limited to examples such as DLin-MC3-DMA. Additional cationic phospholipids may comprise one of: cationic triesters of phosphatidylcholine; 1,2-dimyristoylsn-glycerol-3-phosphocholine (DMPC); 1,2-dioleoyl-sn-glycerol-3-phosphocholine (DOPC); 1,2-bis(oleoyloxy)-3-(trimethylammonio)propane (DOTAP); 1,2-dioleoyl-sn-glycerol-3-phosphoethanolamine (DOPE); 1,2-dipalmitoyl-sn-glycerol-3-phosphocholine (DPPC); 1,2-dioleoyl-sn-glycerol-3-ethylphosphocholine (EDOPC); 1,2-dimyristoyl-sn-glycerol-3-ethylphosphocholine (EDMPC); 1,2-dipalmitoyl-sn-glycerol-3-ethylphosphocholine (EDPPC). In pharmaceutical sciences, phospholipids have been used for drug formulation and delivery applications to improve bio-availability, reduced toxicity, and improved cellular permeability. However, in the methods and compositions described herein, phospholipids may be used as a complexation agent particulate to noncovalently bind the drug substance and form drug substance-complex particulates for the purpose of regulating free drug substance in the dispersal medium of the stable multiphasic colloidal suspension in which the drug substance-complex particulates are incorporated and dispersed therein.

One class of complexation agents is charged protein. Proteins are large biomolecules and macromolecules that comprise one or more long changes of amino acid residues. Amino acids that make up proteins may be positive, negative, neutral, or polar in nature, and collectively, the amino acids that comprise the protein give it its overall charge. A variety of proteins, based on size, molecular weight, ability to readily form particulates, and compatibility with ocular tissues could serve as complexation agents (see example in FIG. 5). The charge of the protein will determine its compatibility with a specific drug substance such that negatively charged proteins will readily complex with positively charged drug substance, while positively charged proteins (e.g., Arg-Gln-Ile-Arg-Arg-Ile-Ile-Gln-Arg-NH₂ and synthetic peptides with positive charge) will readily complex with negatively charged drug substance. Examples of proteins that could serve as complexation agents include albumin and collagen.

One class of complexation agents is nucleic acids, biopolymer macromolecules composed of nucleotides, comprised of a 5-carbon sugar, a phosphate group, and a nitrogenous base. The importance of nucleic acids for biologic function and encoding genetic information is well established. However, nucleic acids also have a variety of applications, including nucleic acid enzymes (e.g., carbon nanomaterials), aptamers (e.g., for formation of nucleic acid nanostructures and therapeutic molecules that function in an antibody-like fashion), and aptazymes (e.g., which can be used for in vivo imaging). In pharmaceutical sciences, specially engineered nucleic acids have been considered and applied for use in carrier-based systems in which the nucleic acid serves as a carrier system for various types of drugs. However, in the methods and compositions described herein, nucleic acids may not be considered a carrier system but rather as a complexation agent, as they are highly negatively charged and thus, formulated as a particulate, could then serve as a complexation agent for positively charged drug substance.

One class of complexation agent is polysaccharides, long chain polymeric carbohydrates composed of monosaccharide units bound together by glycosidic linkages. Frequently, these are quite heterogenous, containing slight modifications of the repeating monosaccharide unit. Depending on structure, they can be insoluble in water. Complexation of polysaccharide particulate complexation agents to drug substances can occur through various electrostatic interactions and is influenced by charge density of drug substance and polysaccharide, ratio of polysaccharide complexation agent drug substance, ionic strength, and other properties (see example in FIG. 6). Examples of polysaccharides that could serve as complexation agents include a ringed polysaccharide molecule, cyclodextrins, a clathrate, cellulose, pectins, or acidic polysaccharides, polysaccharides that contain carboxyl groups, phosphate groups, or other similarly charged groups.

In the methods and compositions described herein, a selected drug substance has specific avidity for, and complexes with, a given complexation agent, forming a drug substance-complex particulate. This avidity can be measured as Kd, the unbound-bound fraction of a drug substance for a given drug substance-complex particulate in a selected dispersal medium.

Another property of drug substance-complex particulate is the binding capacity, defined as a quantity of drug substance bound to a known quantity of complexation agent.

The avidity and binding capacity of the drug substance for a particular complexation agent thus serves to limit the free drug available for release from the drug substance-complex particulate for a given dispersal medium.

Drug substance formulated in the present extended release drug delivery system (XRDDS), the multiphasic colloidal suspension, may include various small polypeptides, proteins, aptamers, other nucleic acid drugs, hydrophobic chemicals, hydrophilic chemicals, and other chemical compounds used for therapeutic purposes that are capable of directly forming noncovalent complexes to one of six classes of complexation agents: fatty acid, organic compounds that can form keto-enol tautomers, charged phospholipid, charged protein, ribonucleic acid, and polysaccharide.

The drug substance directly forms noncovalent avid interactions (or binding) to one of six different classes of substances formulated as irregularly shaped particulates: fatty acid, organic molecules that can form keto-enol tautomers, charged phospholipid, charged protein, nucleic acid, and polysaccharides. The resultant drug substance-complex particulates admixed into dispersal medium regulates the release of free, unbound drug within the multiphasic colloidal suspension, enabling controlled, extended release from the formulated implant upon administration into ocular physiologic environment (see FIGS. 1-3).

The drug substance formulated in the multiphasic colloidal suspension may also be a prodrug of any active pharmaceutical ingredient (API) linked via cleavable covalent bond to a conjugation moiety (see FIG. 1), wherein the conjugation moiety forms complexes with one of six classes of complexation agents: fatty acid, organic compounds that can form keto-enol tautomers, charged phospholipid, charged protein, ribonucleic acid, and polysaccharide.

The prodrug has formula (I):

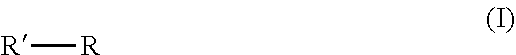

where R′ is any active pharmaceutical ingredient (API) that is covalently linked via cleavable bond to R, a conjugation moiety that forms noncovalent complexes with one of five classes of complexation agents, and the covalent bond linking R′ and R may be removed by enzymatic cleavage, catalysis, hydrolysis, or other reaction to yield free API R′ and conjugation moiety R, where R is selected from: a C4-C30 lipid moiety (fatty acid or fatty alcohol), an C4-C30 straight-chain or branched aliphatic moiety, a 2-mer to 30-mer peptide moiety, a pegylated moiety, or a carbohydrate moiety.

The covalently linked conjugation moieties of drug substances form noncovalent avid interactions, binding to one of six different classes of substances formulated as irregularly shaped particulates: fatty acid, organic molecules that can form keto-enol tautomers, charged phospholipid, charged protein, nucleic acid, and polysaccharides (see FIGS. 4C, 5C, 6C, 7C). The formation of prodrug-complex particulates optimizes the API's physicochemical properties for compatibility with the multiphasic colloidal suspension, wherein the prodrug-complex particulates admixed into dispersal medium regulates the release of free, unbound prodrug within the multiphasic colloidal suspension, enabling controlled, extended release from the formulated implant upon administration into ocular physiologic environment.

When the drug substance is a prodrug, a key feature of the prodrug is that the bond linking API to the conjugation moiety is readily cleaved by enzymatic reaction, catalysis, hydrolysis, or other chemical reaction. Upon cleavage of this bond in the prodrug, the released API retains full bioactivity for its mechanism of action (see example in FIGS. 29A-29C).

The cleavable covalent bond may comprise one of: an ester bond, a hydrazone bond, an imine bond, a disulfide bond, a thioester bond, a thioether bond, a phosphate ester bond, a phosphonate ester bond, a boronate ester bond, an amide bond, a carbamate ester bond, a carboxylate ester bond, and a carbonate ester bond.

In general, the conjugation moiety, R, to which the API is covalently linked, is not selected on the basis of bioactivity for a target or mechanism of action.

Although not a preferred embodiment, disclosed herein are drug substances comprised of homo- or hetero-dimers, trimers, multimers of any drug substance, either linked together directly or indirectly to a chemical substance that serves a linker moiety, which could functionally serve as a cleavable conjugation moiety.

As described herein, the API, R′, may be covalently linked to conjugation moiety R, selected from among one of the following five classes of chemical substances: a C4-C30 lipid moiety, a C4-C30 straight-chain or branched aliphatic moiety, a 2-mer to 30-mer peptide moiety, a pegylated moiety, or a carbohydrate moiety.

One class of conjugation moieties is C4-C30 lipid moiety, with or without a preceding linker moiety that bonds the lipid moiety to the API (see example in FIG. 27A). Herein, lipid is defined as organic compounds that are insoluble in water but soluble in organic solvents. Lipids include fatty acids, fatty alcohols, glycerolipids, glycerophospholipids, sphingolipids, saccharolipids, polyketides (derived from condensation of ketoacyl subunits), sterol lipids, prenol lipids (derived from condensation of isoprene subunits), phospholipids, oils, waxes, and steroids.

One class of conjugation moieties is C4-C30 straight-chain or branched aliphatic moiety, with or without a preceding linker moiety that bonds the aliphatic hydrocarbon, to the API. This class include alkanes, alkenes, and alkynes and other hydrocarbon moieties made up of 4 to about 30 carbons.

One class of conjugation moieties is peptide moiety, with or without a preceding linker moiety that bonds the peptide to API (see examples in FIGS. 27B-27C), wherein the peptide moiety comprises a natural or synthetic amino acid polymer or polypeptide chain with length of 2-mer to 30 mer, which may be anionic, cationic, or neutral in charge and contain homogeneous or heterogeneous amino acid repeats.

Anionic peptide moiety may include at least one of: poly-glutamate, poly-aspartate or a combination of glutamate and aspartate.

Cationic peptide moiety may include at least one of: poly-arginine, poly-lysine, poly-histidine, a combination of arginine and lysine, a combination of arginine and histidine, a combination of histidine and lysine, a combination of arginine, histidine, and lysine.

The peptide moiety may have one or more PEGylation sites for addition of polyethylene glycol (PEG) groups or may have one or more sites for modification by addition of sugar or carbohydrate molecules, including glycosylation.

One class of conjugation moieties is pegylated compound moiety, with or without a preceding linker moiety that bonds the pegylated compound to the API (see example in FIG. 27D), including polyethylene glycol (PEG) polymers of linear, branched, Y-shaped, or multi-arm geometries, pegylated peptides or proteins, or pegylated succinates such as succinimidyl succinate.

One class of conjugation moieties is carbohydrate molecular moiety, with or without a preceding linker moiety that bonds the carbohydrate to the API, including but not limited to monosaccharides or oligosaccharides of 2 to 20 sugars. The carbohydrate molecule may comprise one or more of: glucose, galactose, lactose, mannose, ribose, fucose, N-acetylgalactosamine, N-acetylglucosamine, N-acetyleneuraminic acid, or an epimer or derivative of any of these.

One example of how a prodrug may be incorporated into a multiphasic colloidal suspension is from among the class of mitochondria-targeted tetrapeptides (MTT), which can be used to form a prodrug that is a product of a condensation or esterification reaction, of formula, (II):

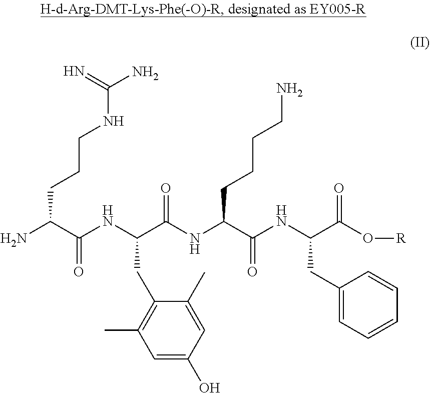

where R is covalently linked via ester bond at the hydroxyl group of the amino acid in the 4th position of the MTT and is selected from among one of the following five classes of chemical substances: a C4-C30 lipid moiety, an C4-C30 straight-chain or branched aliphatic moiety, a 2-mer to 30-mer peptide moiety, a pegylated moiety, or a carbohydrate moiety (FIG. 26).

One specific example of EY005-prodrug includes EY005-stearyl (depicted in FIG. 27A), wherein EY005 is linked via ester bond to stearyl alcohol, one member from the group of long-chain saturated fatty alcohols. On cleavage of the ester bond, the prodrug EY005-stearyl releases the EY005 MTT. To demonstrate this experimentally (FIG. 28), EY005-stearyl was incubated at 37° C. in vitro with carboxyesterase (0.1 μg/mL), to simulate the ocular physiologic environment and the type of esterase that is readily abundant therein, within the vitreous. Incubation of EY005-stearyl with carboxyesterase produced rapid cleavage of the prodrug ester bond, releasing EY005, as evident by high performance liquid chromatography (HPLC) analysis and quantification of EY005 MTT and EY005-stearyl prodrug in solution (FIG. 28B). Upon addition of EY005-stearyl prodrug to phosphate-buffered saline solution at 37° C. without esterase, the ester bond of the EY005-stearyl prodrug cleaves more slowly (˜36 hours) by hydrolysis (FIG. 28C). Thus, in ocular physiologic system, the covalent bond of the prodrug linking MTT to inactive conjugation is readily cleaved either by enzymatic cleavage or more slowly by hydrolysis, releasing the active MTT.

Further, upon cleavage of the covalent bond of the drug substance, the API, the native MTT peptide, retains bioactivity for treatment of mitochondrial dysfunction. For example, as depicted in FIGS. 29A-29C, in an in vitro cell culture model of dry AMD, EY005-stearyl (5 μM) was added to RPE cells (which possess endogenous esterases) with mitochondrial dysfunction induced by exposure to hydroquinone (HQ). EY005-stearyl effectively reversed HQ-induced mitochondrial dysfunction in RPE cells (as depicted by cellular flavoprotein-autofluorescence), with efficacy equivalent to treatment with EY005 native peptide (5 μM). EY005-stearyl was also preincubated with carboxyesterase (0.1 μg/mL) in separate media. Recovered media containing cleaved EY005 (5 μM) was added to this RPE cellular model of mitochondrial dysfunction, and this was similarly effective and equipotent to EY005 native peptide for the reversal of RPE mitochondrial dysfunction. Thus, these studies affirm that the active MTT that is cleaved from the drug substance retains essential and unmodified bioactivity for the treatment of mitochondrial dysfunction.

In some instances, a conjugation moiety, which may be combine elements from two or more of these classes, may serve as as a multimeric linker moiety that is covalently linked to multiple molecules of the API to form dimers and/or multimers. Such linkers capable of generating dimers or multimers of mitochondria targeting peptides may be referred to as “multimerization domains.”

Prodrug with multimerization domain has formula (III):

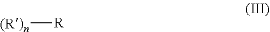

wherein R is a linker or multimerization domain which is covalently linked to multiple API R′, to form dimers or multimers of the API and n is equal to 2 to about 100. Examples include PEG polymers (FIG. 27D), polyvinyl alcohol (PVA) polymers, or polypeptides, where the linker conjugation moiety R is covalently linked to two or more molecules of the API R′, to form dimers, trimers, multimers, etc. In some cases, the multimerization domains have alcohols, i.e., multiple “—OH” groups, to which the API units R′ are bound. In this setting, multiple API covalently linked (e.g., via ester or another dynamic covalent bond) to the multimerization domain may be referred to an API multimer.

One example of such a prodrug multimer is the mitochondrial-targeted tetrapeptide H-d-Arg-DMT-Lys-Phe linked to PVA compound, with the formula, where “n” is number comprising PVA polymer:

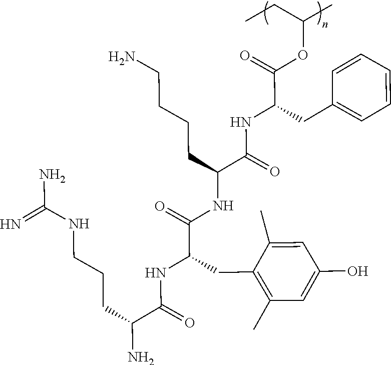

The dispersal medium of the drug substance multiphasic colloidal suspension is defined herein as a hydrophobic liquid into which drug substance and particulate complexation agents are admixed to form a stable multiphasic colloidal suspension.

The criteria that define a stable multiphasic colloidal suspension include uniform mixture and distribution of the drug substance-complex particulates without settling, separation, or dissociation of the particulates for the prespecified duration of the implant's lifetime, after exposure to an ocular physiologic environment in vitro (i.e., 37° C., buffered saline, vitreous enzymes, dilute serum) or in vivo when injected into the eye. The stability is also dependent on the relative percentage of drug substance-complex particulates to oil (weight to weight) and the size and mass of the particulates.

The methods and compositions described herein describe previously unrecognized properties of certain oils that allow them to serve as effective dispersal medium (see example in FIGS. 12A-12F). These include hydrophobicity, high starting viscosity, and other properties that allow it to form a stable multiphasic colloidal suspension when admixed with drug substance-complex particulates.

Four classes of oils that meet these criteria for dispersal medium include saturated fatty acid methyl esters, unsaturated fatty acid methyl esters, saturated fatty acid ethyl esters, or unsaturated fatty acid ethyl esters. A dispersal medium can be an individual oil from one of these classes or can be designed as a mixture of oils with different viscosity values that are specifically designed and admixed to achieve the desired goal of a stable colloidal suspension.

Saturated fatty acid methyl esters that may serve as dispersal medium include: methyl acetate, methyl propionate, methyl butyrate, methyl pentanoate, methyl hexanoate, methyl heptanoate, methyl octanoate, methyl nonanoate, methyl decanoate, methyl undecanoate, methyl dodecanoate (methyl laurate), methyl tridecanoate, methyl tetradecanoate, methyl 9(Z)-tetradecenoate, methyl pentadecanoate, methyl hexadecanoate, methyl heptadecanoate, methyl octadecenoate, methyl nonadecanoate, methyl eicosanoate, methyl heneicosanoate, methyl docosanoate, methyl tricosanoate, and others.

Unsaturated fatty acid methyl esters that may serve as dispersal medium include: methyl 10-undecenoate, methyl 11-dodecenoate, methyl 12-tridecenoate, methyl 9(E)-tetradecenoate, methyl 10(Z)-pentadecenoate, methyl 10(E)-pentadecenoate, methyl 14-pentadecenoate, methyl 9(Z)-hexadecenoate, methyl 9(E)-hexadecenoate, methyl 6(Z)-hexadecenoate, methyl 7(Z))-hexadecenoate, methyl 11(Z)-hexadecenoate.

Saturated fatty acid ethyl esters that may serve as dispersal medium include: ethyl acetate, ethyl propionate, ethyl butyrate, ethyl pentanoate, ethyl hexanoate, ethyl heptanoate, ethyl octanoate, ethyl nonanoate, ethyl decanoate, ethyl undecanoate, ethyl dodecanoate (ethyl laurate), ethyl tridecanoate, ethyl tetradecanoate, ethyl 9(Z)-tetradecenoate, ethyl pentadecanoate, ethyl hexadecanoate, ethyl heptadecanoate, ethyl octadecenoate, ethyl nonadecanoate, ethyl eicosanoate, ethyl heneicosanoate, ethyl docosanoate, ethyl tricosanoate.

Unsaturated fatty acid ethyl esters that may serve as dispersal medium include: ethyl 10-undecenoate, ethyl 11-dodecenoate, ethyl 12-tridecenoate, ethyl 9(E)-tetradecenoate, ethyl 10(Z)-pentadecenoate, ethyl 10(E)-pentadecenoate, ethyl 14-pentadecenoate, ethyl 9(Z)-hexadecenoate, ethyl 9(E)-hexadecenoate, ethyl 6(Z)-hexadecenoate, ethyl 7(Z))-hexadecenoate, ethyl 11(Z)-hexadecenoate.

In contrast, certain other oils and viscous substances including water, silicone oil, viscous gelatin, and viscous proteoglycan fail to form a stable multiphasic colloidal suspension or rapidly decompensate when exposed to a physiologic ocular microenvironment (e.g., 37° C., buffered saline, vitreous enzymes, dilute serum) or in vivo when injected into the eye (see examples in FIGS. 10, 11, 13, 14).

Complexation of drug substance to particulate complexation agents within the dispersal medium serves to limit the release of free drug substance into the dispersal medium. While the dispersal medium restricts access of water to the drug substance-complex particulates, free, unbound drug substance diffuses freely within the dispersal medium, and the dispersal medium does not retain the free, unbound drug, which can diffuse out of the multiphasic colloidal suspension.

The drug substance multiphasic colloidal suspension can be designed by specific process to meet a prespecified release rate and amount of drug substance, by varying the ratios and amounts of different drug substance-complex particulates, with different Kd and binding capacity (see FIGS. 2 and 16). The property of Kd is a measure of avidity of a drug substance for a given complexation agent and is defined as the unbound-bound fraction of drug substance for a drug substance-complex particulate in a given dispersal medium. Specific Kd value can be measured by specified release assay, as described herein. The property of binding capacity is defined as a maximal amount of drug that is bound to a known quantity of complexation agent.

Release of drug substance from the implant is determined in part by the unbound fraction within the dispersal medium, which is in turn determined in part by the Kd values and the binding capacity values for different drug substance-complex particulates. Knowledge of the Kd and binding capacity (see FIGS. 15, 18, 20, 24) allows the choice of specific combinations of different prodrug-complexation agent particulates to regulate the unbound fraction of drug within the dispersal medium over time and to thus achieve a prespecified release kinetics profile (see FIGS. 2, 3, and 16).

For example, the addition of drug substance-complex particulate with high binding capacity and high Kd, indicating low avidity of drug substance to the complex particulate, can be used to create a short-term increased rate of release, or initial burst. The addition of drug substance-complex particulates with high binding capacity and moderate Kd, indicating moderate avidity of drug substance to the complex particulate, can be used to create a long-term lower rate of release, to extend the duration of drug substance release from the implant. The combination of these two types of drug-substance particulates can be selected and admixed, in desired ratio and concentration, to achieve to create an implant with two-phase release kinetics of drug substance from the implants (FIGS. 2 and 3). An implant with this release kinetic profile would be useful for diseases that require a “loading” phase to treat and reverse established disease pathobiology, while the second “steady-state” phase would be effective for preventing onset of new or recurrent disease.

In another example, the addition of drug substance-complex particulate with high binding capacity and high Kd, indicating low avidity of drug substance to the complex particulate, can be used to create a short-term increased rate of release, or initial burst. The addition of drug substance-complex particulates with high binding capacity and moderate Kd, indicating moderate avidity of drug substance to the complex particulate, can be used to create a long-term lower rate of release, to extend the duration of drug substance release from the implant. The addition of drug substance-complex particulates with high binding capacity and low Kd, indicating high avidity of drug substance to the complex particulate, would release late an to create a late-term burst in the implant's lifetime. The combination of these three types of drug-substance particulates can be selected and admixed, in desired ratio and concentration, to achieve to create an implant with three-phase release kinetics of drug substance from the implants (FIG. 3). An implant with this release kinetic profile would be useful for diseases that require a “loading” phase to treat and reverse established disease pathobiology, while the second “steady-state” phase would be effective for preventing onset of new or recurrent disease, and yet a third phase of “late burst” would be useful for diseases in which there is diminished response of the target to the drug late in the life of the implant due to tachyphylaxis or other mechanisms that mediate downregulation of the drug target or diminished responsiveness to the drug substance.

In such examples, the combined effect for a combination of two or more drug substance-complex particulates incorporated into selected dispersal medium is release of the drug substance in two or more phases based on the integral of release rates from the individual drug-complexation agent particulate components that are incorporated and dispersed into the drug substance multiphasic colloidal suspension (see FIG. 2).

The actual release kinetics of achieved by the drug substance multiphasic colloidal suspension in in vivo vitreous concentrations may meet or exceed EC₅₀ for an extended-release duration of 1 month or more. The EC₅₀ reflects the concentration of the drug substance that achieves 50% of the maximal response therapeutic effect, for the given mechanism of action of the drug substance.

In formulations of drug substance multiphasic colloidal suspension with two-phase release kinetics, the concentration of drug substance in the vitreous may exceed the reversal EC₅₀ (i.e., drug concentration required to achieve 50% of the maximal effect) during the initial burst phase and subsequently exceed the prevention EC₅₀ for the second (steady-state) phase, wherein prespecified release kinetics and desired duration of drug release were achieved by specific design and use of different drug substance-complex particulates in the multiphasic colloidal suspension as described herein.

Formulations of the drug substance multiphasic colloidal suspension can be delivered as one of three different implant modalities, including a flowable bolus implant, an erodible or non-bioerodible tube implant filled with drug substance multiphasic colloidal suspension, or a solid mold of drug substance multiphasic colloidal suspension fashioned into specific size and shape, dried and hardened and configured for implantation (FIG. 33). Any of these formulations may be injected in and around the eye, i.e., into the vitreous humor, into the aqueous humor, into the suprachoroidal space, under the retina, under the conjunctiva, beneath Tenon's capsule, or into orbital tissue, to produce sustained release of therapeutic levels of drug substance within ocular tissues for desired duration (1 to 12 months), for the treatment of various diseases and disorders.

The multiphasic colloidal suspension described herein can incorporate a variety of drug substances that directly form noncovalent complex with particulate complexation agents, as well as a variety of prodrugs, comprised of an active pharmaceutical ingredient (API) linked via cleavable covalent bond to a conjugation moiety, wherein the conjugation moiety of the prodrug forms noncovalent complex with particulate complexation agents (FIGS. 1, 2, 3). Specifically, the multiphasic colloidal suspension can incorporate various hydrophobic chemicals, hydrophilic chemicals, small polypeptides, proteins, aptamers, other nucleic acid drugs, and other chemical compounds.

Several examples are discussed herein, to demonstrate principles of complexation for drug delivery.

For example, a fluoresceinated, cationic small molecule was admixed with known quantities of selected individual complexation agents (see FIGS. 4F, 5F, 6F, 7F). Different fluoresceinated small molecule-complex particulates were then admixed to an appropriate dispersal medium and visualized under fluorescent microscopy. Using this approach, fluoresceinated small molecule was observed to form fluoresceinated small molecule-complex particulates with several different complexation agents, but did not adsorb to silica microbeads (FIG. 8F, not a complexation agent).

In another example, a fluoresceinated, C12 (i.e., 12-carbon) lipid molecule was admixed with known quantities of selected individual complexation agents (see FIGS. 4E, 5E, 6E, 7E). Different fluoresceinated lipid molecule-complex particulates were then admixed to an appropriate dispersal medium and visualized under fluorescent microscopy. Using this approach, fluoresceinated lipid molecule was observed to form fluoresceinated lipid molecule-complex particulates with several different complexation agents but did not adsorb to silica microbeads (FIG. 8E, not a complexation agent).

In another example, the tetrapeptide H-d-Arg-DMT-Lys-Phe was fluorescently labeled with fluorescein isothiocyanate (FITC) was admixed with known quantities of selected individual complexation agents (see FIGS. 4B, 5B, 6B, 7B). Different fluoresceinated small molecule-complex particulates were then admixed to an appropriate dispersal medium and visualized under direct fluorescent microscopy. Using this approach, when admixed with different complexation agents (e.g., magnesium stearate, albumin, cyclodextrin, lecithin), FITC-labeled H-d-Arg-DMT-Lys-Phe did not produce visible drug-complex particulates. FITC-labeled H-d-Arg-DMT-Lys-Phe adsorbed to silica microbeads (not a complexation agent, FIG. 8B). This did not represent stable complexation as the weakly avid interactions caused fluorescence of the particulates to disappear once colloidal suspension of H-d-Arg-DMT-Lys-Phe/microbeads was added to an ocular physiologic environment in vitro (see FIG. 25A).

In another example, the same tetrapeptide H-d-Arg-DMT-Lys-Phe was linked by ester bond to stearyl alcohol, to form the prodrug H-d-Arg-DMT-Lys-Phe(O)-stearyl. The prodrug H-d-Arg-DMT-Lys-Phe(O)-stearyl 1 was fluorescently labeled with FITC and admixed with different complexation agents (see FIGS. 4C, 5C, 6C, 7C). The resultant mixture was then visualized under direct fluorescence microscopy. Using this approach, FITC-labeled H-d-Arg-DMT-Lys-Phe(O)-stearyl (in which the tetrapeptide was labeled with FITC) was observed to form drug-complex particulates with several different complexation agents: magnesium stearate (as previously described, and as expected); albumin, a large, charged carrier protein; and cyclodextran, a large cyclic carbohydrate molecule, and lecithin, a charged phospholipid. In contrast, FITC-labeled H-d-Arg-DMT-Lys-Phe(O)-stearyl did not adsorb to silica microbeads (FIG. 8C, not a complexation agent).

Since only H-d-Arg-DMT-Lys-Phe(O)-stearyl prodrug with conjugation moiety formed drug-complex particulates, it is inferred that complex formation was mediated by the conjugation moiety of the prodrug. To assess this, FITC-labeled H-d-Arg-DMT-Lys-Phe(O)-stearyl (in which the tetrapeptide was labeled with FITC) that had been admixed with complexation agent was treated with an aqueous solution of carboxyesterase (0.1 μg/mL) to hydrolyze the ester bond of the prodrug, releasing the fluorescent peptide. Complexed particulates were no longer fluorescently labeled by microscopy (see FIGS. 4D, 5D, 6D, 7D), affirming that complexation of the prodrug is specifically mediated by its conjugation moiety and validating the concept of using a prodrug with compatible conjugation moiety to mediate complex formation.

Further, as described herein, formation of drug-complex particulates in which the complexation agent has high avidity for the drug can be quantified and verified experimentally. For example, the prodrug H-d-Arg-DMT-Lys-Phe(O)-stearyl was admixed with known quantities of selected individual complexation agents (see FIGS. 24 and 25). The H-d-Arg-DMT-Lys-Phe(O)-stearyl-complexation agent mixture was then added to an appropriate dispersal medium (in this case, methyl laurate), and centrifuged to “pull down” or separate H-d-Arg-DMT-Lys-Phe(O)-stearyl bound to complexation agent from unbound prodrug present in the dispersal medium. HPLC analysis of pulled down particulates and dispersal medium from H-d-Arg-DMT-Lys-Phe(O)-stearyl content determined the fraction of prodrug that is bound to the complexation agent and calculation of the Kd value, the unbound to bound coefficient, and binding capacity for the prodrug-complexation agent particulate (FIG. 24). Using this type of assay, Kd values and binding capacity can be generated for specific prodrug-complexation agent pairs in a selected dispersal medium.

Further, the addition of conjugation moiety to the H-d-Arg-DMT-Lys-Phe native peptide (designated as EY005) enabled noncovalent complexation to various complexation agents in a manner that dramatically altered the kinetic release profile from multiphasic colloidal suspension added to an in vitro ocular physiologic environment (FIG. 25). In various formulations of EY005 admixed with individual complexation agents in dispersal medium, the EY005 native peptide rapidly “dumped” from all formulations that 100% of the EY005 peptide drug was fully released from all formulations by day 4 (FIG. 25A). In contrast, various formulations of H-d-Arg-DMT-Lys-Phe(O)-stearyl (designated as EY005-stearyl) complexed to individual complexation agents in dispersal medium (i.e., the multicolloidal suspension) produced sustained release of EY005-stearyl prodrug over time, with variable release kinetics based on the Kd and binding capacity of individual complexation agents, when added to an in vitro ocular physiologic environment (FIG. 25B).

In some embodiments, formulations of the drug substance (in this case EY005-prodrug) in multiphasic colloidal suspension comprised of two distinct prodrug-complex particulates, in this example magnesium stearate and albumin, produce a unique kinetic release profile that reflects an integral of the ratio and concentration of the distinct prodrug-complex particulates that have their own unique Kd and binding capacity properties (FIG. 25B).

Thus, in some examples, the formation of a prodrug substantially alters the physicochemical properties of the API to enable complexation and optimize its compatibility for formulation in the multiphasic colloidal suspension. In the example of FIG. 25, API H-d-Arg-DMT-Lys-Phe is highly hydrophilic, and as noted above, did not produce visible drug-complex particulates on admixture with complexation agents. Kd and binding capacity of the unmodified API H-d-Arg-DMT-Lys-Phe are substantially different. Linkage via ester bond to stearyl alcohol produced the prodrug H-d-Arg-DMT-Lys-Phe(O)-stearyl, is highly hydrophobic as compared to the unmodified API (FIGS. 24 and 25). Further, the high avidity interaction between the hydrophobic, long-chain fatty alcohol of the conjugation moiety of this MTT-prodrug and particulate complexation agents serves to bind the MTT-prodrug and limits the free, unbound MTT-prodrug that is available for release from the dispersal medium in which the MTT-prodrug-complex particulates are dispersed (FIG. 25).

Another specific example of prodrugs includes H-d-Arg-DMT-Lys-Phe(O)-tri-arginine (triArg) (FIG. 27C), wherein H-d-Arg-DMT-Lys-Phe is linked via ester bond to arginine trimer/tripeptide, a positively charged peptide conjugation moiety that readily forms noncovalent complex with negatively charged particulate complexation agents to form MTT-prodrug-complex particulates. The high avidity interaction between the positively conjugation moiety of this MTT-prodrug and the negative charge of the particulate complexation agent serves to bind this MTT-triArg prodrug and limits the free, unbound MTT-prodrug that is available for release from the dispersal medium in which the MTT-prodrug-complex particulate is dispersed.

Another specific example of prodrugs includes H-d-Arg-DMT-Lys-Phe(O)-tri-glutamate (triGlu) (FIG. 27B), wherein H-d-Arg-DMT-Lys-Phe is linked via ester bond to glutamate trimer/tripeptide, a negatively charged peptide conjugation moiety that readily forms noncovalent complex with positively charged particulate complexation agents to form MTT-prodrug-complex particulates. The high avidity interaction between the negatively charged conjugation moiety of this MTT-prodrug and the positive charge of the particulate complexation agent serves to bind MTT-triGlu prodrug and limits the free, unbound MTT-prodrug that is available for release from the dispersal medium in which the MTT-prodrug-complex particulate is dispersed.

Several examples are discussed herein, to specifically identify and differentiate substances that can (and substances that cannot) serve as dispersal medium.

Herein, a dispersal medium is defined as a hydrophobic, viscous oil that when admixed with drug substance-complex particulates, can form a stable multiphasic colloidal suspension, which is formed into an implant for administration in or around the eye. Herein, colloidal indicates that the particulates are uniformly dispersed and stable indicates that the particulates remain dispersed without settling or migration for the duration of the implant's intended lifetime.

To better understand these properties and identify liquid substances that could serve as dispersal medium, fluorescent particulate beads of two different sizes, 3 μm (micrometer, or micron) and 10 μm, were used as surrogates for drug substance-complex particulates (to facilitate visualization and imaging of particulates). These fluorescent particulate beads were suspended in various liquids in small shallow cylindrical wells, which were then assessed by confocal fluorescent microscopy to assess the distribution of the particulate beads and to ascertain, via the confocal functionality that assesses various depths of the liquid, whether any settling of fluorescent bead particulates occurred.

For example, when fluorescent particulate beads were admixed in water (FIGS. 10A-10F) and in silicone oil (FIGS. 11A-11F), they demonstrated substantially higher number and density of particulate beads in the bottom levels of the fluid, and relatively much fewer beads in the upper portion of the liquid. Thus, water and silicone oil did not uniformly disperse particulates, and no colloidal suspension was formed, since the particulates settled within the liquid medium.

In another example, fluorescent particulate beads were admixed in the fatty acid methyl ester methyl laurate (FIGS. 12A-12F). Confocal microscopy demonstrated uniform distribution of the particulates regardless of depth of the liquid, indicating that methyl laurate uniformly dispersed particulates, forming a multiphasic colloidal suspension. Examination of this suspension in contact with an ocular physiologic environment (which contains enzymes and proteins typically contained in ocular tissues) over time, at 1 day, 1 week, and 1 month, demonstrated the stability of uniform distribution of particulates without migration within the colloidal suspension.

In another example, fluorescent particulate beads were admixed in 2% gelatin (FIG. 13A-13F). Confocal microscopy demonstrated uniform distribution of the particulates regardless of depth of the liquid, indicating that 2% gelatin uniformly dispersed particulates, forming a colloidal suspension. However, following placement of the 2% gelatin colloidal suspension into an in vitro ocular physiologic environment (which contains collagenase), the distribution of particulates within the suspension did not remain stable over time; the particulates migrated and settled, indicating that a gelatin-based medium could not maintain the stability of the colloidal suspension over time.

Several examples are discussed herein, to demonstrate proof-of-concept for formulation and sustained release of various drug substances in the multiphasic colloidal suspension.

For example, formulations of the hydrophobic small molecule fluocinolone acetonide (FA) in the multiphasic colloidal suspension were developed (FIGS. 15-17). FA was admixed with different particulate complexation agents, to form various FA-complex particulate formulations. Properties of Kd and binding capacity were calculated for each FA-complex particulate, in dispersal medium (FIG. 15). A two-phase kinetic release profile was desired in this example. Based on this, complexation agents of magnesium stearate and lecithin were selected for incorporation, along with FA, into dispersal medium methyl laurate, in specific ratio and concentration to achieve a two-phase release for a bio-erodible tube formulation of FA multiphasic colloidal suspension. Formulations were iteratively refined by adjusting the ratio of FA-complexation agent particulates for a given payload of drug, to achieve approximately six-month duration of release, with an initial burst phase release followed by steady-state release. FIG. 16B illustrates other formulations, wherein varying the ratio of drug-complex particulates altered the kinetics of release, as the proportion of complexation agent (KET) with higher avidity (Kd) was increased across the formulations, for a given payload of FA. For various formulations of FA in multiphasic colloidal suspension (FIG. 17), there was strong correlation between kinetics of drug release in vitro (curves) and kinetics of release in vivo within the eye (colored dots at specific time points reflecting retina tissue levels).

In another example, formulations of the hydrophilic small molecule dexamethasone phosphate (DexPh) in the multiphasic colloidal suspension were developed (FIGS. 18-19). Properties of Kd and binding capacity were calculated for each DexPh-complex particulate, in dispersal medium (FIG. 18). To understand how physicochemical properties of the drug substance influence interactions with complexation properties, DexPh and FA (at same payloads) was each admixed with same particulate complexation agents (magnesium stearate and tocopherol) and dispersal medium (methyl laurate) (FIG. 19, curve for DexPh, curve for FA). Formulation of DexPh demonstrated a rapid and excessive release, or “dump” of DexPh. The addition of a different complexation agent, lecithin, with reduction in ratios of other complexation agents, for a given payload (orange curve) altered the kinetic release profile to minimize dump of DexPh and provide more desirable sustained release profile, demonstrating the importance of selecting complexation agents on the basis of their favorable noncovalent complex formation with specific drug substance of interest.

In another example, formulations of the hydrophilic small molecule sunitinib malate in the multiphasic colloidal suspension were developed (FIGS. 20-22). Sunitinib was admixed with different particulate complexation agents, to form various sunitinib-complex particulate formulations (FIG. 20). Complexation of sunitinib to selected complexation agents was visually confirmed by admixture and pulldown of sunitinib-complex particulates, which was confirmed since by the yellow-orange color of particulates (sunitinib has orange coloration) (FIG. 21). Formulations of sunitinib in a bio-erodible tube formulation of sunitinib multiphasic colloidal suspension were designed and manufactured and produced desired tissue levels and durability of release when implanted in vivo in rabbit eyes (FIG. 22).

For example, formulations of the hydrophobic small molecule axitinib in the multiphasic colloidal suspension were developed. A formulation with single-phase kinetic release profile was desired. Based on this, axitinib was admixed with complexation agent with high binding capacity and low Kd (indicating high affinity) in selected dispersal medium formulated as a bolus implant that produced a slow release formulation, with detectable drug in tissue and desired durability of release (FIG. 23).

In another example, formulations of prodrug H-d-Arg-DMT-Lys-Phe(O)-stearyl, designated as EY005-stearyl, in the multiphasic colloidal suspension were developed (FIGS. 24-32). EY005-stearyl was admixed with different particulate complexation agents, to form various EY005-stearyl-complex particulate formulations. Properties of Kd and binding capacity were calculated for each Y005-stearyl-complex particulate, in dispersal medium (FIG. 24) Formulations of various EY005-prodrug complex particulates provided sustained release of EY005-prodrug in vitro, as compared to comparable formulations of EY005 native peptide, which rapidly released and dumped form formulation (FIG. 25). In in vitro kinetics studies, pilot formulation of prodrug multiphasic colloidal suspension as a bolus implant achieved zero-order (i.e., linear) kinetics of release, achieving the desired durability of drug release of three months, with prodrug present in the dispersal medium and free API present in the in vitro physiologic environment following release of the prodrug from the multiphasic colloidal suspension (FIG. 30).

In in vitro efficacy studies, bolus implant of prodrug multiphasic colloidal suspension was added to RPE cell culture model with endogenous esterases. Cell culture data demonstrated restoration of cytoskeleton, with ˜80% improvement at 21-day timepoint (FIGS. 31A-31D) in association with reversal of cellular mitochondrial dysfunction. This data affirms that prodrug, admixed with complexation agents and incorporated into a dispersal medium to form a stable multiphasic colloidal suspension, can produce sustained release of prodrug at predictable therapeutic levels, and the API remains bioactive upon cleavage of the MTT-prodrug in the surrounding in vitro physiologic environment.

In in vivo kinetics studies, using LC/MS analysis, high retina levels (>300 ng/g) of MTT-prodrug were sustained through 6 weeks for intravitreal injection of a bolus implant of prodrug multiphasic colloidal suspension (H-d-Arg-DMT-Lys-Phe(O)-stearyl payload 1 mg) in rabbit eyes (FIG. 32, EY005-Seteryl release from IVT MitoXR), affirming good in vivo-in vitro correlation for release of prodrug. Recovered bolus had ˜50% residual payload, indicating the bolus implant of prodrug achieves the desired ˜90 day release kinetics of the implant, given zero-order release kinetics.

Further, incorporation of bioactive tetrapeptide API (without prodrug) with the same complexation agent and into the same dispersal medium produced excessive release, or “dump” of the bioactive API in vitro (FIG. 25A, FIG. 30). Additionally, the multiphasic colloidal suspension bolus formulation of native API administered into the vitreous did not produce detectable tissue levels beyond 21 days (FIG. 32, EY005 Peptide Release from Formulated Bolus), indicating excessive release of the native API in vivo as well. Moreover, no residual drug in the recovered bolus, consistent with excessive drug release or “dumping.” Thus, the incorporation of the native unmodified API into the multiphasic colloidal suspension is insufficient to produce sustained release and fails to achieve specifications of an extended release drug delivery system. Importantly, these data affirm and underscore the necessity for the prodrug construct and the specific interaction between prodrug conjugation moiety and complexation agent to form drug substance-complex particulates, for some APIs that do not otherwise form complexes, in order to achieve controlled, durable release of the active API from the multiphasic colloidal suspension XRDDS.

Formulations of the drug substance multiphasic colloidal suspension termed the implant, can be administered in and around the eye, i.e., into the vitreous humor, into the aqueous humor, into the suprachoroidal space, under the retina, under the conjunctiva, beneath Tenon's capsule, into orbital tissue, to produce sustained release of therapeutic levels of drug substance within ocular tissues for desired duration (1 to 12 months).

Formulations of the drug substance multiphasic colloidal suspension may be used to prevent onset or slow progression, modify disease pathobiology, prevent vision loss or improve vision, or prevent onset or improve other destructive or degenerative aspects of ocular conditions and diseases, including dry age-related macular degeneration (AMD), wet AMD, diabetic macular edema (DME), retinal vein occlusion (RVO), and inherited retinal degeneration (IRD), retinal degeneration, traumatic injury, ischemic vasculopathy, acquired or hereditary optic neuropathy, glaucoma, endophthalmitis, retinitis, uveitis, inflammatory diseases of the retina and uveal tract, Fuch's corneal dystrophy, corneal edema, ocular surface disease, dry eye disease, diseases of the conjunctiva, diseases of the periocular tissue, and diseases of the orbit.

The method may be used in conjunction with other treatment modalities including inhibition of vascular endothelial growth factor, complement inhibition, or administration of anti-inflammatory drugs such as corticosteroids.

All of the methods and apparatuses described herein, in any combination, are herein contemplated and can be used to achieve the benefits as described herein.

For example, described herein are compositions of a multiphasic colloidal suspension comprising a drug substance and one or more complexation agents, admixed in a dispersal medium. The one or more complexation agents may be a chemical substance formulated as an irregular shaped particulate that is capable of forming drug substance-complex particulates by noncovalent, reversible binding to the drug substance, and that is one of: a fatty acid, an organic compound that can form a keto-enol tautomer, a charged phospholipid, a charged protein, a ribonucleic acid, and a polysaccharide.

For example, the complexation agent may be a fatty acid comprising: a carboxylic acid with an aliphatic chain with chemical formula of CH3(CH2)ₙCOOH where n is equal to between 4 and 30, which is either saturated or unsaturated, and is a salt or an ester, and which includes one or more of: magnesium palmitate, magnesium stearate, calcium palmitate, calcium stearate.

The particulate complexation agent may be an organic compound that can form a keto-enol tautomer and is capable of undergoing chemical equilibrium between a keto form consisting of a ketone or an aldehyde, and an enol form and includes one of: a phenol compound, a tocopherol compound, a quinone compound, a ribonucleic acid compound. In some examples the particulate complexation agent is a charged phospholipid and is one or more of: an anionic phospholipid, lecithin, phosphatidylcholine, phosphatidylethanolamine, phosphatidylserine, sphingomyelin, a synthetic phospholipid with a positive charge, and DLin-MC3-DMA. The particulate complexation agent may be a charged protein that is either positive or negative and is one or more of: albumin, a synthetic polypeptide, a plasma protein, alpha2-macroglobulin, fibrin, and collagen. The particulate complexation agent may be a ribonucleic acid, a biopolymer macromolecule comprising nucleotides comprising a 5-carbon sugar, a phosphate group, and a nitrogenous base. The particulate complexation agent may be a polysaccharide, comprising a long chain polymeric carbohydrates comprising monosaccharide units bound together by glycosidic linkages, and includes one or more of: a ringed polysaccharide molecule, cyclodextrin, and a clathrate.

The drug substance may form noncovalent complexes with the particulate complexation agent, and may comprise one of: a small molecule, a small polypeptide, a protein, an aptamer, a nucleic acid drug, a hydrophobic chemical, and a hydrophilic chemical. In some examples the drug substance is a prodrug of formula (I): R′—R (I), where R′ is any active pharmaceutical ingredient (API) that is covalently linked via cleavable bond to R, a conjugation moiety that forms noncovalent complexes with one of five classes of complexation agents, and the covalent bond linking R′ and R may be removed by enzymatic cleavage, catalysis, hydrolysis, or other reaction to yield free API R′ and conjugation moiety R, where R is selected from: a C4-C30 lipid moiety (fatty acid or fatty alcohol), an C4-C30 straight-chain or branched aliphatic moiety, a 2-mer to 30-mer peptide moiety, a pegylated moiety, or a carbohydrate moiety. The cleavable covalent bond may comprise one of: an ester bond, a hydrazone bond, an imine bond, a disulfide bond, a thioester bond, a thioether bond, a phosphate ester bond, a phosphonate ester bond, a boronate ester bond, an amide bond, a carbamate ester bond, a carboxylate ester bond, and a carbonate ester bond.

The conjugation moiety may be a fatty alcohol, with or without a preceding linker moiety, that includes one or more of: tert-butyl alcohol, tert-amyl alcohol, 3-methyl-3-pentanol, 1-heptanol (enanthic alcohol), 1-octanol (capryl alcohol), 1-nonanol (pelargonic alcohol), 1-decanol (decyl alcohol, capric alcohol), undecyl alcohol (1-undecanol, undecanol, hendecanol), dodecanol (1-dodecanol, lauryl alcohol), tridecyl alcohol (1-tridecanol, tridecanol, isotridecanol), 1-tetradecanol (myristyl alcohol), pentadecyl alcohol (1-pentadecanol, pentadecanol), 1-hexadecanol (cetyl alcohol), cis-9-hexadecen-1-ol (palmitoleyl alcohol), heptadecyl alcohol (1-n-heptadecanol, heptadecanol), 1-octadecanol (stearyl alcohol), 1-octadecenol (oleyl alcohol), 1-nonadecanol (nonadecyl alcohol), 1-eicosanol (arachidyl alcohol), 1-heneicosanol (heneicosyl alcohol), 1-docosanol (behenyl alcohol), cis-13-docosen-1-ol (erucyl alcohol), 1-tetracosanol (lignoceryl alcohol), 1-pentacosanol, 1-hexacosanol (ceryl alcohol), 1-heptacosanol, 1-octacosanol (montanyl alcohol, cluytyl alcohol), 1-nonacosanol, 1-triacontanol (myricyl alcohol, melissyl alcohol).

The conjugation moiety may be a fatty acid, with or without a preceding linker moiety, that comprises one or more of: Tetradecanoic acid, pentadecanoic acid, (9Z)-hexadecenoic acid, Hexadecanoic acid, Heptadecanoic acid, Octadecanoic acid, (9Z,12Z)-octadeca-9,12-dienoic acid, (9Z,12Z,15Z)-octadeca-9,12,15-trienoic acid, (6Z,9Z,12Z)-octadeca-6,9,12-trienoic acid, (5E,9E,12E)-octadeca-5,9,12-trienoic acid, (6Z,9Z,12Z,15Z)-octadeca-6,9,12,15-tetraenoic acid, (Z)-octadec-9-enoic acid, (11E)-octadec-11-enoic acid, (E)-octadec-9-enoic acid, nonadecanoic acid, and eicosanoic acid.

In some examples, R is a 2-mer to about a 30-mer peptide moiety comprising natural or synthetic amino acids, which is one of: anionic, cationic, or neutral, with or without a preceding linker moiety, that includes one or more of: poly-glutamate, poly-aspartate, or a combination of glutamate and aspartate; poly-arginine, poly-lysine, poly-histidine, a combination of arginine and lysine, a combination of arginine and histidine, a combination of histidine and lysine, or a combination of arginine, histidine, and lysine; peptide moiety has one or more PEGylation sites for addition of polyethylene glycol (PEG) groups; peptide moiety has one or more sites for modification by addition of sugar or carbohydrate molecules, including glycosylation. In some examples, R is one of: a polyethylene glycol (PEG) polymer, a pegylated peptide, or pegylated succinate including PEG polymers of linear, branched, Y-shaped, or multi-arm geometries. In some examples R is a carbohydrate moiety comprising a carbohydrate of 2 to 20 sugars, with or without a preceding linker moiety, comprising one or more of: glucose, galactose, lactose, mannose, ribose, fucose, N-acetylgalactosamine, N-acetylglucosamine, N-acetyleneuraminic acid, or an epimer or derivative of glucose, galactose, lactose, mannose, ribose, fucose, N-acetylgalactosamine, N-acetylglucosamine, and N-acetyleneuraminic acid.

The R′ may be an API, and R is a linker or multimerization domain which is covalently linked to multiple API to form dimers or multimers of the prodrug and n is equal to 2 to about 100, and R is one of: a PEG, a PEG polymer, polyvinyl alcohol (PVA), or peptide.

In any of these compositions the dispersal medium may be a liquid oil capable of forming multiphasic colloidal suspension, comprising a hydrophobic oil comprising at least one of: saturated fatty acid methyl esters, unsaturated fatty acid methyl esters, saturated fatty acid ethyl esters, unsaturated fatty acid ethyl esters.

The saturated fatty acid methyl esters may comprise one or more of: methyl acetate, methyl propionate, methyl butyrate, methyl pentanoate, methyl hexanoate, methyl heptanoate, methyl octanoate, methyl nonanoate, methyl decanoate, methyl undecanoate, methyl dodecanoate (methyl laurate), methyl tridecanoate, methyl tetradecanoate, methyl 9(Z)-tetradecenoate, methyl pentadecanoate, methyl hexadecanoate, methyl heptadecanoate, methyl octadecenoate, methyl nonadecanoate, methyl eicosanoate, methyl heneicosanoate, methyl docosanoate, methyl tricosanoate, and others.

The unsaturated fatty acid methyl esters may comprise: methyl 10-undecenoate, methyl 11-dodecenoate, methyl 12-tridecenoate, methyl 9(E)-tetradecenoate, methyl 10(Z)-pentadecenoate, methyl 10(E)-pentadecenoate, methyl 14-pentadecenoate, methyl 9(Z)-hexadecenoate, methyl 9(E)-hexadecenoate, methyl 6(Z)-hexadecenoate, methyl 7(Z))-hexadecenoate, methyl 11(Z)-hexadecenoate.

The saturated fatty acid ethyl esters may comprise: ethyl acetate, ethyl propionate, ethyl butyrate, ethyl pentanoate, ethyl hexanoate, ethyl heptanoate, ethyl octanoate, ethyl nonanoate, ethyl decanoate, ethyl undecanoate, ethyl dodecanoate (ethyl laurate), ethyl tridecanoate, ethyl tetradecanoate, ethyl 9(Z)-tetradecenoate, ethyl pentadecanoate, ethyl hexadecanoate, ethyl heptadecanoate, ethyl octadecenoate, ethyl nonadecanoate, ethyl eicosanoate, ethyl heneicosanoate, ethyl docosanoate, ethyl tricosanoate.

The unsaturated fatty acid ethyl esters may comprise: ethyl 10-undecenoate, ethyl 11-dodecenoate, ethyl 12-tridecenoate, ethyl 9(E)-tetradecenoate, ethyl 10(Z)-pentadecenoate, ethyl 10(E)-pentadecenoate, ethyl 14-pentadecenoate, ethyl 9(Z)-hexadecenoate, ethyl 9(E)-hexadecenoate, ethyl 6(Z)-hexadecenoate, ethyl 7(Z))-hexadecenoate, ethyl 11(Z)-hexadecenoate.

Also described herein are composition of a multiphasic colloidal suspension comprising a drug substance and one or more complexation agents, admixed in a dispersal medium having a release profile of one or more phases of drug release, wherein the one or more complexation agents is formulated as an irregular-shaped particulate that forms drug substance-complex particulates by noncovalent, reversible binding to the drug substance, and is one of: a fatty acid, an organic compound that can form a keto-enol tautomer, a charged phospholipid, a charged protein, a ribonucleic acid, and a polysaccharide, further wherein the drug substance comprises one of: a small molecule, a small polypeptide, a protein, an aptamers, a nucleic acid drug, a hydrophobic chemical, and a hydrophilic chemical; further wherein the dispersal medium is a hydrophobic liquid oil comprising at least one of: saturated fatty acid methyl esters, unsaturated fatty acid methyl esters, saturated fatty acid ethyl esters, unsaturated fatty acid ethyl esters.

For example, a composition of a multiphasic colloidal suspension may comprise a drug substance and one or more complexation agents, admixed in a dispersal medium having a release profile of one or more phases of drug release, wherein the one or more complexation agents is formulated as an irregular-shaped particulate that forms drug substance-complex particulates by noncovalent, reversible binding to the drug substance, and is one of: a fatty acid, an organic compound that can form a keto-enol tautomer, a charged phospholipid, a charged protein, a ribonucleic acid, and a polysaccharide; further wherein the dispersal medium is a hydrophobic liquid oil comprising at least one of: saturated fatty acid methyl esters, unsaturated fatty acid methyl esters, saturated fatty acid ethyl esters, unsaturated fatty acid ethyl esters.

In some examples, a composition of a multiphasic colloidal suspension comprises a drug substance and one or more complexation agents, admixed in a dispersal medium having a release profile of one or more phases of drug release, wherein the one or more complexation agents is formulated as an irregular-shaped particulate that forms drug substance-complex particulates by noncovalent, reversible binding to the drug substance, and is one of: a fatty acid, an organic compound that can form a keto-enol tautomer, a charged phospholipid, a charged protein, a ribonucleic acid, and a polysaccharide, further wherein the drug substance comprises one of: a small molecule, a small polypeptide, a protein, an aptamers, a nucleic acid drug, a hydrophobic chemical, and a hydrophilic chemical; further wherein the dispersal medium is a hydrophobic liquid oil comprising at least one of: saturated fatty acid methyl esters, unsaturated fatty acid methyl esters, saturated fatty acid ethyl esters, unsaturated fatty acid ethyl esters.

Also described herein are methods of designing a composition of a multiphasic colloidal suspension comprising a drug substance and one or more complexation agents admixed in a dispersal medium, to meet a prespecified release rate and amount of drug substance. For example, the method may include: varying the ratios and amounts of different drug substance-complex particulates with different binding capacity and Kd. The method may include varying the ratios and amounts of different drug substance-complex particulates with different binding capacity and Kd comprises adding drug substance-complex particulates with high binding capacity and high Kd, indicating low avidity of drug substance to the particulate complexation agent, to create a short-term increased rate of release, or initial burst. Any of these methods may include extending the duration of release of the drug substance from an implant by adding drug substance-complex particulates with high binding capacity and low Kd, indicating high avidity of drug substance to the particulate complexation agent. Any of these methods may include formulating the drug substance-multiphasic colloidal suspension for ocular injection as one of: a flowable bolus implant, an erodible or non-bioerodible tube implant filled with drug substance-multiphasic colloidal suspension, or a drug substance-multiphasic colloidal suspension fashioned into a solid mold of a specific size and shape and configured for implantation.

Also described herein are methods of treatment using any of these compositions. For example, described herein are methods of treating a disorder and disease of the eye, wherein the drug substance-multiphasic colloidal suspension is administered in and around the eye, into one of the following tissue compartments: vitreous humor, into the aqueous humor, into the suprachoroidal space, under the retina, under the conjunctiva, beneath Tenon's capsule, or into orbital tissue, to produce sustained release of therapeutic levels of drug substance within ocular tissues for one or more months.

For example, a method of treatment of vision loss in a subject, by intravitreal or periocular injections of formulations of extended release drug delivery system that produces high sustained retina and retinal pigment epithelium (RPE) tissue levels of active drug, may include: delivering a drug substance that is a prodrug combined with the extended-release drug delivery system into the subject's eye at a treatment start; and cleaving, by action of an esterase or bioactive enzyme in the subject's eye, the prodrug to release the active pharmaceutical ingredient (API) of the prodrug into the eye during a first phase at a burst phase release rate; and cleaving, by action of the esterase or bioactive enzyme, the prodrug to release the API into the eye during a second phase at a steady-state dose rate, wherein the burst phase rate is greater than the steady state release rate, further wherein the first phase extends from the treatment start for about 2-6 weeks and the subsequent phases extend from an end of the first phase for one or more months.

For example, a method of preventing onset of atrophy or slowing progression of atrophy of the neurosensory retina and/or retinal pigment epithelium (RPE) in a subject, by intravitreal or periocular injections of formulations of extended release drug delivery system that produce high sustained retina and RPE tissue levels of active drug, may include: delivering a drug substance that is a prodrug of an active pharmaceutical ingredient (API) combined with an extended release drug delivery system into the subject's eye at a treatment start; and cleaving, by action of an esterase or bioactive enzyme in the subject's eye, the prodrug to release the API into the eye during a first phase at a burst phase release rate; and cleaving, by action of the esterase or bioactive enzyme, the prodrug to release the API into the eye during a second phase at a steady-state dose rate, wherein the burst phase rate is greater than the steady state release rate, further wherein the first phase extends from the treatment start for about 2-6 weeks and the subsequent phase extends from an end of the first phase for one or more months.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 illustrates the components of the multiphasic colloidal suspension, wherein either a drug substance (100) (defined as various small polypeptides, proteins, aptamers, other nucleic acid drugs, hydrophobic chemicals, hydrophilic chemicals, and other chemical compounds used for therapeutic purposes) or a prodrug (101) (e.g., any active pharmaceutical ingredient (103) linked via cleavable covalent bond to one of five classes of conjugation moieties (105)) is added and admixed to (107) one or more complexation agents, in a hydrophobic dispersal medium. Collectively, these form the (109) multiphasic colloidal suspension extended release drug delivery system, which may be administered, e.g., in and around the eye in various formulations to achieve treatment durations of 1-12 months, for the treatment of various diseases of the eye, periocular tissue, and orbit.

FIG. 2A-2E illustrates the approach of custom design of a formulation for specific pharmacokinetics release profile by mathematical formula using the multiphasic colloidal suspension extended-release drug delivery system, including in this specific example, one method of configuring a two-phase release profile (FIG. 2A) for release of a drug substance as described herein.

FIGS. 3A-3C illustrates three different potential release kinetic profiles of a drug substance from the multiphasic colloidal suspension, including (FIG. 3A) one-phase (zero-order) release kinetics, (FIG. 3B) two-phase release kinetics, and (FIG. 3C) three-phase release kinetics.

FIGS. 4A-4F illustrate complexation of various fluorescent small molecules with magnesium stearate, assayed using fluorescence microscopy. FIG. 4A shows Magnesium stearate alone shows low intrinsic fluorescence. FIG. 4B shows Magnesium stearate incubated with FITC-labeled EY005 native peptide (H-d-Arg-DMT-Lys-Phe). FITC-labeled EY005 native peptide alone showed minimal complexation with magnesium stearate as reflected by minimal fluorescent labeling of particulates. FIG. 4C shows Magnesium stearate incubated with FITC-labeled EY005-stearate prodrug (H-d-Arg-DMT-Lys-Phe(O)-stearyl). Complexation of EY005-stearyl prodrug with magnesium stearate was evident as moderate fluorescence of imaged magnesium stearate particulates. FIG. 4D shows that treating FITC-labeled EY005-stearyl prodrug (wherein the FITC labeled the peptide and not the stearyl conjugation moiety) complexed with magnesium from sample C with carboxyesterase (0.1 μg/mL) reduced levels of fluorescence, demonstrating that the complexation was specifically mediated by the stearyl conjugation moiety. FIG. 4E shows Magnesium stearate incubated with the fluorescent C12 lipid compound. Strong complexation of C12 lipid compound with magnesium stearate was evident as bright fluorescence of imaged magnesium stearate particulates. FIG. 4F shows Magnesium stearate incubated with fluorescent cationic small molecule, which complexed strongly with magnesium stearate as seen by bright fluorescence of magnesium stearate particulates.

FIGS. 5A-5F illustrate complexation of various fluorescent small molecules with albumin, assayed using fluorescence microscopy. In FIG. 5A, Albumin alone shows low intrinsic fluorescence.

FIG. 5B shows Albumin incubated with FITC-labeled EY005 native peptide (H-d-Arg-DMT-Lys-Phe). FITC-labeled EY005 native peptide alone showed minimal complexation with albumin as reflected by negative staining of albumin crystals surrounded by generalized fluorescence from dissolved FITC-labeled EY005 native peptide. FIG. 5C shows Albumin incubated FITC-labeled EY005-stearate prodrug (H-d-Arg-DMT-Lys-Phe(O)-stearyl). Strong complexation of EY005-stearyl prodrug with albumin was evident as bright fluorescence of imaged albumin crystals. In FIG. 5D, treating FITC-labeled EY005-stearyl prodrug (wherein the FITC labeled the peptide and not the stearyl conjugation moiety) complexed with albumin from sample C with carboxyesterase (0.1 μg/mL) reduced levels of fluorescence demonstrating that the complexation was specifically mediated by the stearyl conjugation moiety. FIG. 5E shows Albumin incubated with the fluorescent C12 lipid compound. Strong complexation of fluorescent C12 lipid compound with albumin was evident as bright fluorescence of imaged albumin particulates. FIG. 5F shows Albumin incubated with fluorescent cationic small molecule, which complexed moderately with albumin as seen by moderate fluorescence of albumin particulates.

FIGS. 6A-6F illustrate complexation of various fluorescent small molecules with cyclodextrin gamma, assayed using fluorescence microscopy. In FIG. 6A, Cyclodextrin alone shows low intrinsic fluorescence. FIG. 6B shows Cyclodextrin incubated with FITC-labeled EY005 native peptide (H-d-Arg-DMT-Lys-Phe). FITC-labeled EY005 native peptide alone showed minimal complexation with cyclodextrin as reflected by minimal increase in fluorescence above that of cyclodextrin alone. FIG. 6C shows Cyclodextrin incubated FITC-labeled EY005-stearate prodrug (H-d-Arg-DMT-Lys-Phe(O)-stearyl. Complexation of EY005-stearyl prodrug with cyclodextrin was evident as moderate fluorescence of imaged prodrug-cyclodextrin particulates. FIG. 6D shows treating FITC-labeled EY005-stearyl prodrug (wherein the FITC labeled the peptide and not the stearyl conjugation moiety) complexed with cyclodextrin from sample C with carboxyesterase (0.1 μg/mL) reduced levels of fluorescence demonstrating that the complexation was specifically mediated by the stearyl conjugation moiety. FIG. 6E shows Cyclodextrin incubated with a fluorescent C12 lipid compound. Complexation of fluorescent C12 lipid compound with cyclodextrin was evident as moderate fluorescence of imaged cyclodextrin particulates. In FIG. 6F, Cyclodextrin was incubated with fluorescent cationic small molecule, which complexed more strongly with cyclodextrin creating brightly fluorescent particulates.

FIGS. 7A-7F illustrate complexation of various fluorescent small molecules with lecithin, assayed using fluorescence microscopy. In FIG. 7A, Lecithin alone shows low intrinsic fluorescence. FIG. 7B shows Lecithin incubated with FITC-labeled EY005 native peptide (H-d-Arg-DMT-Lys-Phe). FITC-labeled EY005 native peptide showed minimal complexation with lecithin as reflected by minimal increase in fluorescence above that of lecithin alone. FIG. 7C shows Lecithin incubated with FITC-labeled EY005-stearate prodrug (H-d-Arg-DMT-Lys-Phe(O)-stearyl). Complexation of EY005-stearyl prodrug with lecithin was evident as bright fluorescence of all lecithin samples. In FIG. 7D, treating FITC-labeled EY005-stearyl prodrug (wherein the FITC labeled the peptide and not the stearyl conjugation moiety) complexed with lecithin from sample C with carboxyesterase (0.1 μg/mL) reduced levels of fluorescence demonstrating that the complexation was specifically mediated by the stearyl conjugation moiety. FIG. 7E shows Lecithin incubated with a fluorescent C12 lipid compound. Complexation of fluorescent C12 lipid compound with lecithin was evident as bright fluorescence of all lecithin samples. FIG. 7F shows Lecithin incubated with fluorescent cationic small molecule, which showed minimal complexation with lecithin as evidence by only dim fluorescence in lecithin samples.

FIGS. 8A-8F illustrate complexation of various fluorescent small molecules with silica microbeads, particulate that does not serve as a complexation agent, assayed using fluorescence microscopy. In FIG. 8A, Silica microbeads alone were imaged as a negative control and showed minimal intrinsic fluorescence. FIG. 8B shows Silica microbeads incubated with FITC-labeled EY005 native peptide (H-d-Arg-DMT-Lys-Phe). FITC-labeled EY005 native peptide complexed with silica microbeads creating fluorescent round figures, which dissipated on addition of the multiphasic colloidal suspension to an ocular physiologic environment, indicating low avidity of complexation. FIG. 8C shows Silica microbeads incubated with FITC-labeled EY005-stearyl prodrug (H-d-Arg-DMT-Lys-Phe(O)-stearyl). There was no evidence of complexation of EY005-stearyl prodrug with silica microbeads. In FIG. 8D, treating FITC-labeled EY005-stearyl prodrug (wherein the FITC labeled the peptide and not the stearyl conjugation moiety) complexed with silica microbeads from sample C with carboxyesterase (0.1 μg/mL) did not alter extremely low levels of fluorescence. FIG. 8E shows Silica microbeads incubated with a fluorescent C12 lipid compound. The dim fluorescence on the surface of silica microbeads suggests minimal complexation with fluorescent C12 lipid compound. FIG. 8F shows Silica microbeads incubated with fluorescent cationic small molecule, which complexed extensively with silica microbeads creating brightly fluorescent round figures.

FIG. 9 demonstrates examples of kinetics of release by daily release rate of various formulations of fluocinolone acetonide (F17 FA), dexamethasone free base (F10 DEX), and dexamethasone phosphase (F1 DEX PHOS).

FIGS. 10A-10F illustrates fluorescent microbeads (3 μm and 10 μm) in water. Very rapid settling occurs with upper regions of the mixture showing very few microbeads and bottom levels of the mixture showing very dense microbeads. Thus, this mixture does not behave as a multiphasic colloidal suspension since particulates are not uniformly dispersed.

FIGS. 11A-11F illustrates fluorescent microbeads (3 μm and 10 μm) in silicone oil. Very rapid settling occurs with upper regions of the mixture showing few microbeads and bottom levels of the mixture showing very dense microbeads. Thus, this mixture does not behave as a multiphasic colloidal suspension since particulates are not uniformly dispersed.

FIGS. 12A-12F illustrates fluorescent microbeads (3 μm and 10 μm) in methyl laurate. Beads remain uniformly dispersed without evidence of settling or migration. Thus, methyl laurate is an effective dispersal medium since this forms a stable multiphasic colloidal suspension.

FIG. 13A-13F illustrates fluorescent microbeads (3 μm and 10 μm) in 2% gelatin. Beads remain uniformly dispersed without evidence of settling or migration, indicating formation of a colloidal suspension.

FIG. 14A-14F illustrates fluorescent microbeads (3 μm and 10 μm) in 2% gelatin, treated with collagenase. Following treatment, beads rapidly settle with higher proportion of beads in the lower wells, indicating that that gelatin cannot serve as a dispersal medium in an ocular physiologic environment with abundant enzymes that will degrade gelatin. Thus, this does not represent a stable multiphasic colloidal suspension, and 2% gelatin is not an effective dispersal medium.

FIG. 15 shows Table 1 illustrates binding capacity (μg fluocinolone acetonide complexed per mg complexation agent) and Kd (unbound-bound ratio) for fluocinolone acetonide added to methyl laurate and admixed with various complexation agents, with centrifuge and pulldown of particulates after 1 hour of incubation. The quantity of fluocinolone acetonide complexed with each complexation agent was determined by HPLC. These data demonstrated variable degrees of complexation with each class of complexation agent.

FIG. 16A illustrates how different representative formulations of drug and complexation agent(s) produce specific and different release kinetics in vitro, wherein the drug release kinetics of each formulation are designed and tuned in a predictable fashion by varying the ratios of, in this example, two different complexation agents. Formulation 1 depicts a shorter duration release profile (i.e., 120 days), while formulation 2 depicts a two-phase release profile with longer duration (i.e., 210 days).

FIG. 16B illustrates how the effect of varying proportions of complexation agents to time release kinetics, as described herein, for a given drug payload. As described in FIG. 2, the measured drug release kinetics from individual drug-complexes can be utilized to determine a predicted target release kinetics for blends of two or more drug-complexes, which can be confirmed experimentally by in vitro release studies as in FIG. 9

FIGS. 17A and 17B illustrate good in vitro to in vivo correlation for two different formulations of fluocinolone acetonide in the multiphasic colloidal suspension. The depicted curves reflect in vitro release profiles, while the individual colored points, circled, represent in vivo release data from rabbit eyes.

FIG. 18 shows Table 2, illustrates binding capacity (μg fluocinolone acetonide complexed per mg complexation agent) and Kd (unbound-bound ratio) for dexamethasone phosphate added to methyl laurate and admixed with various complexation agents, with centrifuge and pulldown of particulates after 1 hour of incubation. The quantity of dexamethasone phosphate complexed with each complexation agent was determined by HPLC. These data demonstrated variable degrees of complexation with each class of complexation agent.

FIG. 19 illustrates how physicochemical properties of the drug substance influence interactions with complexation properties. Hydrophilic drug substance dexamethasone phosphate (DexPh) and hydrophobic drug substance fluocinolone acetonide (FA) (at same payloads) was each admixed with same particulate complexation agents (magnesium stearate and tocopherol) and dispersal medium (methyl laurate) (circles for DexPh, triangles bottom curve for FA). Formulation of DexPh demonstrated a rapid and excessive release, or “dump” of DexPh. The addition of a different complexation agent, lecithin, with reduction in ratios of other complexation agents, for a given payload (triangles, middle curve) altered the kinetic release profile to minimize dump of DexPh and provide more desirable sustained release profile.

FIG. 20 shows Table 3, illustrating binding capacity (μg fluocinolone acetonide complexed per mg complexation agent) and Kd (unbound-bound ratio) for sunitinib malate added to dispersal medium and admixed with various complexation agents, with centrifuge and pulldown of particulates after 1 hour of incubation. The quantity of sunitinib malate complexed with each complexation agent was determined by HPLC. These data demonstrated variable degrees of complexation with each class of complexation agent.

FIG. 21 illustrates that complexation can also be assessed by colorimetric analysis for certain compounds. Sunitinib is a brightly colored, yellow compound. To evaluate complexation, various complexation agents were incubated with a solution of sunitinib maleate for one hour. Complexation agents were then rinsed five times to remove all free sunitinib. Following rinsing, complexation agents were imaged to evaluate relative degrees of sunitinib complexation. As can be seen, colorimetric changes occur to varying degrees for each complexation agent suggesting variable levels of complexation with sunitinib.

FIG. 22 illustrate formulations (as multiphasic colloidal suspension in bio-erodible tube) and drug substance release of sunitinib malate, with detectable drug levels in retina tissue of rabbits of low and high dose implants at multiple time points and durability of release of each. Tissue levels for the high dose implant remained consistently above IC₉₀ levels for sunitinib.

FIG. 23 illustrate formulations (as multiphasic colloidal suspension as flowable bolus) and drug substance release of axitinib, with detectable drug levels in retina tissue of rabbits of implants at multiple time points and durability of release. Tissue levels remained consistently above IC₉₀ levels of axitinib.

FIG. 24 is Table 4, which illustrates binding capacity (μg fluocinolone acetonide complexed per mg complexation agent) and Kd (unbound-bound ratio) for EY005-stearyl prodrug added to dispersal medium and admixed with various complexation agents, with centrifuge and pulldown of particulates after 1 hour of incubation. The quantity of centrifuge complexed with each complexation agent was determined by HPLC. These data demonstrated variable degrees of complexation with each class of complexation agent.

FIG. 25A shows data from an accelerated in vitro release assay in which EY005 MTT was formulated with various complexation agents using methyl laurate as a dispersal medium. In all cases, all EY005 was rapidly released, in some cases within hours, and in all cases by day 3.

FIG. 25B shows data from an accelerated in vitro release assay in which EY005-stearyl prodrug was formulated with various complexation agents using methyl laurate as a dispersal medium. Formulations with no complexation agent show rapid release of EY005 into the media. A formulation with silica microbeads, which does not complex with EY005-stearyl prodrug also shows rapid release of EY005 into the media. By contrast, formulations with other complexation agents demonstrate sustained release of EY005 at varying rates. Of particular note, when magnesium stearate and albumin are both used as complexation agents in the same formulation, EY005 is released at a rate intermediate between that of formulations using either complexation agent alone.

FIG. 26 schematically illustrates one example of a mitochondrial-targeted tetrapeptide EY005 (103), which when linked to one of several classes of conjugation moieties (105), comprises a mitochondrial-targeted peptide prodrug (101). This mitochondrial-targeted peptide prodrug is admixed with selected complexation agents in dispersal medium to form the multiphasic colloidal suspension in bolus formulation (107), which can be injected into the vitreous of eye (109), as part of an intravitreal (IVT) extended release drug delivery system.

FIG. 27A is an example of a prodrug of the EY005 mitochondrial targeted tetrapeptide including a stearyl alcohol or octadecyl moiety linked by ester bond to the mitochondrial targeted tetrapeptide.

FIG. 27B is an example of a prodrug of the EY005 mitochondrial targeted tetrapeptide including a peptide motif (e.g., an anionic tri-Glu peptide) and linker moiety linked via ester bond to EY005.

FIG. 27C is an example of a prodrug of the EY005 mitochondrial targeted tetrapeptide including a peptide motif (e.g., a cationic tri-Arg peptide) and linker moiety linked via ester bond to EY005.

FIG. 27D is an example of a prodrug of the EY005 mitochondrial targeted tetrapeptide including a polyethylene glycol (PEG) linked by ester bond to EY005.

FIGS. 28A-28C demonstrates cleavage of ester based EY005-stearyl prodrug by carboxyesterase and by spontaneous hydrolysis. FIG. 28A shows baseline HPLC analysis of EY005-stearyl prodrug (top tracing) and EY005 MTT (bottom tracing). EY005-stearyl was incubated at 37° C. in vitro with carboxyesterase (0.1 μg/mL), to simulate the ocular physiologic environment and the type of esterase that is readily abundant within the vitreous. Incubation of EY005-stearyl with carboxyesterase produced rapid cleavage of the prodrug ester bond, releasing EY005, as evident by disappearance of the EY005-stearyl prodrug peak and appearance of the EY005 peak on high performance liquid chromatography (FIG. 28B). Upon addition of EY005-stearyl prodrug to phosphate-buffered saline solution at 37° C. without esterase, the ester bond of the EY005-stearyl prodrug cleaves more slowly by hydrolysis (FIG. 28C). After 6 hours, partial cleavage of EY005-stearyl prodrug to EY005 MTT is noted.

FIG. 29A shows an in vitro culture model of dry AMD in which RPE cells possessing endogenous esterases were exposed to hydroquinone (HQ) to induce mitochondrial dysfunction. Mitochondrial dysfunction is manifest as increased flavoprotein autofluorescence (upper panels) and as dysmorphology of the actin cytoskeleton (lower panels). EY005-stearyl (5 μM) effectively reversed HQ-induced mitochondrial dysfunction in RPE cells (as depicted by reduced cellular flavoprotein-autofluorescence and normalized actin cytoskeleton dysfunction), with efficacy equivalent to treatment with EY005 native peptide (5 μM). EY005-stearyl was also preincubated with carboxyesterase (0.1 g/mL) in separate media. Recovered media containing cleaved EY005 (5 μM) was added to this RPE cellular model of mitochondrial dysfunction, and this was similarly effective and equipotent to EY005 native peptide for the reversal of RPE mitochondrial dysfunction.

FIG. 29B shows quantification of flavoprotein autofluorescence (FP-AF) from at least 3 replicates of each condition represented in FIG. 29A. Both EY005-stearyl and esterase-cleaved EY005-stearyl show potency equal to the native EY005 peptide.

FIG. 29C shows quantification of actin cytoskeletal dysmorphology from at least 3 replicates of each condition represented in FIG. 29A. Both EY005-stearyl and esterase-cleaved EY005-stearyl show potency equal to the native EY005 peptide.

FIG. 30 shows in vitro pharmacokinetics of a pilot formulation of Mito XR (triangles). Mito XR achieved zero-order (i.e., linear) kinetics of EY005 bioactive tetrapeptide release, achieving the desired durability of drug release of three months, with free bioactive MTT within the dispersal medium released from the implant into the ocular physiologic environment. By contrast, identically formulated EY005 native peptide (circles) had extremely rapid release and did not provide desired durability of drug release.

FIGS. 31A-31C depicts an in vitro culture model of dry AMD in which RPE cells possessing endogenous esterases were exposed to hydroquinone (HQ) to induce mitochondrial dysfunction. Mitochondrial dysfunction is manifest as dysmorphology of the actin cytoskeleton. In these efficacy studies, a bolus implant of Mito XR (EY005-stearyl formulated in multiphasic colloidal suspension) was added to RPE cell culture model of dry AMD with endogenous esterases present. Treatment with Mito XR implant resulted in reversal of mitochondrial dysfunction and concurrent restoration of actin cytoskeletal morphology.

FIG. 31D shows a graphical representation of data from FIGS. 31A-31C. Cultured RPE cells were graded for severity of actin cytoskeletal dysmorphology in control, HQ-exposed cells and HQ-exposed cells treated with Mito XR. Results from at least 3 replicates were quantified. Cultures treated with Mito XR demonstrate an 80% reduction in severity of RPE cell actin cytoskeletal dysmorphology compared to control, HQ-exposed cells.

FIG. 32 shows superior in vivo pharmacokinetics of EY005-stearyl prodrug formulated as Mito XR. Rabbits were injected with intravitreal Mito XR implant containing formulated EY005-stearyl prodrug (EY005-stearyl release from IVT MitoXR) or with identical bolus formulation containing EY005 native peptide (EY005 peptide release from formulated bolus). EY005-stearyl prodrug formulated as Mito XR showed retinal tissue concentrations exceeding the EC₅₀ for reversal of mitochondrial dysfunction. These therapeutic drug levels were sustained through the 7-week timepoint, at which recovered Mito XR implants still contained 50% payload, indicating that this formulation will achieve desired 90-day durability. By contrast, native EY005 peptide was rapidly released with tissue concentrations nearly undetectable by week 3.5. Recovered implants contained no EY005 native peptide suggesting rapid dumping of drug in vivo.

FIGS. 33A-33C illustrate examples of delivery forms or modalities, for delivering implants of any of the multiphasic colloidal suspension extended-release drug delivery systems, which may be comprising one or more complexation agents noncovalently complexed with a prodrug of a mitochondrial-targeted tetrapeptide. FIG. 33A shows an example of a bolus injection in which the extended-release drug delivery system material is formulated as an injectable liquid bolus. FIG. 33B is an example in which the MTT-prodrug multiphasic colloidal suspension is formulated as a tube implant with a biodegradable outer sleeve/tubing filled with multiphasic colloidal suspension containing prodrug and complexation agents. FIG. 33C is an example in which the extended-release drug delivery system material is molded into a particular shape with solid state for implantation.

FIG. 33D illustrates two methods of injecting formulations of the multiphasic colloidal suspension into the eyes, either as a bolus injection or a tube implant.

FIGS. 34A-34B illustrates the effect of varying the inner diameter/radius of the open ends of the injectable tube implant modality of the complexation-based XRDDS. Release rate is decreased predictably in proportion to the radius/diameter of the tube end. FIG. 34A illustrates the dimensions of an end of a tube (depot). FIG. 34B is a graph showing the release rate over time (days) for two examples of a given extended-release corticosteroid formulation, each released from tubes of different inner diameter/radius (r). As predicted, the formulation within PE10 tube with lower r value has lower release rate as compared to the formulation within PE50 with the higher r value.

FIG. 34C illustrates the use of an ultra-thin wall 25 gauge needle appropriate for intravitreal injection, with bioerodible or non-bioerodible tubes (depots) for release of extended-release corticosteroid formulations, emerging from the lumen of the 25 gauge needle.

FIGS. 35A-35D illustrates that the composition of a bioerodible tube that can permit 2-phase drug release profile (FIG. 35B) or a composition of a bioerodible tube that can permit a 3-phase drug release profile, enabling accelerated release at later time points. In FIG. 35A the bioerodible tube is a tube of PLGA composition 82 L/18 G tube, showing the tube is intact after all of the drug has been released. FIG. 35B is a graph showing the resulting 2-phase kinetics profile for the tube of FIG. 35A, filled with an extended-release corticosteroid formulation. FIG. 35C shows a bioerodible tube has PLGA composition 80 L/20 G tube that degrades before all of the drug has been released, resulting in a 3-phase release profile, when releasing an extended-release corticosteroid formulation as shown in FIG. 35D.

FIGS. 36A and 36B illustrates that irradiation of the corticosteroid drug in XRDDS matrix can be used to adjust release rate, particularly for initial burst phase of release and the early period of subsequent steady-state release, with higher drug release from implants irradiated at higher doses. FIG. 36A shows the release rate over time of one example of an extended-release corticosteroid formulation (fluocinolone acetonide) that has not been irradiated (“non-irradiated”) compared to the same formulation that has been irradiated (“irradiated 40 kGy”). FIG. 36B shows a release rate over time of yet another example of an extended-release corticosteroid formulation (fluocinolone acetonide) that has not been irradiated (“non-irradiated”) compared to the same formulation that has been irradiated (“irradiated 40 kGy”). In both cases (FIGS. 36A and 36B), irradiated implants show a higher release rate during initial burst in the first month as well as a higher release rate in the first month of the maintenance phase, as compared to nonirradiated implants.

FIGS. 37A and 37B illustrates adjusting the duration of drug release by controlling the length of the implant, e.g., using a longer or shorter bioerodible or non-bioerodible tubes with shorter tubes having relatively shorter duration of release and longer tubes result in longer duration of drug release. FIG. 37A illustrates exemplary dimensions of a bioerodible tube. FIG. 37B is a graph comparing the duration of release in vivo of an extended-release corticosteroid formulation over time (days) for a 6 mm long implant vs. a 4 mm long implant.

### DETAILED DESCRIPTION

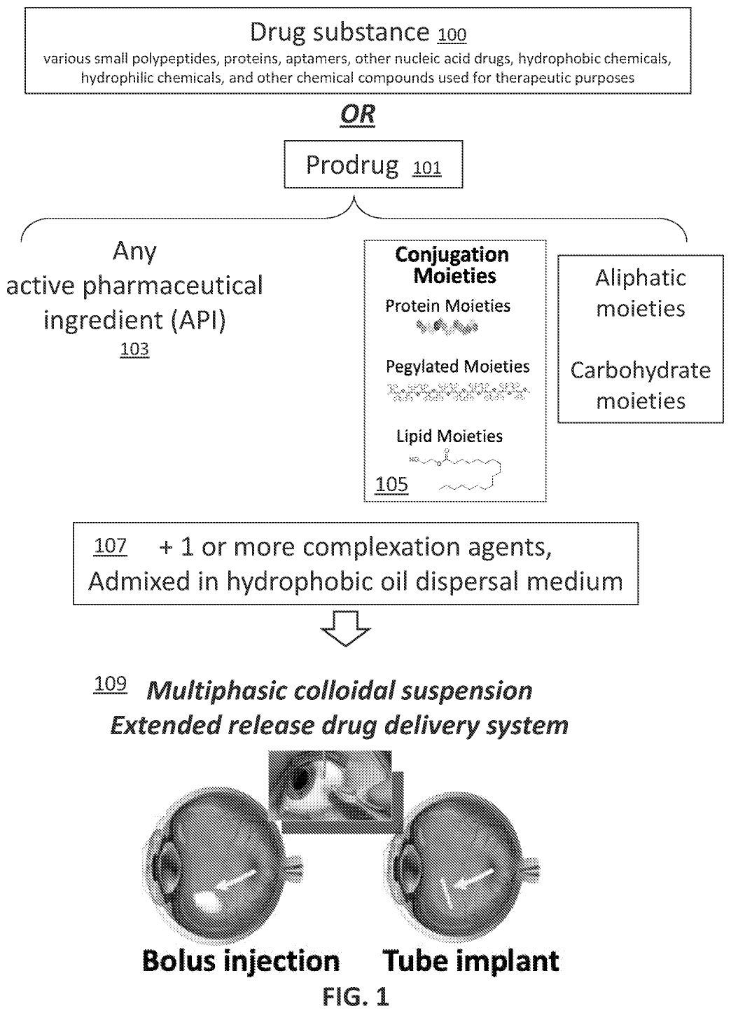

Described herein are compositions of matter and methods of use, for a novel, versatile extended release drug delivery system (XRDDS), for the delivery of various drug substances, in and around the eye, comprising: a drug substance, noncovalently interacting with one or more complexation agent particulates to form drug substance-complex particulates, admixed within a hydrophobic dispersal medium, that collectively forms a stable multiphasic colloidal suspension (FIG. 1).

Herein, drug substance may include 1) various small polypeptides, proteins, aptamers, other nucleic acid drugs, hydrophobic chemicals, hydrophilic chemicals, and other chemical compounds used for therapeutic purposes, that are capable of directly forming noncovalent complexes to one of six classes of complexation agents: fatty acid, organic compounds that can form keto-enol tautomers, charged phospholipid, charged protein, ribonucleic acid, and polysaccharide; and 2) a prodrug of any active pharmaceutical ingredient (API) linked via cleavable covalent bond to a conjugation moiety, wherein the conjugation moiety forms complexes with one of six classes of complexation agents: fatty acid, organic compounds that can form keto-enol tautomers, charged phospholipid, charged protein, ribonucleic acid, and polysaccharide (FIG. 1).

A conjugation moiety is any chemical substance that can be covalently bound to an API. Certain conjugation moieties can be chosen for their ability to provide properties that the native API does not demonstrate, especially the ability to form reversible noncovalent complexes with complexation agents.

A complex is defined as a noncovalent interaction between the drug substance and a complexation agent.

A complexation agent is defined as: a chemical substance formulated as an irregularly shaped particulate ranging in size from 1 nanometer (nm) to 1000 micrometers (m); demonstrates a measurable binding capacity of selected drug substance, defined as a quantity of drug substance bound to a known quantity of complexation agent; demonstrates reversibility of drug binding, defined as a measurable unbound-bound ratio, or Kd, within a specific dispersal medium; and is a chemical substance not previously known or expected to form complexes with the selected drug substance. Binding of drug substance to a complexation agent, either directly or in a prodrug via the conjugation moiety, results in formation of drug substance-complex particulate. Certain well known chemical substances, including additives and excipients utilized in pharmaceutical industry, when formulated as irregular particulates, demonstrate a previously unknown and unexpected property to serve as complexation agents for various drug substances. These include six classes of chemical substances, that, when formulated as irregularly shaped particulates, are not previously known to serve as complexation agents for various drug substances: fatty acid, organic compounds that can form keto-enol tautomers, charged phospholipid, charged protein, ribonucleic acid, and polysaccharide.

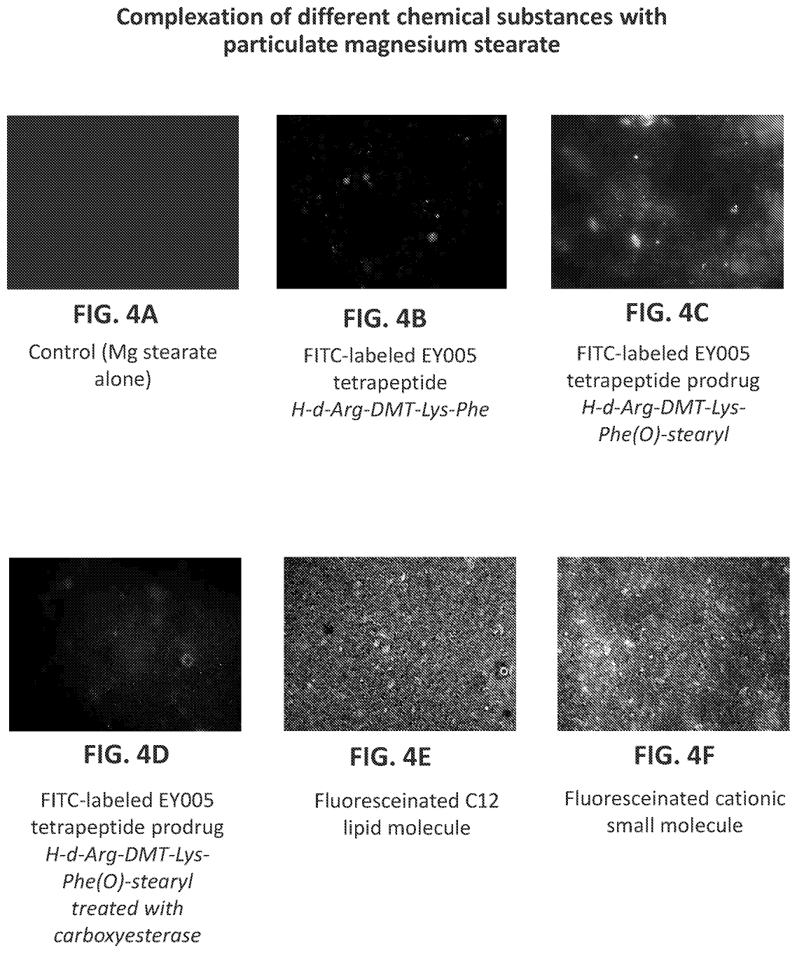

Irregular particulate formulations, not dissolved individual molecules, of magnesium stearate, lecithin, albumin, cyclodextrin, and others all meet the definition for particulate complexation agent for drug substance (FIGS. 4-7), a property not previously known or expected.

A dispersal medium is a vehicle utilized in colloid mixtures. Herein, a dispersal medium is defined as a hydrophobic, viscous oil, selected from among the four classes saturated fatty acid methyl esters, unsaturated fatty acid methyl esters, saturated fatty acid ethyl esters, or unsaturated fatty acid ethyl esters, that when admixed with drug substance-complex particulates, can form the drug substance multiphasic colloidal suspension, and is not previously known to form a multiphasic colloidal suspension with selected drug substance and the chosen complexation agents.

Herein, colloidal suspension is a formulation that is viscous, flowable injectable liquid that forms a stable dispersal of particulates without migration or settling of the particulates (i.e., a colloid mixture).

Multiphasic colloidal suspension containing refers to a colloidal suspension in which the drug substance is present in at least two phases: free, unbound drug substance and drug substance bound to complexation agents (as well as less importantly, drug-drug aggregates). The drug substance-complex particulate serves a reservoir for drug substance when the particulate is admixed into the dispersal medium.

Thus, a drug substance multiphasic colloidal suspension as described herein may be a viscous, flowable injectable liquid that results in stably dispersed drug substance-complex particulates without migration or settling, and may enable free drug substance to dissociate from the drug substance-complex particulates to create a free drug substance concentration in the dispersal medium. The drug substance can freely diffuse through the multiphasic colloidal suspension system to exit the implant into the adjacent ocular physiologic environment. When the drug substance is a prodrug, on exposure of the prodrug to the ocular physiologic environment, the covalent bond linking the conjugation moiety is cleaved, releasing free API.

Formation of the stably dispersed drug substance-complex particulates in the drug substance multiphasic colloidal suspension occurs by admixture, which as defined herein, refers to the mixing and incorporation of drug substance and one or more complexation agents into the dispersal medium by the use of strategies that incorporate a variety of mixing technologies comprising: stand paddle mixing, centrifugal shear mixing, high-shear mixing, ribbon blender, anchor mixers, static mixers, V blenders, planetary mixers, kneading, kneading and folding, whisking, resonant acoustic mixer, Banbury mixer, dispersion mixer, vacuum mixer, high shear rotor mixer, and various other types of mixing technologies. The final admixture may be homogeneously mixed (e.g., having uniform or substantially uniform distribution). In some examples the final admixture may be non-homogeneously mixed (e.g., may have a distribution or gradient of drug-substance complex particulates within the dispersal medium, for example).

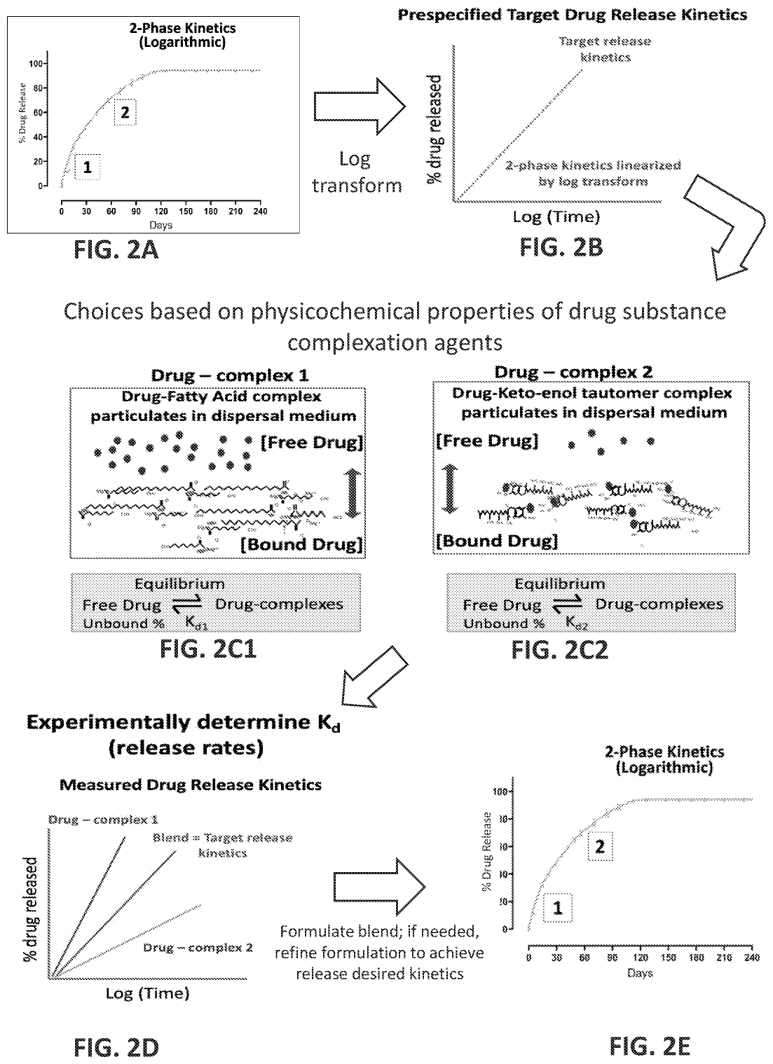

The drug substance multiphasic colloidal suspension enables a drug delivery system because the particulates are a reservoir of bound drug substance, each with a unique binding capacity and Kd (unbound-bound ratio), which in turn determines the composite amount of free drug substance in the dispersal medium. Knowledge of the Kd and the binding capacity of each drug substance-complex particulate can be used to calculate the total amount of free drug substance in the system, which in turn determines the rate and amount of release. The relative ratio and amounts of different drug substance-complex particulates can be adjusted in a manner to create a calculatable unbound free drug substance within the system (FIGS. 2A-2E). The dynamic change of unbound, free drug substance within the system over the life of the implant is determined by the binding capacity and Kd of the drug substance-complex particulates within the drug substance multiphasic colloidal suspension.

In the methods and compositions described herein, the drug substance multiphasic colloidal suspension is injectable through a 20-gauge through 30-gauge size needle (depending on utilization) and provides stable dispersion of particulates without migration or settling when exposed to an ocular physiologic environment for the duration of the implant's lifetime (1 to 12 months). An ocular physiologic environment is defined as in vitro conditions with phosphate buffered saline (or comparable aqueous solvent) at 37° C. containing enzymes and proteins normally found in vitreous (representing injection into the vitreous) or with phosphate buffered saline at 37° C. containing plasma (representing injection into various periocular tissues). Alternatively, ocular physiologic environment may represent injection of the implant in vivo into the vitreous or into periocular tissues.

The drug substance multiphasic colloidal suspension also manifests the property of biodegradability when exposed to an ocular physiologic environment wherein biodegradability occurs by dissolution of the dispersal medium. The rate of biodegradation is proportional to the degree of solubility of the dispersal medium in the ocular physiologic environment. A dispersal medium with higher solubility will enable faster biodegradation of the multiphasic colloidal suspension when exposed to an ocular physiologic environment, while a dispersal medium with lower solubility will enable slower biodegradation of the multiphasic colloidal suspension when exposed to an ocular physiologic environment. This property of the drug substance multiphasic colloidal suspension can be used along with the volume of injected implant to determine durability of the implant in an ocular physiologic environment.

Formulation of the drug substance in the multiphasic colloidal suspension, termed the implant, can be administered in and around the eye, i.e., into the vitreous humor, into the aqueous humor, into the suprachoroidal space, under the retina, under the conjunctiva, beneath Tenon's capsule, into orbital tissue, to produce sustained release of therapeutic levels of drug substance within ocular tissues for desired duration (1 to 12 months), for the treatment of various diseases and disorders.

The multiphasic colloidal suspension extended release compositions described herein (e.g., extended release drug delivery system, XRDDS) may include drug substance admixed with one or more particulate complexation agents to form “drug-complex” particulates, which are combined and dispersed within a selected dispersal medium to form a stable multiphasic colloidal suspension.

Colloids are mixtures in which particulate substances are stably dispersed within a vehicle, called a dispersal medium, but do not settle or migrate. This differentiates a colloid from a suspension in which the particles settle within the suspension vehicle due to gravity. Typical particulate size for colloids is in the nanometer range. In colloids, the defining characteristic of the mixture is that particulates remain stably dispersed with minimal settling or migration. Colloid mixture in which particulates are dispersed in a liquid is called a “sol.” Colloid mixtures in which particulates are dispersed in a solid or semisolid is called a “solid colloid.” Colloid mixtures in which particulates are stably dispersed in a viscous semi-solid or solid dispersal medium have not been given a defined named. Herein, we refer to stably dispersed particulates as “colloidal suspension.” In methods and compositions described herein, the dispersal medium may be a hydrophobic dispersal medium that facilitates a stable colloidal suspension. A drug substance multiphasic colloidal suspension is a suspension in which the drug substance is present in more than one phase, including free drug, drug-drug aggregates, and most importantly, drug noncovalently bound to complexation agent particulates.

Complexation occurs in two physicochemical circumstances. In one case, complexation occurs with noncovalent interactions between individual molecules (e.g., receptor-ligand interactions). This type of complexation is termed molecular complexation and is not contemplated in the current composition.

The second circumstance involves a molecule of a chemical substance, in this case, molecule of drug, that noncovalently binds or adsorbs to a surface of a particulate, in this case, a complexation agent. This type of complexation is termed particulate complexation. Different particulate adsorbents, or complexation agents, have different sorptive properties based on size and shape of particulate, functional groups present at the surface, and the surface irregularity and porosity of the particulate. The utility of particulate complexation has been recognized in other disciplines, including soil sciences, wherein a chemical adsorbent (e.g., alumina, silica gel, activated charcoal) interacts with specific chemicals (frequently contaminants) in soil; the hydrocarbon industry, wherein adsorbents (e.g., polypropylene, vermiculite, perlite, polyethylene, others) are used to clean oil spills or to remove residual oil from drilling and fracking equipment; and industrial coatings (e.g., zeolite, silica gel, aluminum phosphate), wherein adsorbents are used to bind chemical substances for various purposes (i.e., lubrication, surface cooling).

In medical applications, adsorbents are used for the treatment of acute poisoning by ingestion (e.g., activated charcoal, calcium polystyrene sulfate, aluminum silicate) where the adsorbent binds the toxin to limit adsorption from the gut into systemic circulation. In the pharmaceutical industry, principles of adsorption complexation are used to understand chemistry of drug binding to plasma proteins in the blood, drug coatings on solid scaffolds for in situ drug release (e.g., drug-eluting stents), and affixing excipients to insoluble drugs in order to improve oral bioavailability and gut absorption.

The methods and compositions described herein may utilize particulate complexation, wherein complexation agents thus are chemicals compatible with ocular tissues that, when formulated as an irregularly shaped particulates, have the capacity of noncovalently binding drug substance, forming drug substance-complex particulates. One or more drug substance-complex particulates are incorporated and admixed into a hydrophobic dispersal medium to form a stable multiphasic colloidal suspension, that is safely delivered into and around the eye, to produce continuous exposure to predictable therapeutic levels of drug substance in ocular tissues for a desired duration of treatment. Complexation agents are selected from one of six classes of chemical substances, including fatty acid, organic compounds that can form keto-enol tautomer, charged phospholipid, charged protein, nucleic acid, and polysaccharides.

When the drug substance is a prodrug, the conjugation moiety of the prodrug is specifically chosen for its ability to complex, or form noncovalent interactions, with one or more particulate complexation agents to form prodrug-complex particulates. One or more prodrug substance-complex particulates are incorporated and admixed into a hydrophobic dispersal medium to form a stable multiphasic colloidal suspension, that is safely delivered into and around the eye, to produce continuous exposure to predictable therapeutic levels of drug substance in ocular tissues for a desired duration of treatment. Complexation agents are selected from one of six classes of chemical substances, including fatty acid, organic compounds that can form keto-enol tautomer, charged phospholipid, charged protein, nucleic acid, and polysaccharides.

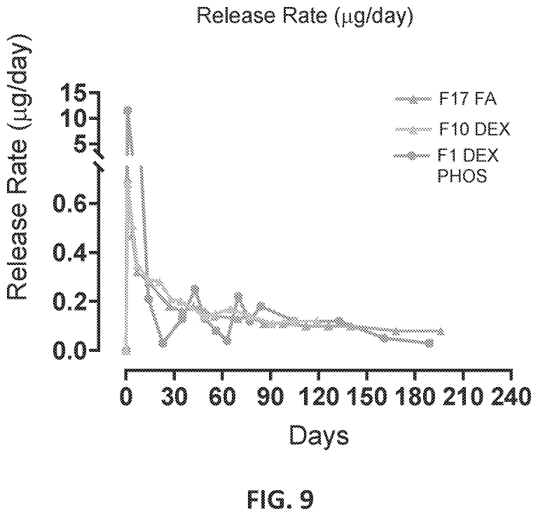

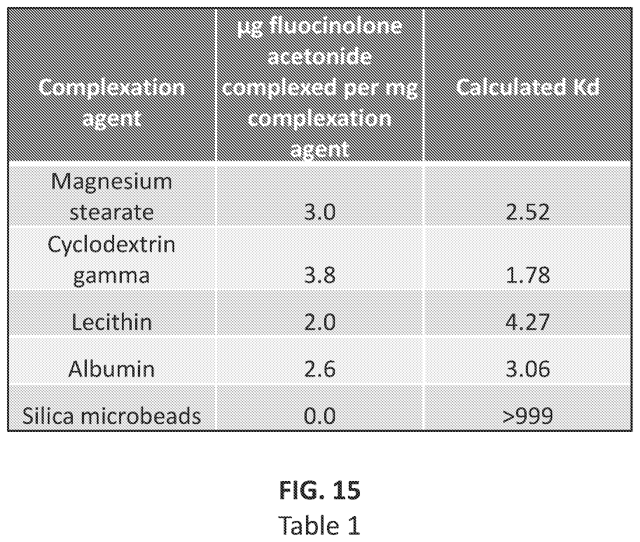

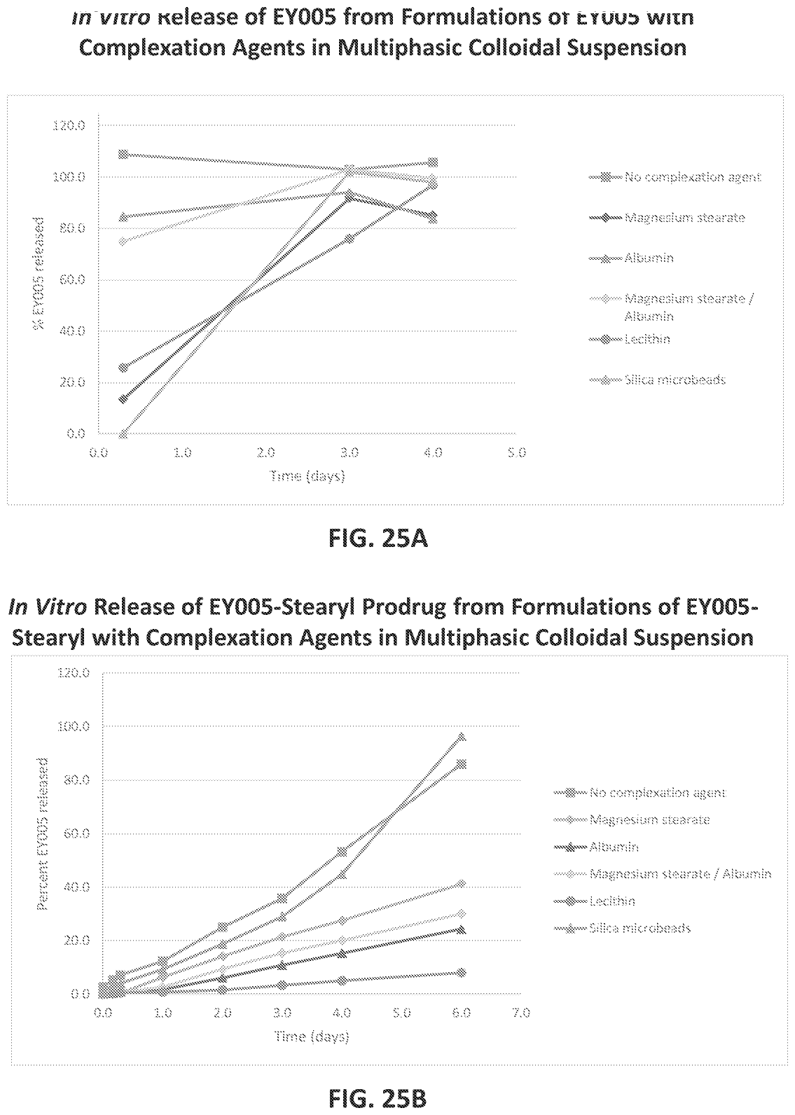

The methods and compositions described herein disclose a new property, not previously recognized, of these six classes of chemical substances, fatty acid, organic compounds that can form keto-enol tautomer, charged phospholipid, charged protein, nucleic acid, and polysaccharides, that, when in the form of an irregularly shaped particulate with irregular surface, can serve as an effective complexation agent for drug substances. The criteria for complexation agent includes the following four features: (1) drug substance binds to the particulate complexation agent and this is demonstrable by microscopy imaging (FIGS. 4A-4F, 5A-5F, 6A-6F and 7A-7F); (2) when particulate of substance is added to a solution of drug substance, upon centrifugation and pulldown of the particulates, pharmacologically significant quantities of drug substance are observed to be complexed to the particulates, providing a quantitative metric of binding capacity of the complexation agent (see FIGS. 15, 18, 20, 24); (3) drug substance-complex particulates when resuspended in appropriate dispersal medium, demonstrate partial release of drug, allowing determination of Kd or unbound-bound fraction of drug for a given drug substance-complexation agent pair in a particular dispersal medium (see FIGS. 25A-25B); and (4) the drug substance-complex particulate provide a useful pharmacokinetic release profile when admixed into the dispersal medium to form the drug substance multiphasic colloidal suspension (see FIG. 9). Collectively, these four properties define a complexation agent and enable the presently described complexation-based XRDDS.

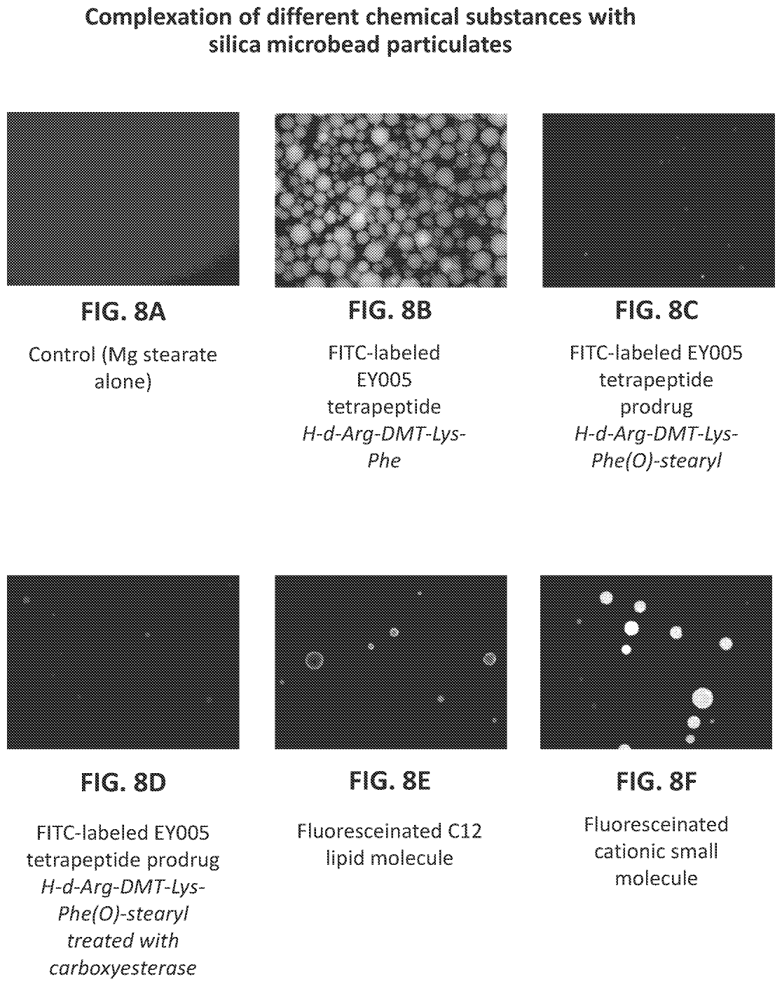

In contrast, spherical particulates with a spherical smooth surface and non-reactive coating, including for example silicone beads, latex beads, and certain polymeric particulates, fail to form complexes with drug substance (FIGS. 8A-8F), and therefore are excluded from the methods and compositions described herein.

One class of complexation agents is fatty acid, which is a carboxylic acid with an aliphatic chain, which may be either saturated or unsaturated, and may be in the form of a salt or ester. For example, the fatty acid may have a chemical formula of CH3(CH2)ₙCOOH where n is equal to between 4 and 30. The fatty acid may comprise one of: Tetradecanoic acid, pentadecanoic acid, (9Z)-hexadecenoic acid, Hexadecanoic acid, Heptadecanoic acid, Octadecanoic acid, (9Z,12Z)-octadeca-9,12-dienoic acid, (9Z,12Z,15Z)-octadeca-9,12,15-trienoic acid, (6Z,9Z,12Z)-octadeca-6,9,12-trienoic acid, (5E,9E,12E)-octadeca-5,9,12-trienoic acid, (6Z,9Z,12Z,15Z)-octadeca-6,9,12,15-tetraenoic acid, (Z)-octadec-9-enoic acid, (11E)-octadec-11-enoic acid, (E)-octadec-9-enoic acid, nonadecanoic acid, eicosanoic acid etc.). The fatty acid may be an unbranched fatty acid between C14 and C20. The fatty acid may be a saturated fatty acid comprising one of: myristic acid (tetradecanoic acid), palmitic acid (hexadecanoic acid), stearic acid (octadecanoic acid), arachidic acid (eicosanoic acid). Specific examples of salt form fatty acids include magnesium stearate (FIGS. 4A-4F), magnesium palmitate, calcium stearate, calcium palmitate, and others.

One class of complexation agents is organic compounds that can form keto-enol tautomers. Tautomers refer to molecules capable of undergoing chemical equilibrium between a keto form (a ketone or an aldehyde) and an enol form (an alcohol). Usually, a compound capable of undergoing keto-enol tautomerization contains a carbonyl group (C═O) in equilibrium with an enol tautomer, which contains a pair of doubly bonded carbon atoms adjacent to a hydroxyl (—OH) group, C═C—OH as depicted herein:

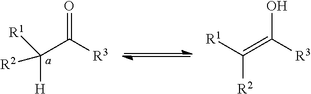

The relative concentration of the keto and enol forms is determined by the chemical properties of the specific molecule and the chemical microenvironment, including equilibrium, temperature or redox state. Organic compounds capable of keto-enol tautomerization include but are not limited to phenols, tocopherols, quinones, ribonucleic acids, and others.

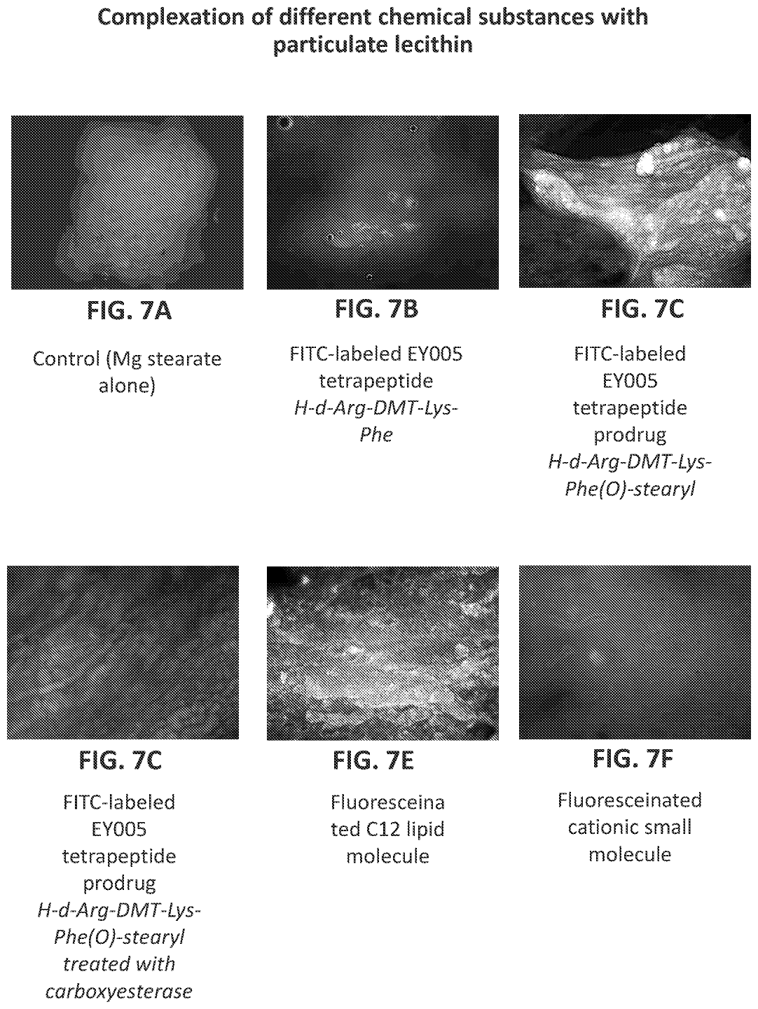

One class of complexation agents is charged phospholipid. In general, phospholipids consist of a glycerol molecule, two fatty acids, and a phosphate group that is modified by an alcohol, wherein the polar head of the phospholipid is typically negatively charged. Examples include lecithin (FIGS. 7A-7F), phosphatidylcholine, phosphatidylethanolamine, phosphatidylserine, different phospholipids in oil, and many others, which may be used individually or in combination to serve as complexation agents. Anionic phospholipids may comprise one of: phosphatidic acid, phophatidyl serine, sphingomyelin or phophatidyl inositol. In some instances, synthetic, ionizable phospholipids with positive charge can manufactured, including but not limited to examples such as DLin-MC3-DMA. Additional cationic phospholipids may comprise one of: cationic triesters of phosphatidylcholine; 1,2-dimyristoylsn-glycerol-3-phosphocholine (DMPC); 1,2-dioleoyl-sn-glycerol-3-phosphocholine (DOPC); 1,2-bis(oleoyloxy)-3-(trimethylammonio)propane (DOTAP); 1,2-dioleoyl-sn-glycerol-3-phosphoethanolamine (DOPE); 1,2-dipalmitoyl-sn-glycerol-3-phosphocholine (DPPC); 1,2-dioleoyl-sn-glycerol-3-ethylphosphocholine (EDOPC); 1,2-dimyristoyl-sn-glycerol-3-ethylphosphocholine (EDMPC); 1,2-dipalmitoyl-sn-glycerol-3-ethylphosphocholine (EDPPC). In pharmaceutical sciences, phospholipids have been used for drug formulation and delivery applications to improve bio-availability, reduced toxicity, and improved cellular permeability. However, in the methods and compositions described herein, phospholipids are used as a complexation agent particulate to noncovalently bind the drug substance and form drug substance-complex particulates for the purpose of regulating free drug substance in the dispersal medium of the stable multiphasic colloidal suspension in which the drug substance-complex particulates are incorporated and dispersed therein.

In some examples, an anionic phospholipid may form noncovalent complexation with a cationic conjugation moiety of a prodrug. A cationic phospholipid may form noncovalent complexation with an anionic conjugation moiety of a prodrug.

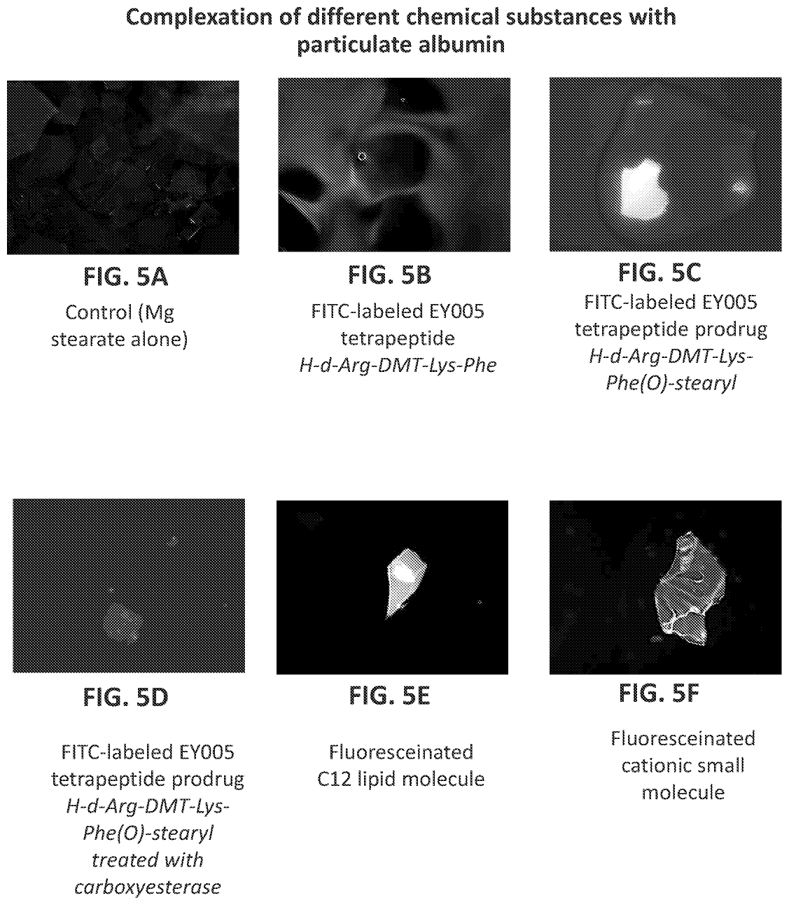

One class of complexation agents is charged protein. Proteins are large biomolecules and macromolecules that comprise one or more long changes of amino acid residues. Amino acids that make up proteins may be positive, negative, neutral, or polar in nature, and collectively, the amino acids that comprise the protein give it its overall charge. A variety of proteins, based on size, molecular weight, ability to readily form particulates, and compatibility with ocular tissues could serve as complexation agents. The charge of the protein will determine its compatibility with a specific drug substance such that negatively charged proteins will readily complex with positively charged drug substance, while positively charged proteins (e.g., Arg-Gln-Ile-Arg-Arg-Ile-Ile-Gln-Arg-NH₂ and synthetic peptides with positive charge) will readily complex with negatively charged drug substance. Examples of proteins that could serve as complexation agents include albumin (FIGS. 5A-5F) and collagen.

One class of complexation agents is nucleic acids, biopolymer macromolecules comprised of nucleotides, comprised of a 5-carbon sugar, a phosphate group, and a nitrogenous base. The importance of nucleic acids for biologic function and encoding genetic information is well established. However, nucleic acids also have a variety of applications, including nucleic acid enzymes (e.g., carbon nanomaterials), aptamers (e.g., for formation of nucleic acid nanostructures and therapeutic molecules that function in an antibody-like fashion), and aptazymes (e.g., which can be used for in vivo imaging). In pharmaceutical sciences, specially engineered nucleic acids have been considered and applied for use in carrier-based systems in which the nucleic acid serves as a carrier system for various types of drugs. However, in the methods and compositions described herein, nucleic acids are considered not as a carrier system but rather as a complexation agent, as they are highly negatively charged and thus, formulated as a particulate, could then serve as a complexation agent for positively charged drug substance.

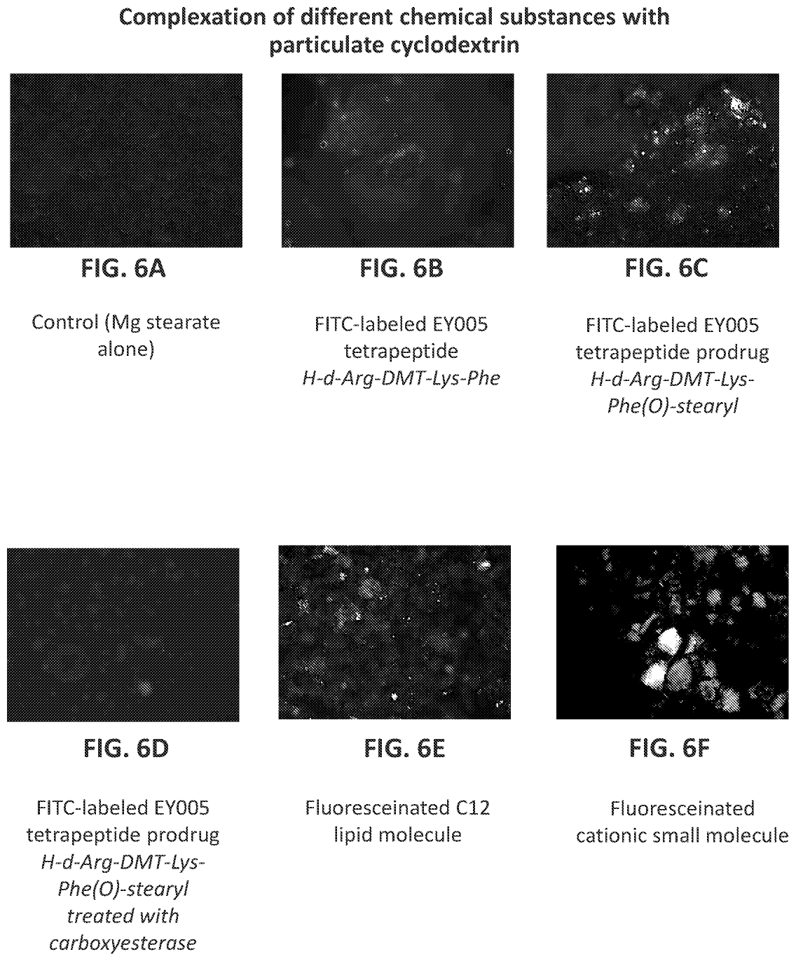

One class of complexation agent is polysaccharides, long chain polymeric carbohydrates composed of monosaccharide units bound together by glycosidic linkages. Frequently, these are quite heterogenous, containing slight modifications of the repeating monosaccharide unit. Depending on structure, they can be insoluble in water. Complexation of polysaccharide particulate complexation agents to drug substances can occur through various electrostatic interactions and is influenced by charge density of drug substance and polysaccharide, ratio of polysaccharide complexation agent drug substance, ionic strength, and other properties. Examples of polysaccharides that could serve as complexation agents include a ringed polysaccharide molecule, cyclodextrins (FIGS. 6A-6F), a clathrate, cellulose, pectins, or acidic polysaccharides, polysaccharides that contain carboxyl groups, phosphate groups, or other similarly charged groups.

The complexation agent may be a compound containing metal ions.

In any of these therapeutic compositions an ionic coordination complexation may occur around a central ion forming extensive noncovalent interactions. The central ion may be a central metal ion comprising one of: copper, iron, zinc, platinum, or lithium.

Ionic coordination complexation is a chemical complexation process around a central ion, usually a metal, capable of forming extensive noncovalent electrostatic interactions with a wide range of chemical substances. This is one of the most common chemical processes in nature. The avidity of binding is variable amongst different coordination ions, some of which may be nearly irreversible while others manifest relatively labile binding. Central metal ions include copper, iron, zinc, platinum, lithium, others. Three classes that can serve as a complexation agent for drug delivery are chelators (EDTA), complexation to certain specific metals (platinum, lithium, lanthanum) and molecules with metalloprotein elements (hemoglobin, porphyrin, superoxide dismutase, and others with zinc or copper binding domains).

The complexation agent may comprise a chelator configured for complexation to a metal, a metalloprotein, or a superoxide dismutase (SOD). The complexation agent may comprise a chelator configured for complexation to one or more of: platinum, lithium, lanthanum, hemoglobin, porphyrin, zinc binding domains, or superoxide dismutase (SOD).

In the methods and compositions described herein, a selected drug substance has specific avidity for, and complexes with, a given complexation agent, forming a drug substance-complex particulate. This avidity can be measured as Kd, the unbound-bound fraction of a drug substance for a given drug substance-complex particulate in a selected dispersal medium.

Another property of drug substance-complex particulate is the binding capacity, defined as a quantity of drug substance bound to a known quantity of complexation agent.

The avidity and binding capacity of the drug substance for a particular complexation agent (FIGS. 15, 18, 20, 24) thus serves to limit the free drug available for release from the drug substance-complex particulate for a given dispersal medium.

Thus, in the multiphasic colloidal suspension comprised of one or more drug substance-complex particulates incorporated into a hydrophobic dispersal medium, rather than use of complexation to improve bioavailability, formulations of the multiphasic colloidal suspension use complexation to limit free, unbound drug substance available for release from a given dispersal medium of the multiphasic colloidal suspension.

Drug substance formulated in the present extended release drug delivery system (XRDDS), the multiphasic colloidal suspension, may include various small polypeptides, proteins, aptamers, other nucleic acid drugs, hydrophobic chemicals, hydrophilic chemicals, and other chemical compounds used for therapeutic purposes that are capable of directly forming noncovalent complexes to one of six classes of complexation agents: fatty acid, organic compounds that can form keto-enol tautomers, charged phospholipid, charged protein, ribonucleic acid, and polysaccharide.

The drug substance directly forms noncovalent avid interactions (or binding) to one of six different classes of substances formulated as irregularly shaped particulates: fatty acid, organic molecules that can form keto-enol tautomers, charged phospholipid, charged protein, nucleic acid, and polysaccharides. The resultant drug substance-complex particulates admixed into dispersal medium regulates the release of free, unbound drug within the multiphasic colloidal suspension, enabling controlled, extended release from the formulated implant upon administration into ocular physiologic environment.

The drug substance formulated in the multiphasic colloidal suspension may also be a prodrug of any active pharmaceutical ingredient (API) linked via cleavable covalent bond to a conjugation moiety, wherein the conjugation moiety forms complexes with one of six classes of complexation agents: fatty acid, organic compounds that can form keto-enol tautomers, charged phospholipid, charged protein, ribonucleic acid, and polysaccharide.

The prodrug has formula (I):

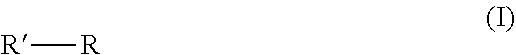

where R′ is any active pharmaceutical ingredient (API) that is covalently linked via cleavable bond to R, a conjugation moiety that forms noncovalent complexes with one of five classes of complexation agents, and the covalent bond linking R′ and R may be removed by enzymatic cleavage, catalysis, hydrolysis, or other reaction to yield free API R′ and conjugation moiety R, where R is selected from: a C4-C30 lipid moiety (fatty acid or fatty alcohol), an C4-C30 straight-chain or branched aliphatic moiety, a 2-mer to 30-mer peptide moiety, a pegylated moiety, or a carbohydrate moiety.

The prodrug may be a product of a condensation or esterification reaction between API and conjugation moiety.

In pharmacology, prodrugs are chemical modifications of the API. Prodrugs are metabolized within the host either by tissue enzymes or by hydrolysis into the free API and the inactive conjugation moiety. Prodrugs are generally used to modify the API's physicochemical properties to improve absorption, bioavailability, or pharmacokinetics (PK). However, in the methods and compositions described herein, the purpose of the prodrug strategy is to optimize the drug's physicochemical properties for compatibility with the multiphasic colloidal suspension extended release drug delivery system (XRDDS). In most cases, this provides the API with a regulated release rate than cannot otherwise be achieved with non-prodrug native form of the API.

The covalently linked conjugation moieties of drug substances form noncovalent avid interactions, binding to one of six different classes of substances formulated as irregularly shaped particulates: fatty acid, organic molecules that can form keto-enol tautomers, charged phospholipid, charged protein, nucleic acid, and polysaccharides. The formation of prodrug-complex particulates optimizes the API's physicochemical properties for compatibility with the multiphasic colloidal suspension, wherein the prodrug-complex particulates admixed into dispersal medium regulates the release of free, unbound prodrug within the multiphasic colloidal suspension, enabling controlled, extended release from the formulated implant upon administration into ocular physiologic environment.

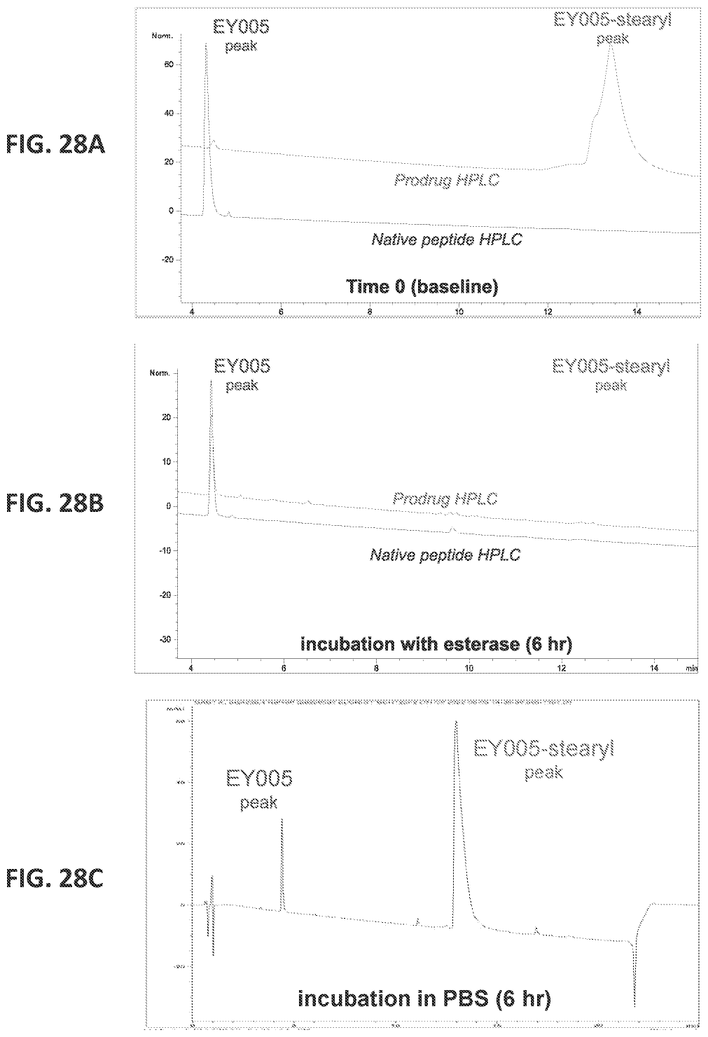

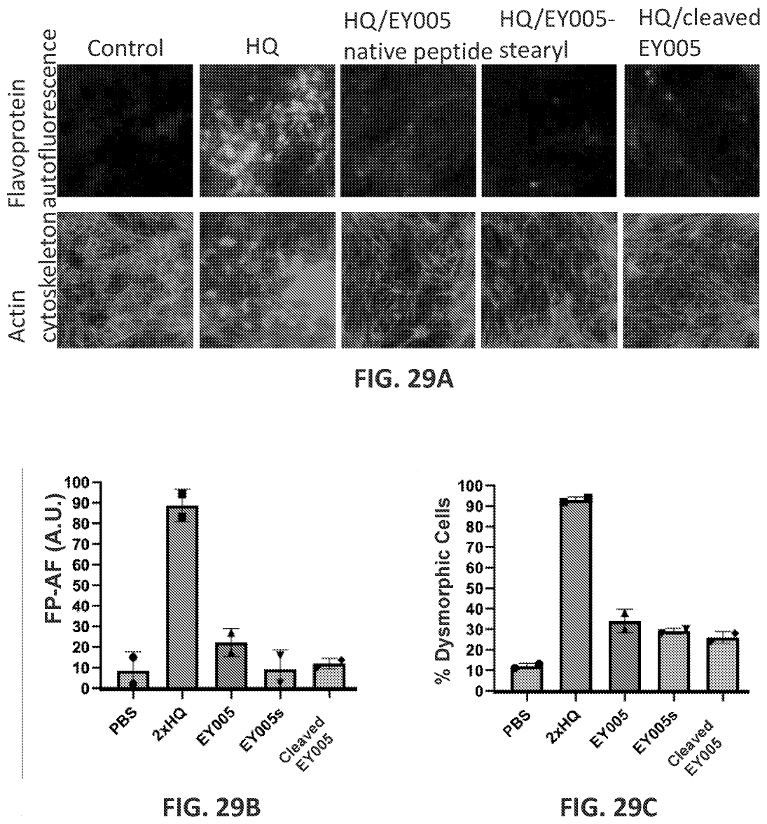

When the drug substance is a prodrug, a key feature of the prodrug is that the bond linking API to the conjugation moiety is readily cleaved by enzymatic reaction, catalysis, hydrolysis, or other chemical reaction (FIGS. 28A-28C). Upon cleavage of this bond in the prodrug, the released API retains full bioactivity for its mechanism of action (FIGS. 29A-29C).

Numerous metabolizing enzymes have been detected in ocular tissues, including esterases, peptidases, phosphatases, oxime hydrolases, ketone reductases, and others. The linkage to the conjugation moiety for prodrug described herein may be configured to achieve specific cleavage by any of these metabolizing enzymes.

The cleavable covalent bond may comprise one of: an ester bond, a hydrazone bond, an imine bond, a disulfide bond, a thioester bond, a thioether bond, a phosphate ester bond, a phosphonate ester bond, a boronate ester bond, an amide bond, a carbamate ester bond, a carboxylate ester bond, carbonate ester bond, or others known to those practiced in the art of medicinal chemistry.

Ester prodrugs in particular may be desirable since the ocular tissues contain abundant esterase activity.

In some examples of prodrugs, cleavage and release of the free API can be assessed in an in vitro release assay, wherein the prodrug is incubated in a solution containing carboxyesterase (or other natural or synthetic esterase), isolated vitreous recovered from animal (e.g., pig, rabbit, etc.), or isolated vitreous recovered from human donor, at 370 Celsius, 250 Celsius, or other temperatures. Analytic methods such as HPLC or mass spectrometry can be used to calculate the amount of free API and intact prodrug, at various timepoints after start of incubation (FIG. 28B).

In some examples, cleavage and release of the free API can be assessed in an in vitro release assay, wherein the prodrug is incubated in media, at 370 Celsius, 250 Celsius, or other temperatures. Analytic methods such as HPLC or mass spectrometry can be used to calculate the amount of free API and intact prodrug, at various timepoints after start of incubation (FIG. 28C).

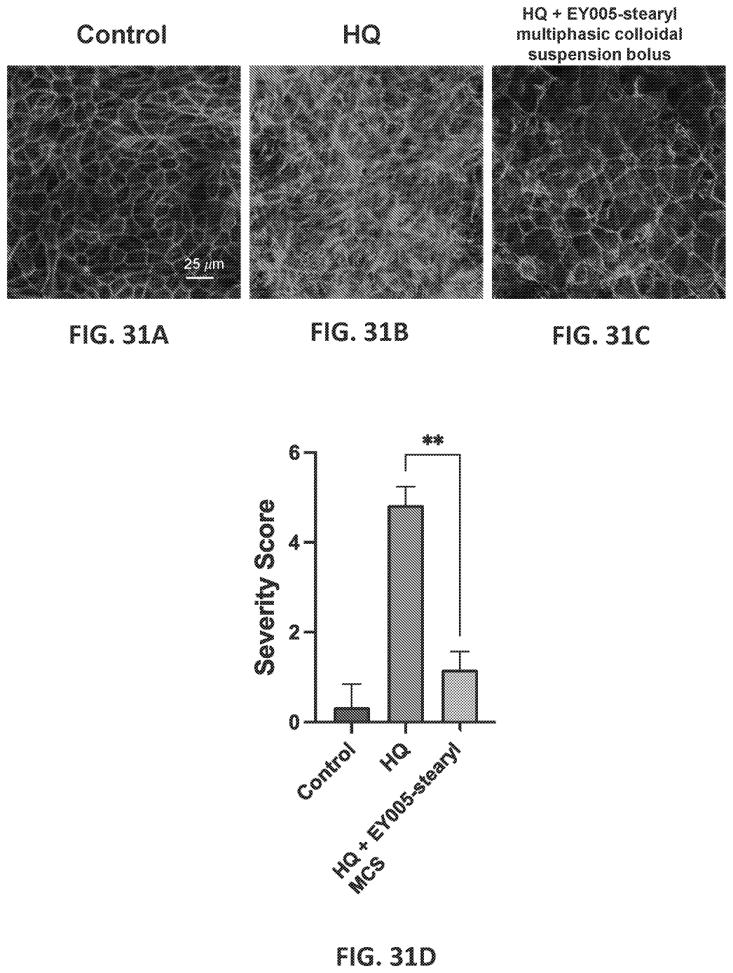

In some examples, cleavage and release of the free API can be assessed following in vivo injection of the prodrug into the vitreous cavity or periocular tissues of a preclinical animal model (e.g., mouse, rat, rabbit, pig, etc.), wherein ocular tissue is recovered, and analytic methods such as HPLC or mass spectrometry can be used to calculate the amount of free API and intact prodrug, at various timepoints after in vivo injection (FIGS. 31A-31D).

In general, the conjugation moiety, R, to which the API is covalently linked, is not selected on the basis of bioactivity for a target or mechanism of action.

Although not a preferred embodiment, disclosed herein are drug substances comprised of homo- or hetero-dimers, trimers, multimers of any drug substance, either linked together directly or indirectly to a chemical substance that serves a linker moiety, which could functionally serve as a cleavable conjugation moiety.

As described herein, the API, R′, may be covalently linked to conjugation moiety R, selected from among one of the following five classes of chemical substances: a C4-C30 lipid moiety, a C4-C30 straight-chain or branched aliphatic moiety, a 2-mer to 30-mer peptide moiety, a pegylated moiety, or a carbohydrate moiety.

One class of conjugation moieties is C4-C30 lipid moiety, with or without a preceding linker moiety that bonds the lipid moiety to the API. Herein, lipid is defined as organic compounds that are insoluble in water but soluble in organic solvents. Lipids include fatty acids, fatty alcohols, glycerolipids, glycerophospholipids, sphingolipids, saccharolipids, polyketides (derived from condensation of ketoacyl subunits), sterol lipids, prenol lipids (derived from condensation of isoprene subunits), phospholipids, oils, waxes, and steroids.

The fatty alcohol may comprise one or more of: tert-butyl alcohol, tert-amyl alcohol, 3-methyl-3-pentanol, 1-heptanol (enanthic alcohol), 1-octanol (capryl alcohol), 1-nonanol (pelargonic alcohol), 1-decanol (decyl alcohol, capric alcohol), undecyl alcohol (1-undecanol, undecanol, hendecanol), dodecanol (1-dodecanol, lauryl alcohol), tridecyl alcohol (1-tridecanol, tridecanol, isotridecanol), 1-tetradecanol (myristyl alcohol), pentadecyl alcohol (1-pentadecanol, pentadecanol), 1-hexadecanol (cetyl alcohol), cis-9-hexadecen-1-ol (palmitoleyl alcohol), heptadecyl alcohol (1-n-heptadecanol, heptadecanol), 1-octadecanol (stearyl alcohol), 1-octadecenol (oleyl alcohol), 1-nonadecanol (nonadecyl alcohol), 1-eicosanol (arachidyl alcohol), 1-heneicosanol (heneicosyl alcohol), 1-docosanol (behenyl alcohol), cis-13-docosen-1-ol (erucyl alcohol), 1-tetracosanol (lignoceryl alcohol), 1-pentacosanol, 1-hexacosanol (ceryl alcohol), 1-heptacosanol, 1-octacosanol (montanyl alcohol, cluytyl alcohol), 1-nonacosanol, 1-triacontanol (myricyl alcohol, melissyl alcohol).

The fatty acid may comprise one or more of: Tetradecanoic acid, pentadecanoic acid, (9Z)-hexadecenoic acid, Hexadecanoic acid, Heptadecanoic acid, Octadecanoic acid, (9Z,12Z)-octadeca-9,12-dienoic acid, (9Z,12Z,15Z)-octadeca-9,12,15-trienoic acid, (6Z,9Z,12Z)-octadeca-6,9,12-trienoic acid, (5E,9E,12E)-octadeca-5,9,12-trienoic acid, (6Z,9Z,12Z,15Z)-octadeca-6,9,12,15-tetraenoic acid, (Z)-octadec-9-enoic acid, (11E)-octadec-11-enoic acid, (E)-octadec-9-enoic acid, nonadecanoic acid, and eicosanoic acid.

One class of conjugation moieties is C4-C30 straight-chain or branched aliphatic moiety, with or without a preceding linker moiety that bonds the aliphatic hydrocarbon, to the API. This class include alkanes, alkenes, and alkynes, and other hydrocarbon moieties made up of 4 to about 30 carbons and can include unbranched, branched, and cyclic groups.

One class of conjugation moieties is peptide moiety, with or without a preceding linker moiety that bonds the peptide to API, wherein the peptide moiety comprises a natural or synthetic amino acid polymer or polypeptide chain with length of 2-mer to 30 mer, which may be anionic, cationic, or neutral in charge and contain homogeneous or heterogeneous amino acid repeats.

Examples of anionic peptide sequences that may serve as conjugation moiety groups R include but are not limited to: poly-aspartic acid (aspartate), poly-glutamic acid (glutamate), peptides comprised of poly-(aspartic acid-glutamic acid) or poly-(glutamic acid-aspartic acid) repeats.

Examples of cationic peptide sequences that may serve as conjugation moiety groups R include but are not limited to: poly-lysine, poly-arginine, poly-histidine, peptides comprised of poly-(lysine-arginine) (or arginine-lysine) repeats, peptides comprised of poly-(lysine-histidine) (or histidine-lysine) repeats, peptides comprised of poly-(arginine-histidine) (or histidine-arginine) repeats, peptides comprised of poly-(lysine-arginine-histidine) repeats, peptides comprised of poly-(lysine-histidine-arginine) repeats, peptides comprised of poly-(arginine-lysine-histidine) repeats, peptides comprised of poly-(arginine-histidine-lysine) repeats, peptides comprised of poly-(histidine-arginine-lysine) repeats, peptides comprised of poly-(histidine-lysine-arginine) repeats.

The peptide moiety may have one or more PEGylation sites for addition of polyethylene glycol (PEG) groups.

The peptide moiety may have one or more sites for modification by addition of sugar or carbohydrate molecules, including glycosylation.

One class of conjugation moieties is pegylated compound moiety, with or without a preceding linker moiety that bonds the pegylated compound to the API, including polyethylene glycol (PEG) polymers of linear, branched, Y-shaped, or multi-arm geometries, pegylated peptides or proteins, or pegylated succinates such as succinimidyl succinate.

One class of conjugation moieties is carbohydrate molecular moiety, with or without a preceding linker moiety that bonds the carbohydrate to the API, including but not limited to monosaccharides or oligosaccharides of 2 to 20 sugars. The carbohydrate molecule may comprise one or more of: glucose, galactose, lactose, mannose, ribose, fucose, N-acetylgalactosamine, N-acetylglucosamine, N-acetyleneuraminic acid, or an epimer or derivative of any of these.

One example of how a prodrug may be incorporated into a multiphasic colloidal suspension is from among the class of mitochondria-targeted tetrapeptides (MTT), which can be used to form a prodrug that is a product of a condensation or esterification reaction, of formula, (II):

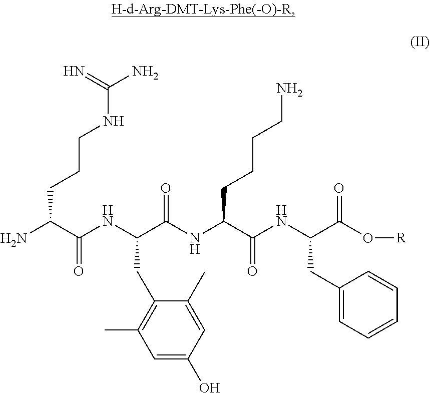

where R is covalently linked via ester bond at the hydroxyl group of the amino acid in the 4th position of the MTT and is selected from among one of the following five classes of chemical substances: a C4-C30 lipid moiety, an C4-C30 straight-chain or branched aliphatic moiety, a 2-mer to 30-mer peptide moiety, a pegylated moiety, or a carbohydrate moiety.

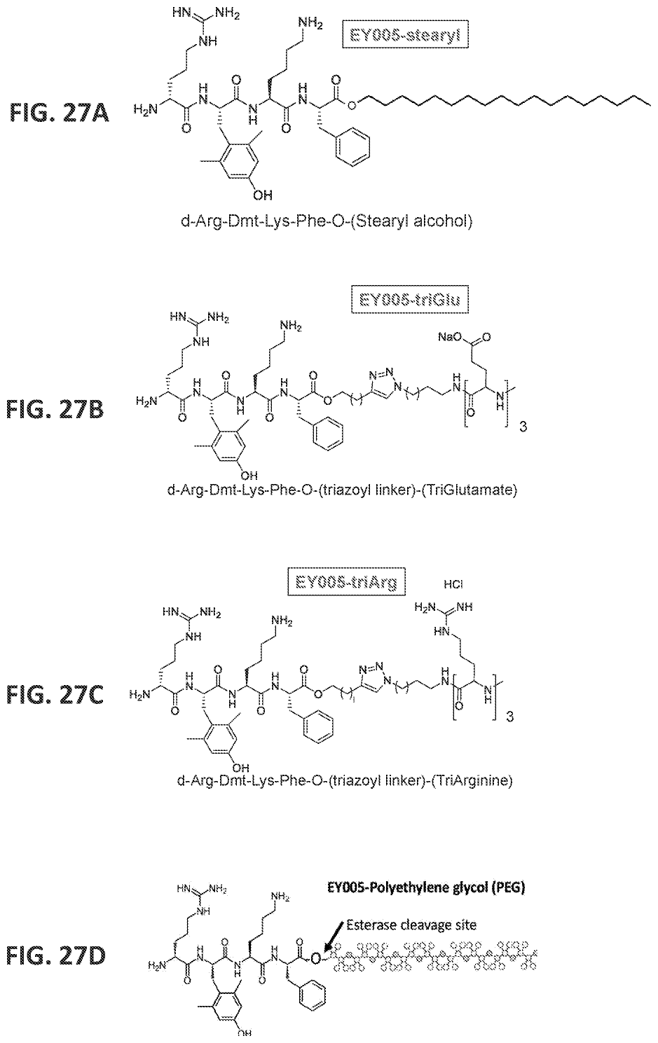

In some examples, the prodrug H-d-Arg-DMT-Lys-Phe(—O)—R has the formula of: H-d-Arg-DMT-Lys-Phe (—O—)-nonpolar lipid. The nonpolar lipid may include one of several molecules, including octadecyl (where the —O—R is derived from stearyl alcohol) (FIG. 27A) or hexadecyl (where the —O—R is derived from palmityl alcohol) or other comparable molecule as the conjugation moiety. Prodrugs having a nonpolar lipid as the conjugation moiety are only one class of the prodrugs described herein that may be successfully with a lipid-based complexation agent, including complexation agents that are also nonpolar lipids. A nonpolar lipid is a hydrophobic molecule that is solid at temperatures between 27 to 50 degrees C., containing ketoacyl and isoprene groups inclusive but not restricted to fatty acids, glycerolipids, glycerophospholipids, sphingolipids, saccharolipids, polyketides (derived from condensation of ketoacyl subunits), sterol lipids and prenol lipids (derived from condensation of isoprene subunits).

One specific example of H-d-Arg-DMT-Lys-Phe(—O)—R includes H-d-Arg-DMT-Lys-Phe(—O)-stearyl (depicted in FIG. 27A), wherein H-d-Arg-DMT-Lys-Phe is linked via ester bond to stearyl alcohol, one member from the group of long-chain saturated fatty alcohols. On cleavage of the ester bond, the prodrug H-d-Arg-DMT-Lys-Phe(—O)-stearyl releases the native MTT. To demonstrate this experimentally, H-d-Arg-DMT-Lys-Phe(—O)-stearyl was incubated at 37° C. in vitro with carboxyesterase (0.1 μg/mL), to simulate the ocular physiologic environment and the type of esterase that is readily abundant therein, within the vitreous. Incubation of H-d-Arg-DMT-Lys-Phe(—O)-stearyl with carboxyesterase produced rapid cleavage of the prodrug ester bond, releasing H-d-Arg-DMT-Lys-Phe, as evident by high performance liquid chromatography (HPLC) analysis and quantification of H-d-Arg-DMT-Lys-Phe and H-d-Arg-DMT-Lys-Phe(—O)-stearyl prodrug in solution (FIG. 28B). Upon addition of H-d-Arg-DMT-Lys-Phe(—O)-stearyl prodrug to phosphate-buffered saline solution at 37° C. without esterase, the ester bond of the H-d-Arg-DMT-Lys-Phe(—O)-stearyl prodrug cleaves more slowly (˜36 hours) by hydrolysis (FIG. 28C). Thus, in ocular physiologic system, the covalent bond of the prodrug linking MTT to inactive conjugation is readily cleaved either by enzymatic cleavage or more slowly by hydrolysis, releasing the active MTT.

Further, upon cleavage of the covalent bond of the drug substance, the API, the native MTT peptide, retains bioactivity for treatment of mitochondrial dysfunction. For example, as depicted in FIGS. 29A-29C, in an in vitro cell culture model of dry AMD, H-d-Arg-DMT-Lys-Phe(—O)-stearyl (5 μM) was added to RPE cells (which possess endogenous esterases) with mitochondrial dysfunction induced by exposure to hydroquinone (HQ). H-d-Arg-DMT-Lys-Phe(—O)-stearyl effectively reversed HQ-induced mitochondrial dysfunction in RPE cells (as depicted by cellular flavoprotein-autofluorescence), with efficacy equivalent to treatment with H-d-Arg-DMT-Lys-Phe native peptide (5 μM). H-d-Arg-DMT-Lys-Phe(—O)-stearyl was also preincubated with carboxyesterase (0.1 μg/mL) in separate media. Recovered media containing cleaved H-d-Arg-DMT-Lys-Phe (5 μM) was added to this RPE cellular model of mitochondrial dysfunction, and this was similarly effective and equipotent to H-d-Arg-DMT-Lys-Phe native peptide for the reversal of RPE mitochondrial dysfunction. Thus, these studies affirm that the active API that is cleaved from prodrug retains essential and unmodified bioactivity for the treatment of mitochondrial dysfunction.

In some instances, a conjugation moiety, which may be combine elements from two or more of these classes, may serve as as a multimeric linker moiety that is covalently linked to multiple molecules of the API to form dimers and/or multimers. Such linkers capable of generating dimers or multimers of mitochondria targeting peptides may be referred to as “multimerization domains.”

Prodrug with multimerization domain has formula (III):

(R′)ₙ—R (III)

wherein R is a linker or multimerization domain which is covalently linked to multiple API R′, to form dimers or multimers of the API and n is equal to 2 to about 100. Examples include PEG polymers, polyvinyl alcohol (PVA) polymers, or polypeptides, where the linker conjugation moiety R is covalently linked to two or more molecules of the API R′, to form dimers, trimers, multimers, etc. In some cases, the multimerization domains have alcohols, i.e., multiple “—OH” groups, to which the API units R′ are bound. In this setting, multiple API covalently linked (e.g., via ester or another dynamic covalent bond) to the multimerization domain may be referred to an API multimer.

One example of such a prodrug multimer is the mitochondrial-targeted tetrapeptide H-d-Arg-DMT-Lys-Phe linked to PVA compound, with the formula, where “n” is number comprising PVA polymer:

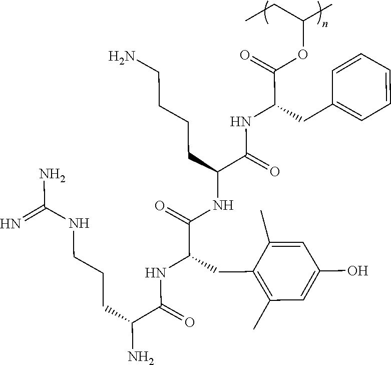

The dispersal medium of the drug substance multiphasic colloidal suspension is defined herein as a hydrophobic liquid into which drug substance and particulate complexation agents are admixed to form a stable multiphasic colloidal suspension.

The criteria that define a stable multiphasic colloidal suspension include uniform mixture and distribution of the drug substance-complex particulates without settling, separation, or dissociation of the particulates for the prespecified duration of the implant's lifetime, after exposure to an ocular physiologic environment in vitro (i.e., 37° C., buffered saline, vitreous enzymes, dilute serum) or in vivo when injected into the eye. The stability is also dependent on the relative percentage of drug substance-complex particulates to oil (weight to weight) and the size and mass of the particulates.

The methods and compositions described herein describe new previously unrecognized properties of certain oils that allow them to serve as effective dispersal medium. These include hydrophobicity, high starting viscosity, and other properties that allow it to form a stable multiphasic colloidal suspension when admixed with drug substance-complex particulates.

Four classes of oils that meet these criteria for dispersal medium include saturated fatty acid methyl esters, unsaturated fatty acid methyl esters, saturated fatty acid ethyl esters, or unsaturated fatty acid ethyl esters. A dispersal medium can be an individual oil from one of these classes or can be designed as a mixture of oils with different viscosity values that are specifically designed and admixed to achieve the desired goal of a stable colloidal suspension.

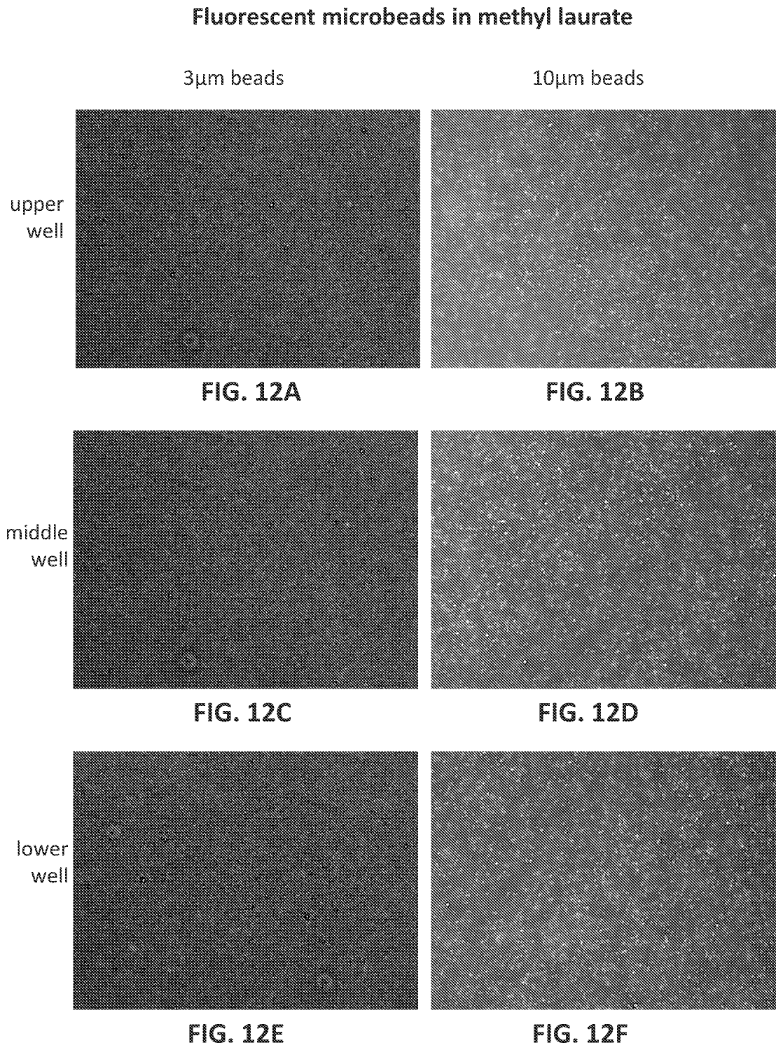

Saturated fatty acid methyl esters that may serve as dispersal medium include: methyl acetate, methyl propionate, methyl butyrate, methyl pentanoate, methyl hexanoate, methyl heptanoate, methyl octanoate, methyl nonanoate, methyl decanoate, methyl undecanoate, methyl dodecanoate (methyl laurate) (FIGS. 12A-12F), methyl tridecanoate, methyl tetradecanoate, methyl 9(Z)-tetradecenoate, methyl pentadecanoate, methyl hexadecanoate, methyl heptadecanoate, methyl octadecenoate, methyl nonadecanoate, methyl eicosanoate, methyl heneicosanoate, methyl docosanoate, methyl tricosanoate, and others.

Unsaturated fatty acid methyl esters that may serve as dispersal medium include: methyl 10-undecenoate, methyl 11-dodecenoate, methyl 12-tridecenoate, methyl 9(E)-tetradecenoate, methyl 10(Z)-pentadecenoate, methyl 10(E)-pentadecenoate, methyl 14-pentadecenoate, methyl 9(Z)-hexadecenoate, methyl 9(E)-hexadecenoate, methyl 6(Z)-hexadecenoate, methyl 7(Z))-hexadecenoate, methyl 11(Z)-hexadecenoate.

Saturated fatty acid ethyl esters that may serve as dispersal medium include: ethyl acetate, ethyl propionate, ethyl butyrate, ethyl pentanoate, ethyl hexanoate, ethyl heptanoate, ethyl octanoate, ethyl nonanoate, ethyl decanoate, ethyl undecanoate, ethyl dodecanoate (ethyl laurate), ethyl tridecanoate, ethyl tetradecanoate, ethyl 9(Z)-tetradecenoate, ethyl pentadecanoate, ethyl hexadecanoate, ethyl heptadecanoate, ethyl octadecenoate, ethyl nonadecanoate, ethyl eicosanoate, ethyl heneicosanoate, ethyl docosanoate, ethyl tricosanoate.

Unsaturated fatty acid ethyl esters that may serve as dispersal medium include: ethyl 10-undecenoate, ethyl 11-dodecenoate, ethyl 12-tridecenoate, ethyl 9(E)-tetradecenoate, ethyl 10(Z)-pentadecenoate, ethyl 10(E)-pentadecenoate, ethyl 14-pentadecenoate, ethyl 9(Z)-hexadecenoate, ethyl 9(E)-hexadecenoate, ethyl 6(Z)-hexadecenoate, ethyl 7(Z))-hexadecenoate, ethyl 11(Z)-hexadecenoate.

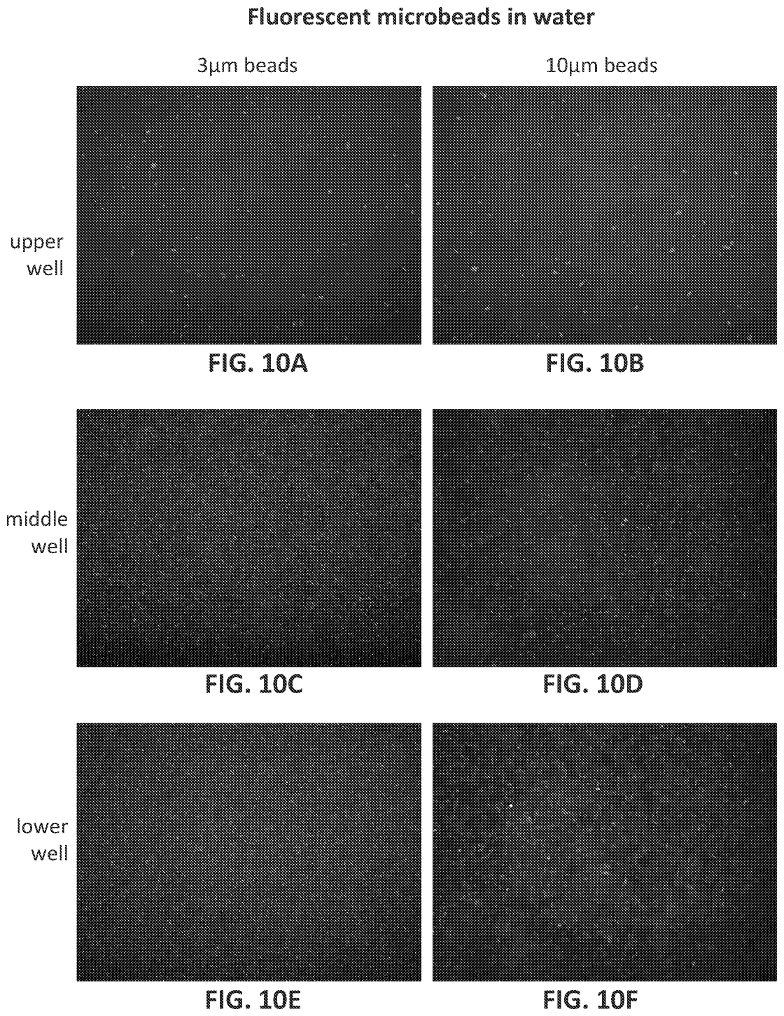

In contrast, certain other oils and viscous substances (FIGS. 10A-10F, 11A-11F, 13A-13F and 14A-14F) including silicone oil, viscous gelatin, and viscous proteoglycan fail to form a stable multiphasic colloidal suspension or rapidly decompensate when exposed to a physiologic ocular microenvironment (e.g., 37° C., buffered saline, vitreous enzymes, dilute serum) or in vivo when injected into the eye.

Complexation of drug substance to particulate complexation agents within the dispersal medium serves to limit the release of free drug substance into the dispersal medium. While the dispersal medium restricts access of water to the drug substance-complex particulates, free, unbound drug substance diffuses freely within the dispersal medium, and the dispersal medium does not retain the free, unbound drug, which can diffuse out of the multiphasic colloidal suspension.

Features of this complexation-based XRDDS clearly differentiate it from prior art of established XRDDS for ocular drug delivery.

A retention vehicle is a liquid or semi-solid substance in which the vehicle is chosen based on its physicochemical properties for interaction with drug substance in a manner that restricts or limits its release from the retention vehicle. Examples include but are not limited to oil-in-water emulsions, water-in-oil emulsions, viscous gelatin, hydrogels, and viscous chondroitin sulfate. A retention vehicle-based XRDDS does not have any requirement for stable dispersal of drug substance-complex particulates, and drug release is determined by the interaction of the retention vehicle with the drug substance, wherein the retention vehicle impedes or slows diffusion from the vehicle into the ocular physiologic environment. These properties differ from the preferred embodiment of drug substance in the multiphasic colloidal suspension XRDDS, wherein the drug substance-complex particulates are stably dispersed without settling or migration, and there is no requirement that the dispersal medium impedes or slows diffusion of the drug substance from the implant.

Carrier-based XRDDS represent a passive-release, bio-erodible formulation strategy. Carrier-based XRDDS are designed to physically trap drug substance in a specific carrier, but then the system must degrade via interactions with the tissue, not from mechanisms intrinsic within the XRDDS, in order to release free drug substance. In some embodiments, carrier formulations include a single device that compartmentalizes drug substance from the tissue. Examples include but are not limited to polymer-based rods or other shapes (drug trapped in a chemical substance extruded into rods or molded into different shapes), photopolymerizable or photo-crosslinked block polymer comprised of PLGA and other cross-linkable substrates in which drug substance is trapped within the polymer formulated into injectable viscous polymer or polymer-based rods or other shapes, polymer-based microparticles (which require chemical covalent crosslinking of small block polymers to trap drug), liposomes (phospholipid-in-water emulsion) sonicated to trap drug, all of which can be used to formulate drug substance. The common feature of all carrier-based systems is that the drug substance is trapped within the carrier material; as the carrier degrades, dissolves, or otherwise breaks down, free drug substance is released into the tissue. This may require a chemical or enzymatic reaction provided by the tissue microenvironment. In addition, the defects made in the carrier system during degradation allow access to water from the microenvironment, which further promotes release of the drug substance. Carrier-based systems differ from the multiphasic colloidal suspension, which has a hydrophobic dispersal medium and therefore repels water from entering the system. Further, in the multiphasic colloidal suspension, there is no requirement for the system to degrade via interactions with the tissue in order to release drug substance from the implant. The release kinetics are not determined by drug trapped in the multiphasic colloidal suspension.

Thus, the present multiphasic colloidal suspension is differentiated from previously conceived and designed systems such as retention vehicles and carrier based systems because it instead utilizes the chemistry of complexation systems specifically for sustained release drug delivery to the eye. The present system uses complexation of a drug onto one or more complexation agent(s) as a method to limit the unbound, free drug available for release and to regulate the kinetics of drug release into ocular tissue in a bioerodible modality or formulation.

The drug substance multiphasic colloidal suspension can be designed by specific process to meet a prespecified release rate and amount of drug substance, by varying the ratios and amounts of different drug substance-complex particulates, with different Kd and binding capacity (FIGS. 2A-2E). The property of Kd is a measure of avidity of a drug substance for a given complexation agent and is defined as the unbound-bound fraction of drug substance for a drug substance-complex particulate in a given dispersal medium. Specific Kd value can be measured by specified release assay, as described herein (FIGS. 15, 18, 20, 24). The property of binding capacity is defined as a maximal amount of drug that is bound to a known quantity of complexation agent.

Release of drug substance from the implant is determined in part by the unbound fraction within the dispersal medium, which is in turn determined in part by the Kd values and the binding capacity values for different drug substance-complex particulates. Knowledge of the Kd and binding capacity allows the choice of specific combinations of different prodrug-complexation agent particulates to regulate the unbound fraction of drug within the dispersal medium over time and to thus achieve a prespecified release kinetics profile (FIGS. 2A-2E and 9). The inclusion of more than one complexation agent in the multiphasic colloidal suspension can be used to regulate the unbound fraction of drug within the dispersal medium over time and thus the release kinetics of the system.

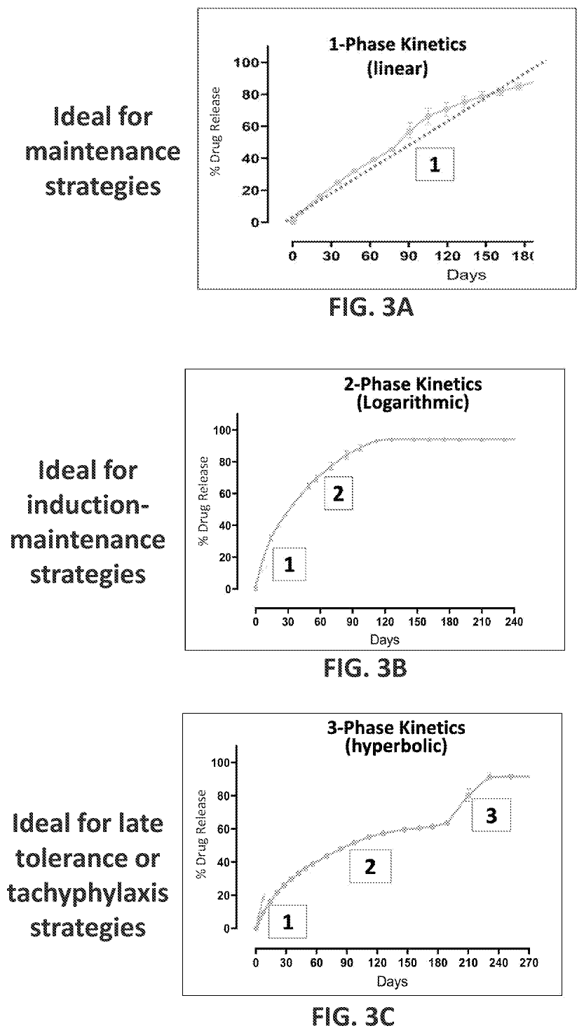

For example, the addition of drug substance-complex particulate with high binding capacity and high Kd, indicating low avidity of drug substance to the complex particulate, can be used to create a short-term increased rate of release, or initial burst. The addition of drug substance-complex particulates with high binding capacity and moderate Kd, indicating moderate avidity of drug substance to the complex particulate, can be used to create a long-term lower rate of release, to extend the duration of drug substance release from the implant. The combination of these two types of drug-substance particulates can be selected and admixed, in desired ratio and concentration, to achieve to create an implant with two-phase release kinetics of drug substance from the implants (FIG. 3B). An implant with this release kinetic profile would be useful for diseases that require a “loading” phase to treat and reverse established disease pathobiology, while the second “steady-state” phase would be effective for preventing onset of new or recurrent disease.

In another example, the addition of drug substance-complex particulate with high binding capacity and high Kd, indicating low avidity of drug substance to the complex particulate, can be used to create a short-term increased rate of release, or initial burst. The addition of drug substance-complex particulates with high binding capacity and moderate Kd, indicating moderate avidity of drug substance to the complex particulate, can be used to create a long-term lower rate of release, to extend the duration of drug substance release from the implant. The addition of drug substance-complex particulates with high binding capacity and low Kd, indicating high avidity of drug substance to the complex particulate, would release late an to create a late-term burst in the implant's lifetime. The combination of these three types of drug-substance particulates can be selected and admixed, in desired ratio and concentration, to achieve to create an implant with three-phase release kinetics of drug substance from the implants (FIG. 3C). An implant with this release kinetic profile would be useful for diseases that require a “loading” phase to treat and reverse established disease pathobiology, while the second “steady-state” phase would be effective for preventing onset of new or recurrent disease, and yet a third phase of “late burst” would be useful for diseases in which there is a loss of potency of the drug or diminished response of the target to the drug late in the life of the implant due to tachyphylaxis or other mechanisms that mediate downregulation of the drug target or diminished responsiveness to the drug substance.

In such examples, the combined effect for a combination of two or more drug substance-complex particulates incorporated into selected dispersal medium is release of the drug substance in two or more phases based on the integral of release rates from the individual drug-complexation agent particulate components that are incorporated and dispersed into the drug substance multiphasic colloidal suspension.

For example, FIG. 2 schematically illustrates the theoretical basis for design and construction of an extended release drug delivery system (XRDDS) implant producing a desired drug release kinetic profile for drug substance. Initially, a theoretical pharmacokinetic release curve (i.e., target release profile), in this depiction is linearized by log transformation, is designed representing the desired initial burst phase and subsequent steady-state release phase, to give desired daily release rate, total duration of delivery, and drug payload in the final formulation. An iterative process is the performed to identify specific member compounds from 2 or 3 difference classes of complexation agents, expected to form noncovalent interactions with the drug substance based on the physicochemical properties of the drug substance. Each drug substance-complex particulate is first combined at initial amount and ratio and drug-complex particulates are then admixed and incorporated within a proposed dispersal medium. The drug substance multiphasic colloidal suspension is put into “sink” conditions and two properties of the drug substance-complex particulate are measured: the Kd (unbound-bound fraction) at day 1, 3, 7, 14, and 21 (a good indicator of burst and general binding avidity); and the release kinetics (% of initial payload of drug released over time), where Kd1 corresponds to drug substance-complex 1 and Kd2 corresponds to drug substance-complex 2.

Curve fitting is then applied to the release curve of each drug-complex, and the linearized curves are then solved to determine the right combination (of 2 or 3 specific drug-complex pairs) that give release kinetics that meet the pre-determined desired composite target product profile.

As shown in FIG. 2, this “theoretically-designed” formulation containing the combination of 2 or 3 drug substance-complex particulates are then formulated and tested for actual release kinetics. If necessary, the ratios of the 2-3 selected drug substance-complex particulates can be re-adjusted iteratively until the final release kinetics meet the predetermined target product release profile.

In some instances, when the drug substance is a prodrug, for the second or third drug substance-complex particulate, the bioactive drug may be covalently linked to a different conjugation moiety to form a different prodrug structure and the complexation agent may be distinct from the first, with distinct Kd values, Kd1 and Kd2 of drug substance-complex particulates, based both on the differing conjugation moieties and the differing complexation agent between pairs.

Alternatively in some instances, when the drug substance is a prodrug, the conjugation moiety of the prodrug may differ between the first and second drug-complex pairs, but the complexation agent may be the same, with distinct Kd values, Kd1 and Kd2 of drug-complex pairs, based on the differing conjugation moieties between pairs.

The composite extended release drug delivery system is designed and customized for the physicochemical properties of the drug substance to regulate the release of free drug substance from the system into the tissue.

The actual release kinetics of achieved by the drug substance multiphasic colloidal suspension in in vivo vitreous concentrations may meet or exceed EC₅₀ for an extended-release duration of 1 month or more. The EC₅₀ reflects the concentration of the drug substance that achieves 50% of the maximal response therapeutic effect, for the given mechanism of action of the drug substance.

In formulations of drug substance multiphasic colloidal suspension with two-phase release kinetics, the concentration of drug substance in the vitreous may exceed the reversal EC₅₀ (i.e., drug concentration required to achieve 50% of the maximal effect) during the initial burst phase and subsequently exceed the prevention EC₅₀ for the second (steady-state) phase, wherein prespecified release kinetics and desired duration of drug release were achieved by specific design and use of different drug substance-complex particulates in the multiphasic colloidal suspension as described herein.

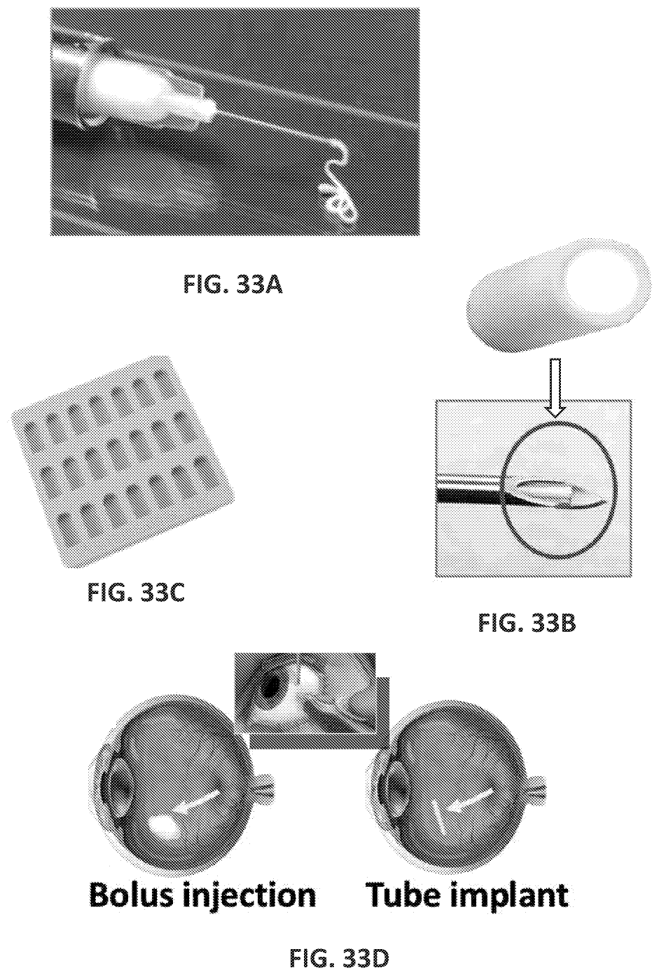

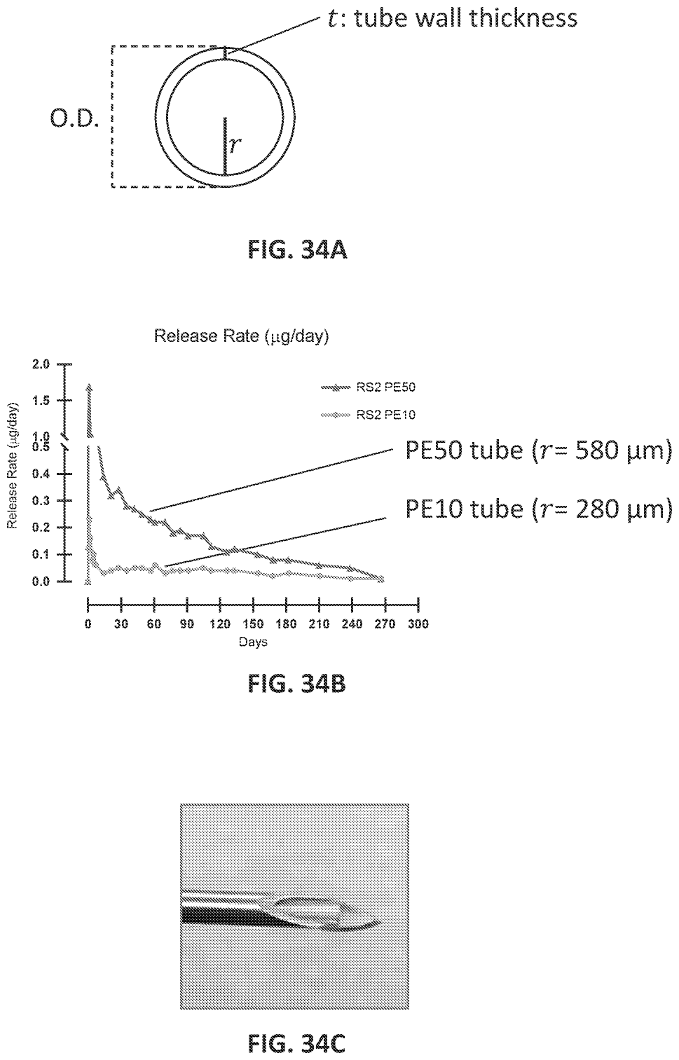

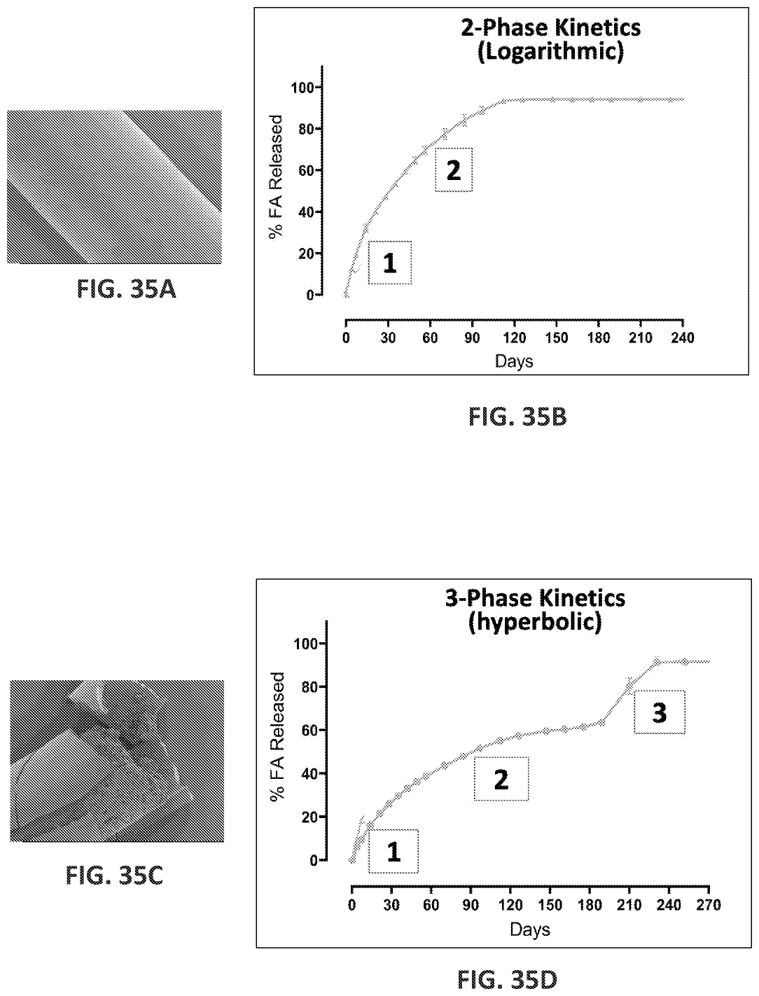

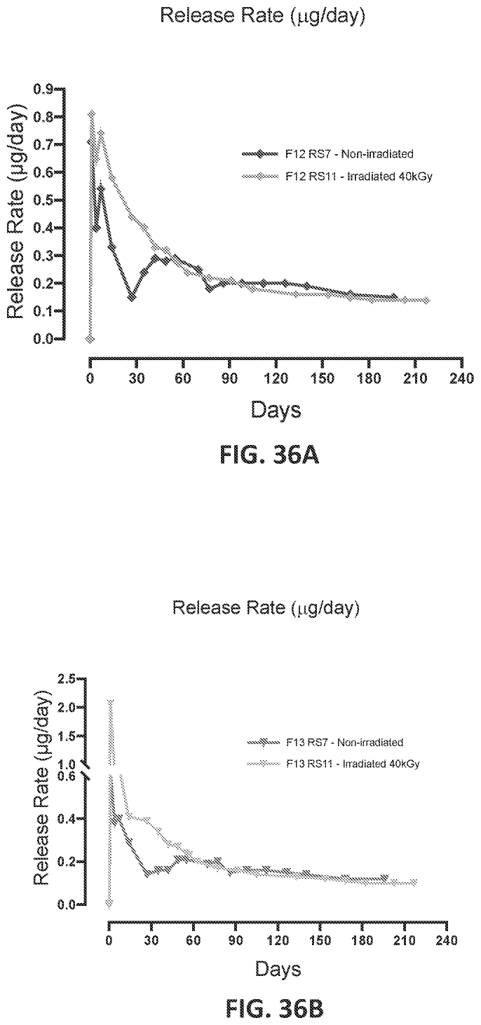

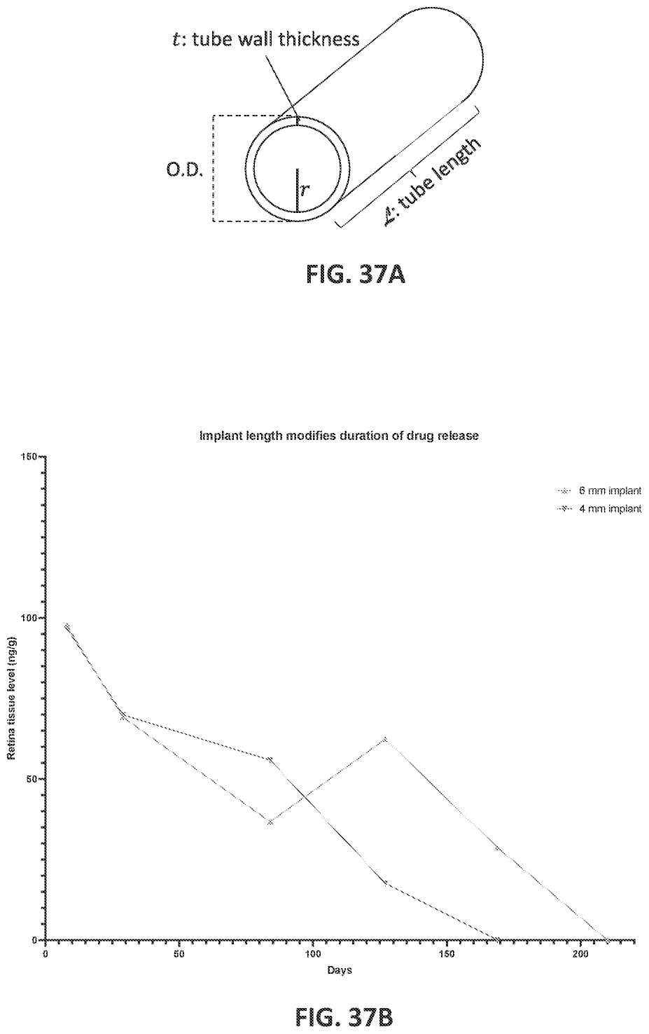

Formulations of the drug substance multiphasic colloidal suspension can be delivered as one of three different implant modalities, including a flowable bolus implant, an erodible or non-bioerodible tube implant filled with drug substance multiphasic colloidal suspension, or a solid mold of drug substance multiphasic colloidal suspension fashioned into specific size and shape, dried and hardened and configured for implantation (FIGS. 33A-33D). In some examples, the tube may itself be formed of the extended release drug delivery system. In other examples, the tube may be a comprised of a bio-erodible polymer that is compatible with ocular tissues (e.g., poly(lactic-co-glycolic acid, PLGA). In some examples, the tube may have one or both ends open for release of the mitochondrial targeted extended release compound.

Any of these formulations may be injected in and around the eye, i.e., into the vitreous humor, into the aqueous humor, into the suprachoroidal space, under the retina, under the conjunctiva, beneath Tenon's capsule, or into orbital tissue, to produce sustained release of therapeutic levels of drug substance within ocular tissues for desired duration (1 to 12 months), for the treatment of various diseases and disorders.

As a versatile extended release drug delivery system (XRDDS), the multiphasic colloidal suspension described herein can incorporate a variety of drug substances that directly form noncovalent complex with particulate complexation agents, as well as a variety of prodrugs, comprised of an active pharmaceutical ingredient (API) linked via cleavable covalent bond to a conjugation moiety, wherein the conjugation moiety of the prodrug forms noncovalent complex with particulate complexation agents. Specifically, the multiphasic colloidal suspension can incorporate various hydrophobic chemicals, hydrophilic chemicals, small polypeptides, proteins, aptamers, other nucleic acid drugs, and other chemical compounds.

Several examples are discussed herein, to demonstrate principles of complexation for drug delivery.

For example, a fluoresceinated, cationic small molecule was admixed with known quantities of selected individual complexation agents (FIGS. 4F, 5F, 6F, 7F). Different fluoresceinated small molecule-complex particulates were then admixed to an appropriate dispersal medium and visualized under fluorescent microscopy. Using this approach, fluoresceinated small molecule was observed to form fluoresceinated small molecule-complex particulates with several different complexation agents.

In another example, the tetrapeptide H-d-Arg-DMT-Lys-Phe was fluorescently labeled with fluorescein isothiocyanate (FITC) was admixed with known quantities of selected individual complexation agents (FIGS. 4B, 5B, 6B, 7B). Different fluoresceinated small molecule-complex particulates were then admixed to an appropriate dispersal medium and visualized under direct fluorescent microscopy. Using this approach, FITC-labeled H-d-Arg-DMT-Lys-Phe when admixed with different complexation agents (e.g., magnesium stearate, albumin), did not produce visible drug-complex particulates.

In another example, the same tetrapeptide H-d-Arg-DMT-Lys-Phe was linked by ester bond to stearyl alcohol, to form the prodrug H-d-Arg-DMT-Lys-Phe(O)-stearyl. The prodrug H-d-Arg-DMT-Lys-Phe(O)-stearyl 1 was fluorescently labeled with FITC and admixed with different complexation agents (FIGS. 4C, 5C, 6C, 7C). The resultant mixture was then visualized under direct fluorescence microscopy. Using this approach, FITC-labeled H-d-Arg-DMT-Lys-Phe(O)-stearyl (in which the tetrapeptide was labeled with FITC) was observed to form drug-complex particulates with several different complexation agents: magnesium stearate (as previously described, and as expected); albumin, a large, charged carrier protein; and cyclodextran, a large cyclic carbohydrate molecule. In contrast, FITC-labeled H-d-Arg-DMT-Lys-Phe(O)-stearyl was not observed to consistently form drug-complex particulates with silica microbeads (FIG. 8C), indicating the process of complexation and drug-complex particulate formation is highly dependent on favorable noncovalent interaction between drug and complexation agent.

Since only H-d-Arg-DMT-Lys-Phe(O)-stearyl prodrug with conjugation moiety formed drug-complex particulates, it is inferred that complex formation was mediated by the conjugation moiety of the prodrug. To assess this, FITC-labeled H-d-Arg-DMT-Lys-Phe(O)-stearyl (in which the tetrapeptide was labeled with FITC) that had been admixed with complexation agent was treated with an aqueous solution of carboxyesterase (0.1 μg/mL) to hydrolyze the ester bond of the prodrug, releasing the fluorescent peptide (FIGS. 4D, 5D, 6D, 7D). Complexed particulates were no longer fluorescently labeled by microscopy, affirming that complexation of the prodrug is specifically mediated by its conjugation moiety and validating the concept of using a prodrug with compatible conjugation moiety to mediate complex formation.

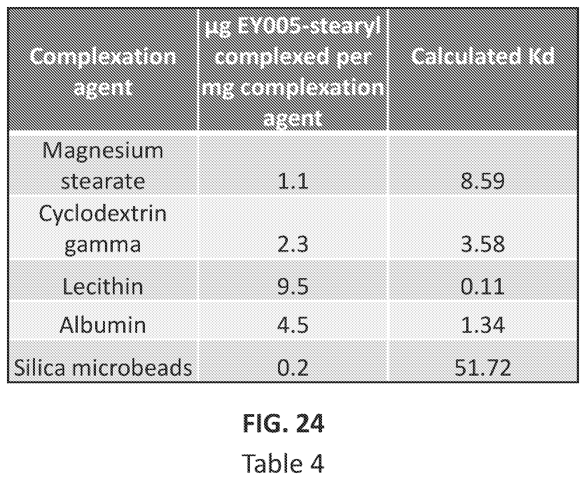

Further, as described herein, formation of drug-complex particulates in which the complexation agent has high avidity for the drug can be quantified and verified experimentally. For example, the prodrug H-d-Arg-DMT-Lys-Phe(O)-stearyl was admixed with known quantities of selected individual complexation agents (FIG. 24). The H-d-Arg-DMT-Lys-Phe(O)-stearyl-complexation agent mixture was then added to an appropriate dispersal medium (in this case, methyl laurate), and centrifuged to “pull down” or separate H-d-Arg-DMT-Lys-Phe(O)-stearyl bound to complexation agent from unbound prodrug present in the dispersal medium. HPLC analysis of pulled down particulates and dispersal medium from H-d-Arg-DMT-Lys-Phe(O)-stearyl content determined the fraction of prodrug that is bound to the complexation agent and calculation of the Kd value, the unbound to bound coefficient, and binding capacity for the prodrug-complexation agent particulate. Using this type of assay, Kd values and binding capacity can be generated for specific prodrug-complexation agent pairs in a selected dispersal medium (see FIG. 24).

Thus, in some examples, the formation of a prodrug substantially alters the physicochemical properties of the API to enable complexation and optimize its compatibility for formulation in the multiphasic colloidal suspension. The API H-d-Arg-DMT-Lys-Phe is highly hydrophilic, and as noted above, did not produce visible drug-complex particulates on admixture with complexation agents. Linkage via ester bond to stearyl alcohol produced the prodrug H-d-Arg-DMT-Lys-Phe(O)-stearyl, is highly hydrophobic as compared to the unmodified API. Further, the high avidity interaction between the hydrophobic, long-chain fatty alcohol of the conjugation moiety of this MTT-prodrug and particulate complexation agents serves to bind the MTT-prodrug and limits the free, unbound MTT-prodrug that is available for release from the dispersal medium in which the MTT-prodrug-complex particulates are dispersed (FIG. 25B).

Another specific example of prodrugs includes H-d-Arg-DMT-Lys-Phe(O)-tri-arginine (triArg) (depicted in FIG. 27C), wherein H-d-Arg-DMT-Lys-Phe is linked via ester bond to arginine trimer/tripeptide, a positively charged peptide conjugation moiety that readily forms noncovalent complex with negatively charged particulate complexation agents to form MTT-prodrug-complex particulates. The high avidity interaction between the positively conjugation moiety of this MTT-prodrug and the negative charge of the particulate complexation agent serves to bind this MTT-triArg prodrug and limits the free, unbound MTT-prodrug that is available for release from the dispersal medium in which the MTT-prodrug-complex particulate is dispersed.

Another specific example of prodrugs includes H-d-Arg-DMT-Lys-Phe(O)-tri-glutamate (triGlu) (depicted in FIG. 27B), wherein H-d-Arg-DMT-Lys-Phe is linked via ester bond to glutamate trimer/tripeptide, a negatively charged peptide conjugation moiety that readily forms noncovalent complex with positively charged particulate complexation agents to form MTT-prodrug-complex particulates. The high avidity interaction between the negatively charged conjugation moiety of this MTT-prodrug and the positive charge of the particulate complexation agent serves to bind MTT-triGlu prodrug and limits the free, unbound MTT-prodrug that is available for release from the dispersal medium in which the MTT-prodrug-complex particulate is dispersed.

In examples in which the conjugation moiety of EY005-prodrug is a pegylated peptide, such as EY005-polyethylene glycol (PEG) (FIG. 27D), the complexation agent may form noncovalent interactions with the PEG or PEGylated conjugation moiety based on its size and charge.

Several examples are discussed herein, to specifically identify and differentiate substances that can (and substances that cannot) serve as dispersal medium.

Herein, a dispersal medium is defined as a hydrophobic, viscous oil that when admixed with drug substance-complex particulates, can form a stable multiphasic colloidal suspension, which is formed into an implant for administration in or around the eye. Herein, colloidal indicates that the particulates are uniformly dispersed and stable indicates that the particulates remain dispersed without settling or migration for the duration of the implant's intended lifetime.

To better understand these properties and identify liquid substances that could serve as dispersal medium, fluorescent particulate beads of two different sizes, 3 μm (micrometer, or micron) and 10 μm, were used as surrogates for drug substance-complex particulates (to facilitate visualization and imaging of particulates). These fluorescent particulate beads were suspended in various liquids in small shallow cylindrical wells, which were then assessed by confocal fluorescent microscopy to assess the distribution of the particulate beads and to ascertain, via the confocal functionality that assesses various depths of the liquid, whether any settling of fluorescent bead particulates occurred.

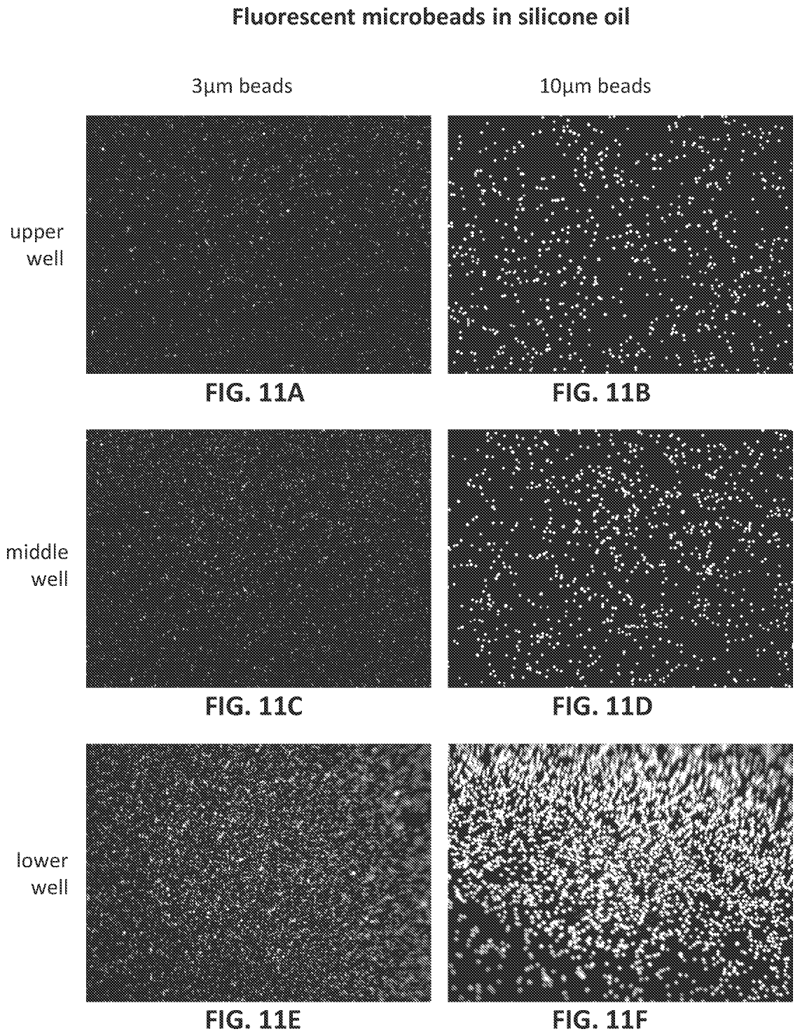

For example, when fluorescent particulate beads were admixed in water (FIGS. 10A-10F) and in silicone oil (FIGS. 11A-11F), they demonstrated substantially higher number and density of particulate beads in the bottom levels of the fluid, and relatively much fewer beads in the upper portion of the liquid. Thus, water and silicone oil did not uniformly disperse particulates, and no colloidal suspension was formed.

In another example, fluorescent particulate beads were admixed in the fatty acid methyl ester (FIGS. 12A-12F). Confocal microscopy demonstrated uniform distribution of the particulates regardless of depth of the liquid, indicating that methyl laurate uniformly dispersed particulates, forming a multiphasic colloidal suspension. Examination of this suspension in contact with an ocular physiologic environment (which contains enzymes and proteins typically contained in ocular tissues) over time, at 1 day, 1 week, and 1 month, demonstrated the stability of uniform distribution of particulates without migration within the colloidal suspension.

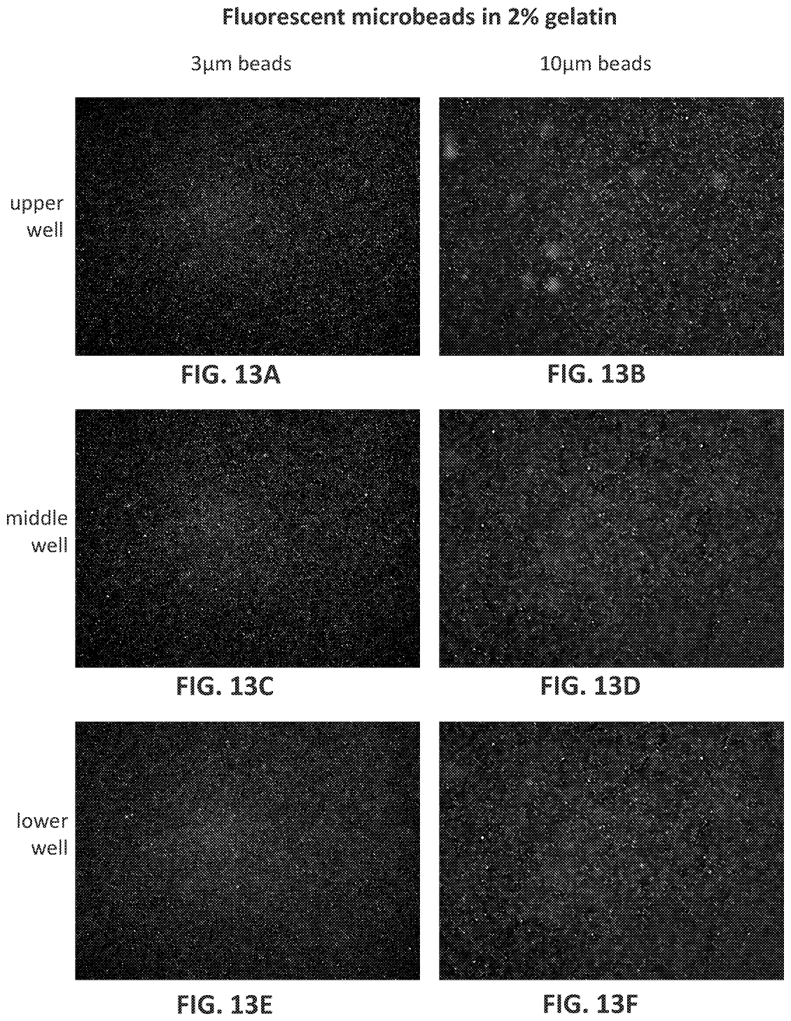

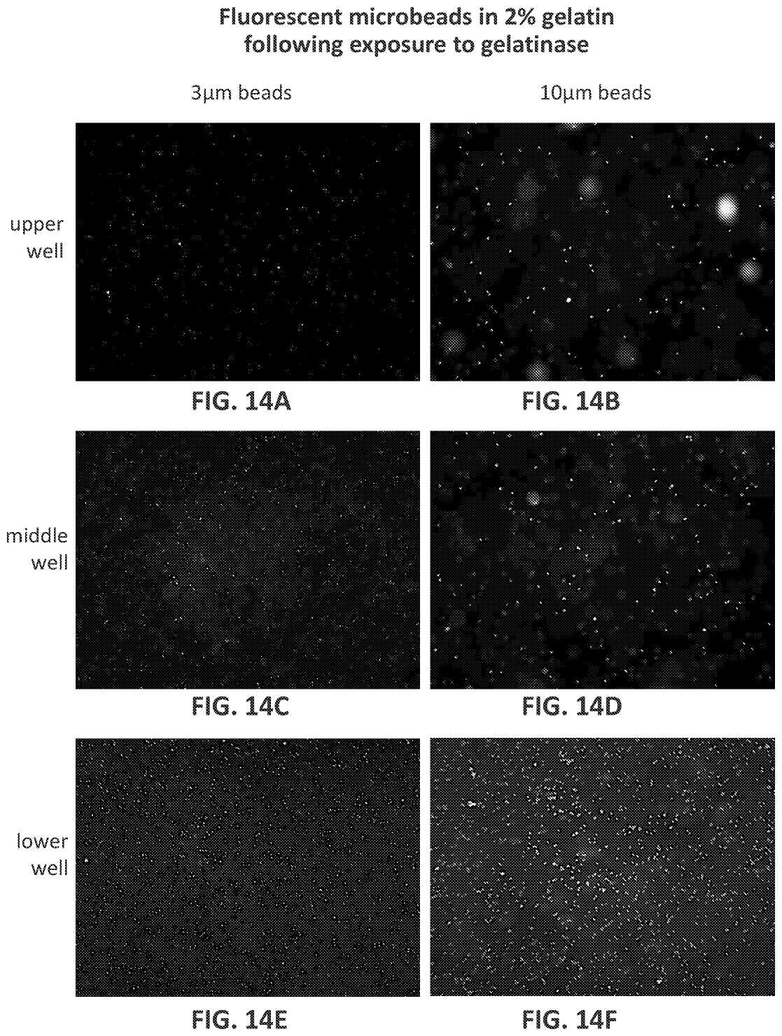

In another example, fluorescent particulate beads were admixed in 2% gelatin (FIGS. 13A-13F). Confocal microscopy demonstrated uniform distribution of the particulates regardless of depth of the liquid, indicating that 2% gelatin uniformly dispersed particulates, forming a multiphasic colloidal suspension. However, following placement into an in vitro ocular physiologic environment (which contains enzymes and proteins typically contained in ocular tissues) (FIGS. 14A-14F), the distribution of particulates within the suspension did not remain stable over time; the particulates migrated and as the 2% gelatin eroded and broke down.

Several examples are discussed herein, to demonstrate proof-of-concept for formulation and sustained release of various drug substances in the multiphasic colloidal suspension.

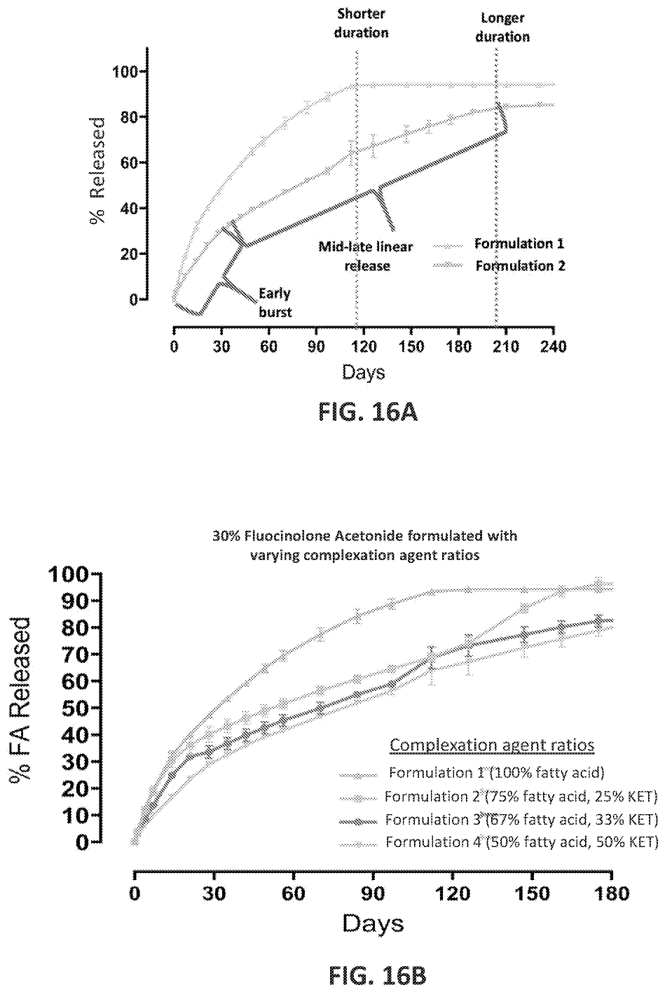

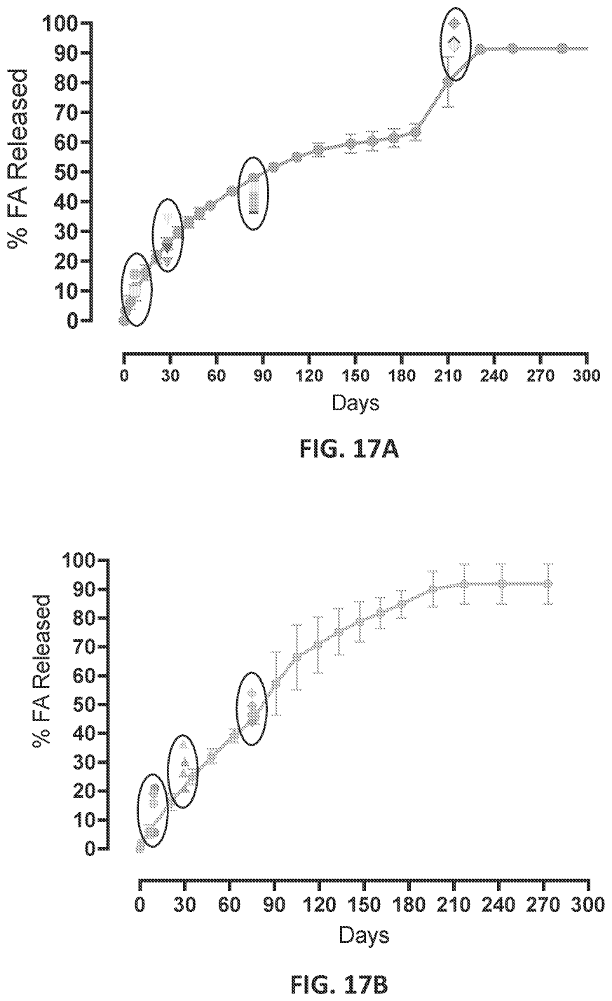

For example, formulations of the hydrophobic small molecule fluocinolone acetonide (FA) in the multiphasic colloidal suspension were developed. FA was admixed with different particulate complexation agents, to form various FA-complex particulate formulations (FIGS. 15, 16A-16B and 17-17B). Properties of Kd and binding capacity were calculated for each FA-complex particulate, in different dispersal media (data shown for dispersal medium of methyl laurate). A two-phase kinetic release profile was desired in this example. Based on this, complexation agents of magnesium stearate and tocopherol were selected for incorporation, along with FA, into dispersal medium of methyl laurate, in specific ratio and concentration to achieve a two-phase release for a bio-erodible tube formulation of FA multiphasic colloidal suspension. Formulations were iteratively refined by adjusting the ratio of FA-complexation agent particulates for a given payload of drug, to achieve approximately six-month duration of release, with an initial burst phase release followed by steady-state release.

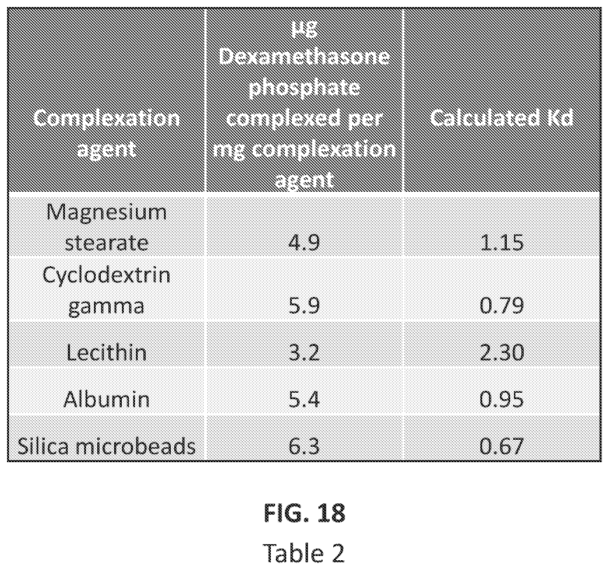

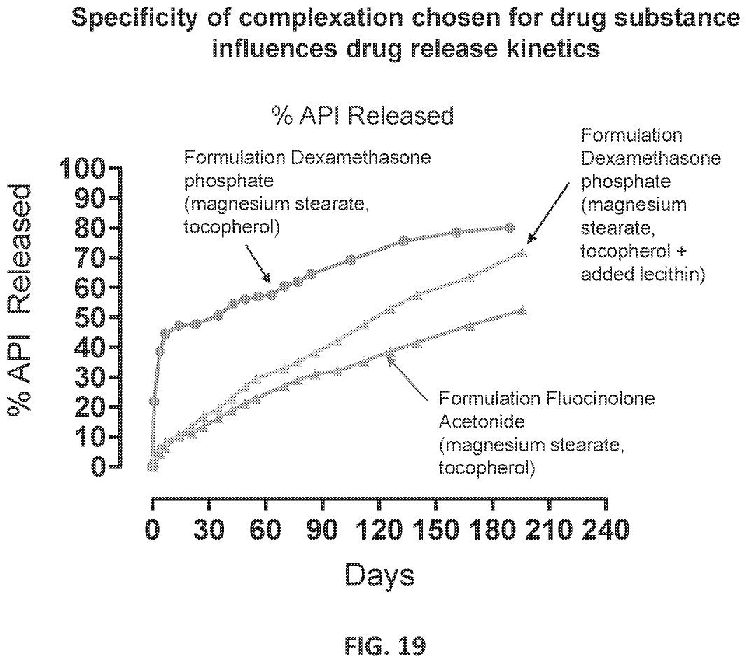

In another example, formulations of the hydrophilic small molecule dexamethasone phosphate (DexPh) in the multiphasic colloidal suspension were developed (FIGS. 18-19). To understand how physicochemical properties of the drug substance influence interactions with complexation properties, DexPh was admixed with same particulate complexation agents, magnesium stearate and tocopherol, and dispersal medium, chosen for the hydrophobic small molecule, FA. Formulation of DexPh demonstrated a rapid and excessive release, or “dump” of DexPh. The addition of a different complexation agent, with reduction in ratios of other complexation agents, for a given payload, altered the kinetic release profile to minimize dump of DexPh and provide more desirable sustained release profile, demonstrating the importance of selecting complexation agents on the basis of their favorable noncovalent complex formation with specific drug substance of interest.

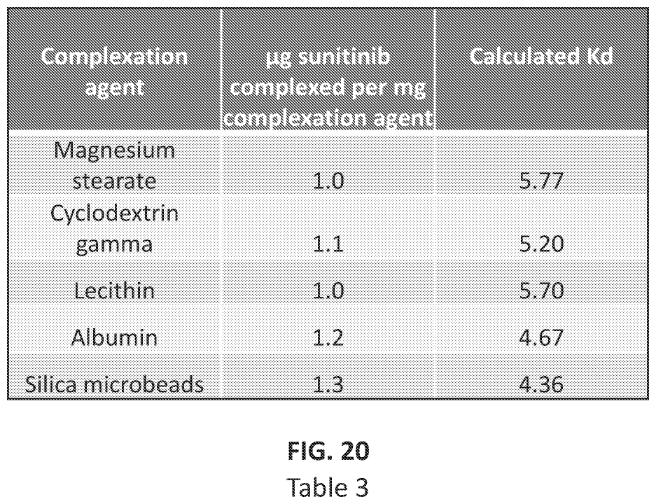

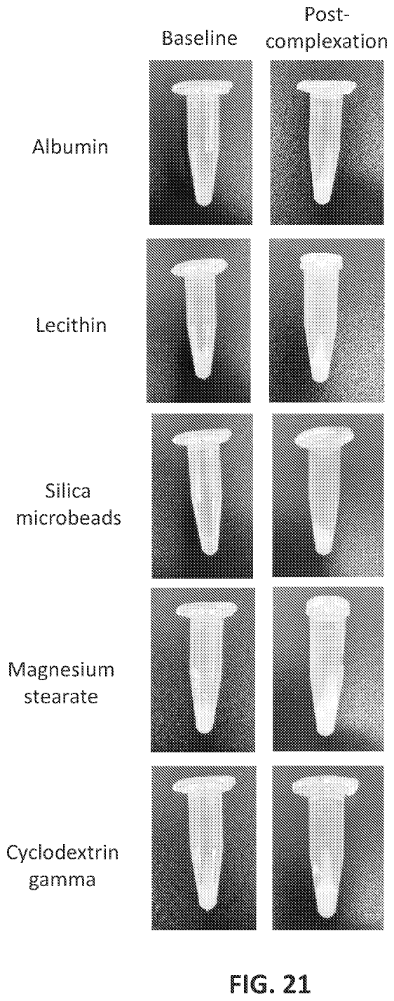

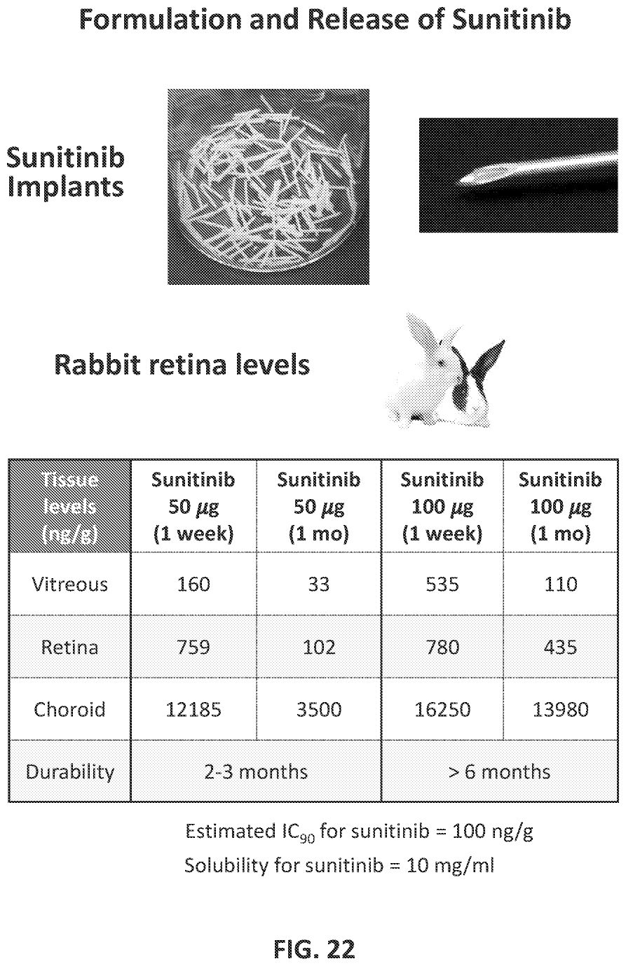

In another example, formulations of the hydrophilic small molecule sunitinib malate in the multiphasic colloidal suspension were developed. Sunitinib was admixed with different particulate complexation agents, to form various sunitinib-complex particulate formulations (FIGS. 20-22). Complexation of sunitinib to selected complexation agents was visually confirmed by admixture and pulldown of sunitinib-complex particulates, which was confirmed since by the yellow-orange color of particulates (sunitinib has orange coloration). Formulations of sunitinib with two-phase release kinetics in a bio-erodible tube formulation of sunitinib multiphasic colloidal suspension were designed and manufactured. Pharmacokinetics demonstrated two-phase release with high initial burst, followed by steady-state release. Alteration of the ratio of sunitinib-complex particulates reduced the amount of drug released during initial burst and lowered the steady state release.

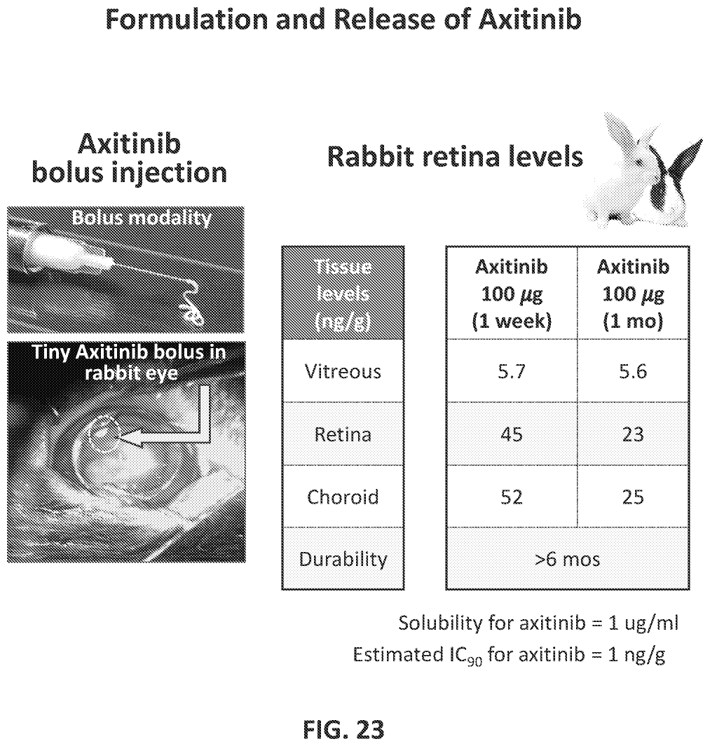

For example, formulations of the hydrophobic small molecule axitinib in the multiphasic colloidal suspension were developed. Axitinib was admixed with different particulate complexation agents, to form various axitinib-complex particulate formulations (FIG. 23). A formulation with single-phase kinetic release profile was desired in this example. Based on this, axitinib was admixed with complexation agent with high binding capacity and low Kd (indicating high affinity) in selected dispersal medium formulated as a bolus implant that produced a slow release formulation, with detectable drug in tissue.

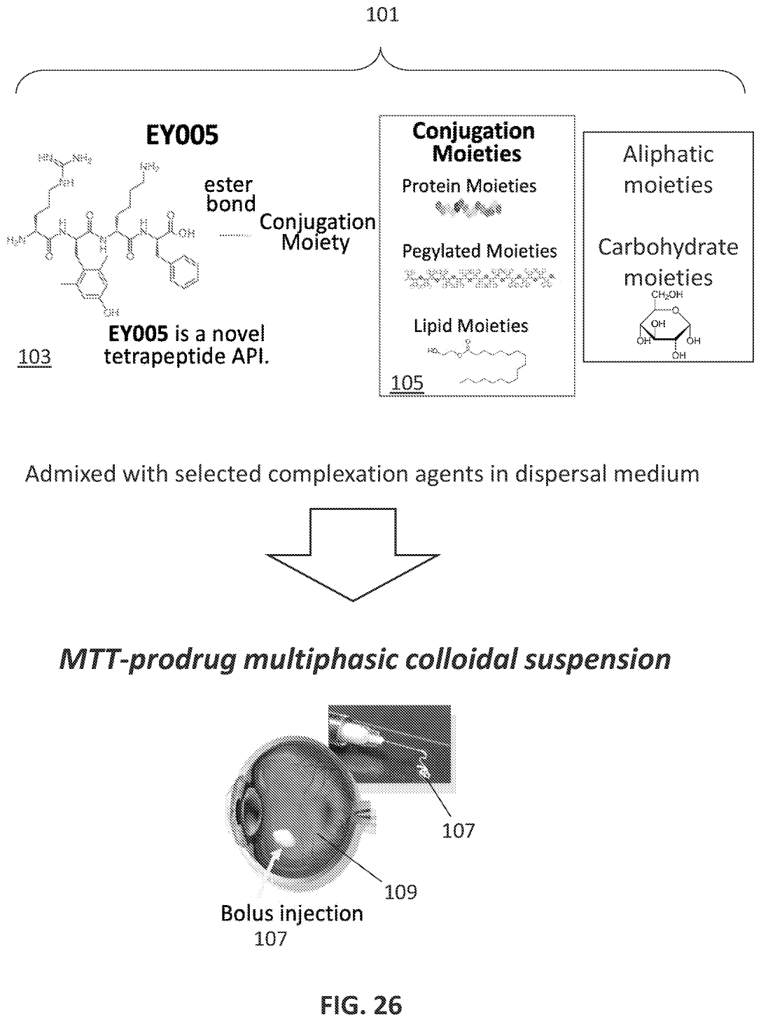

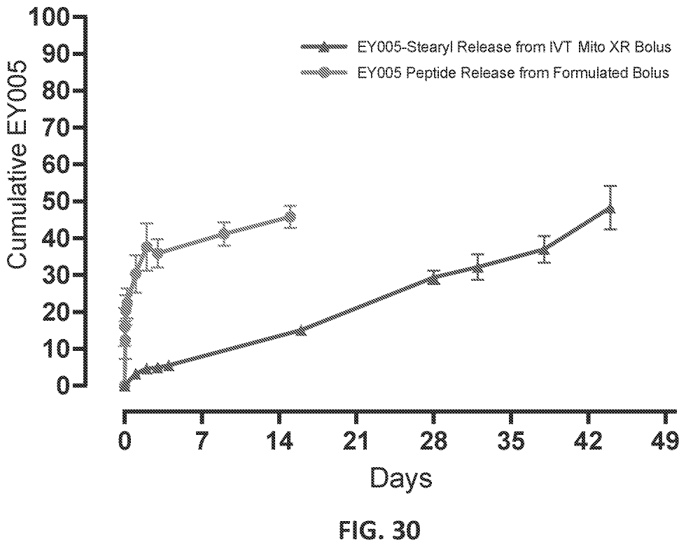

In another example, formulations of prodrug H-d-Arg-DMT-Lys-Phe(O)-stearyl in the multiphasic colloidal suspension were developed (FIGS. 24-32). In in vitro kinetics studies, pilot formulation of prodrug multiphasic colloidal suspension as a bolus implant achieved zero-order (i.e., linear) kinetics of release, achieving the desired durability of drug release of three months, with prodrug present in the dispersal medium and free API present in the in vitro physiologic environment following release of the prodrug from the multiphasic colloidal suspension (see FIG. 30).

In in vitro efficacy studies, bolus implant of prodrug multiphasic colloidal suspension was added to RPE cell culture model with endogenous esterases (FIGS. 31A-31D). Cell culture data demonstrated restoration of cytoskeleton, with ˜80% improvement at 21-day timepoint (FIG. 31D) in association with reversal of cellular mitochondrial dysfunction. This data affirms that prodrug, admixed with complexation agents and incorporated into a dispersal medium to form a stable multiphasic colloidal suspension, can produce sustained release of prodrug at predictable therapeutic levels, and the API remains bioactive upon cleavage of the MTT-prodrug in the surrounding in vitro physiologic environment.

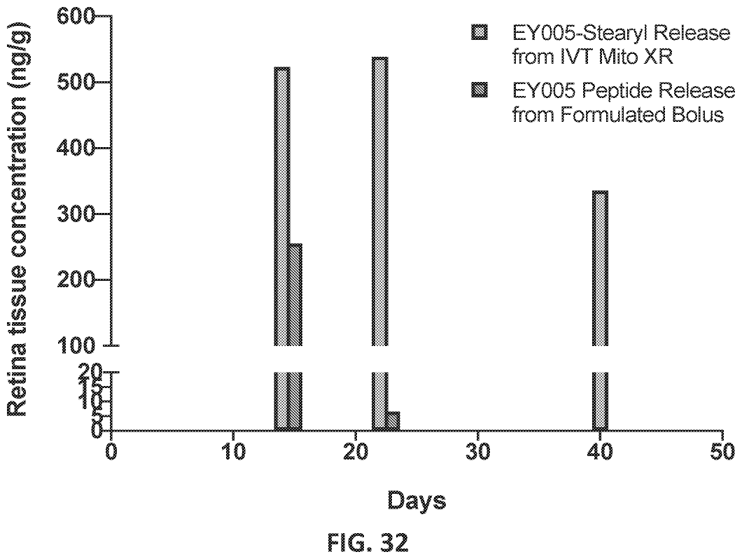

In in vivo kinetics studies, using LC/MS analysis, high retina levels (>300 ng/g) of MTT-prodrug were sustained through 6 weeks for intravitreal injection of a bolus implant of prodrug multiphasic colloidal suspension (H-d-Arg-DMT-Lys-Phe(O)-stearyl payload 1 mg) in rabbit eyes (FIG. 32), affirming good in vivo-in vitro correlation for release of prodrug. Recovered bolus had ˜50% residual payload, indicating the bolus implant of prodrug achieves the desired ˜90 day release kinetics of the implant, given zero-order release kinetics.

The relation Cₛₛ=Release rate/Clearance and the half-life (t_{1/2}) can be utilized to calculate the approximate desired daily release rate and drug payload of the extended release drug delivery system implant.

Further, incorporation of bioactive tetrapeptide API (without prodrug) with the same complexation agent and into the same dispersal medium produced excessive release, or “dump” of the bioactive API in vitro (FIG. 30) Additionally, the multiphasic colloidal suspension bolus formulation of native API administered into the vitreous did not produce detectable tissue levels beyond 21 days (FIG. 32), indicating excessive release of the native API in vivo as well. Moreover, no residual drug in the recovered bolus, consistent with excessive drug release or “dumping.” Thus, the incorporation of the native unmodified API into the multiphasic colloidal suspension is insufficient to produce sustained release and fails to achieve specifications of an extended release drug delivery system. Importantly, these data affirm and underscore the necessity for the prodrug construct and the specific interaction between prodrug conjugation moiety and complexation agent to form drug substance-complex particulates, for some APIs that do not otherwise form complexes, in order to achieve controlled, durable release of the active API from the multiphasic colloidal suspension XRDDS.

Formulations of the drug substance multiphasic colloidal suspension termed the implant, can be administered in and around the eye, i.e., into the vitreous humor (FIG. 33D), into the aqueous humor, into the suprachoroidal space, under the retina, under the conjunctiva, beneath Tenon's capsule, into orbital tissue, to produce sustained release of therapeutic levels of drug substance within ocular tissues for desired duration (1 to 12 months).

Formulations of the drug substance multiphasic colloidal suspension may be used to prevent onset or slow progression, modify disease pathobiology, prevent vision loss or improve vision, or prevent onset or improve other destructive or degenerative aspects of ocular conditions and diseases, including dry age-related macular degeneration (AMD), wet AMD, diabetic macular edema (DME), retinal vein occlusion (RVO), and inherited retinal degeneration (IRD), retinal degeneration, traumatic injury, ischemic vasculopathy, acquired or hereditary optic neuropathy, glaucoma, endophthalmitis, retinitis, uveitis, inflammatory diseases of the retina and uveal tract, Fuch's corneal dystrophy, corneal edema, ocular surface disease, dry eye disease, diseases of the conjunctiva, diseases of the periocular tissue, and diseases of the orbit.

The method may be used in conjunction with other treatment modalities including inhibition of vascular endothelial growth factor, complement inhibition, or administration of anti-inflammatory drugs such as corticosteroids.

All of the methods and apparatuses described herein, in any combination, are herein contemplated and can be used to achieve the benefits as described herein.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-31. (canceled)

32. A composition of a multiphasic colloidal suspension comprising a drug substance and one or more complexation agents, admixed in a dispersal medium having a release profile of one or more phases of drug release, wherein the one or more complexation agents is formulated as an irregular-shaped particulate that forms drug substance-complex particulates by noncovalent, reversible binding to the drug substance, and is one of: a fatty acid, an organic compound that can form a keto-enol tautomer, a charged phospholipid, a charged protein, a ribonucleic acid, and a polysaccharide; further wherein the dispersal medium is a hydrophobic liquid oil comprising at least one of: saturated fatty acid methyl esters, unsaturated fatty acid methyl esters, saturated fatty acid ethyl esters, unsaturated fatty acid ethyl esters.

33. The composition of claim 32, wherein the one or more complexation agents is a fatty acid comprising: a carboxylic acid with an aliphatic chain with chemical formula of CH3(CH2)ₙCOOH where n is equal to between 4 and 30, which is either saturated or unsaturated, and is a salt or an ester, and which includes one or more of: magnesium palmitate, magnesium stearate, calcium palmitate, calcium stearate.

34. The composition of claim 32, wherein the one or more complexation agents is a particulate complexation agent comprising an organic compound that can form a keto-enol tautomer and is capable of undergoing chemical equilibrium between a keto form consisting of a ketone or an aldehyde, and an enol form and includes one or more of: a phenol compound, a tocopherol compound, a quinone compound, a ribonucleic acid compound.

35. The composition of claim 32, wherein the one or more complexation agents is a particulate complexation agent that is a charged phospholipid and includes one or more of: an anionic phospholipid, lecithin, phosphatidylcholine, phosphatidylethanolamine, phosphatidylserine, sphingomyelin, a synthetic phospholipid with a positive charge, and DLin-MC3-DMA.

36. The composition of claim 32, wherein the one or more complexation agents is a particulate complexation agent that is a charged protein that is either positive or negative and includes one or more of: albumin, a synthetic polypeptide, a plasma protein, alpha2-macroglobulin, fibrin, and collagen.

37. The composition of claim 32, wherein the one or more complexation agents is a particulate complexation agent that is one or more of: a ribonucleic acid, a biopolymer macromolecule comprising nucleotides comprising a 5-carbon sugar, a phosphate group, and a nitrogenous base.

38. The composition of claim 32, wherein the one or more complexation agents is a particulate complexation agent that is a polysaccharide, comprising a long chain polymeric carbohydrates comprising monosaccharide units bound together by glycosidic linkages, and includes one or more of: a ringed polysaccharide molecule, a cyclodextrin, and a clathrate.

39. The composition of claim 32, wherein the drug substance forms noncovalent complexes with the particulate complexation agent, and comprises one of: a small molecule, a small polypeptide, a protein, an aptamer, a nucleic acid drug, a hydrophobic chemical, and a hydrophilic chemical.

40. The composition of claim 32, wherein the drug substance is a prodrug of formula (I): 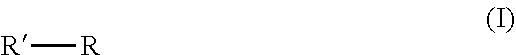
where R′ is any active pharmaceutical ingredient (API) that is covalently linked via cleavable bond to R, a conjugation moiety that forms noncovalent complexes with one of five classes of complexation agents, and the covalent bond linking R′ and R may be removed by enzymatic cleavage, catalysis, hydrolysis, or other reaction to yield free API R′ and conjugation moiety R, where R is selected from:
a C4-C30 lipid moiety (fatty acid or fatty alcohol),
an C4-C30 straight-chain or branched aliphatic moiety,
a 2-mer to 30-mer peptide moiety,
a pegylated moiety, or
a carbohydrate moiety.

41. The composition of claim 40, wherein the cleavable covalent bond comprises one of: an ester bond, a hydrazone bond, an imine bond, a disulfide bond, a thioester bond, a thioether bond, a phosphate ester bond, a phosphonate ester bond, a boronate ester bond, an amide bond, a carbamate ester bond, a carboxylate ester bond, and a carbonate ester bond.

42. The composition of claim 40, wherein the conjugation moiety, R, is fatty alcohol, with or without a preceding linker moiety, that includes one or more of: tert-butyl alcohol, tert-amyl alcohol, 3-methyl-3-pentanol, 1-heptanol (enanthic alcohol), 1-octanol (capryl alcohol), 1-nonanol (pelargonic alcohol), 1-decanol (decyl alcohol, capric alcohol), undecyl alcohol (1-undecanol, undecanol, hendecanol), dodecanol (1-dodecanol, lauryl alcohol), tridecyl alcohol (1-tridecanol, tridecanol, isotridecanol), 1-tetradecanol (myristyl alcohol), pentadecyl alcohol (1-pentadecanol, pentadecanol), 1-hexadecanol (cetyl alcohol), cis-9-hexadecen-1-ol (palmitoleyl alcohol), heptadecyl alcohol (1-n-heptadecanol, heptadecanol), 1-octadecanol (stearyl alcohol), 1-octadecenol (oleyl alcohol), 1-nonadecanol (nonadecyl alcohol), 1-eicosanol (arachidyl alcohol), 1-heneicosanol (heneicosyl alcohol), 1-docosanol (behenyl alcohol), cis-13-docosen-1-ol (erucyl alcohol), 1-tetracosanol (lignoceryl alcohol), 1-pentacosanol, 1-hexacosanol (ceryl alcohol), 1-heptacosanol, 1-octacosanol (montanyl alcohol, cluytyl alcohol), 1-nonacosanol, 1-triacontanol (myricyl alcohol, melissyl alcohol).

43. The composition of claim 40, wherein the conjugation moiety, R, is a fatty acid, with or without a preceding linker moiety, that comprises one or more of: Tetradecanoic acid, pentadecanoic acid, (9Z)-hexadecenoic acid, Hexadecanoic acid, Heptadecanoic acid, Octadecanoic acid, (9Z,12Z)-octadeca-9,12-dienoic acid, (9Z,12Z,15Z)-octadeca-9,12,15-trienoic acid, (6Z,9Z,12Z)-octadeca-6,9,12-trienoic acid, (5E,9E,12E)-octadeca-5,9,12-trienoic acid, (6Z,9Z,12Z,15Z)-octadeca-6,9,12,15-tetraenoic acid, (Z)-octadec-9-enoic acid, (11E)-octadec-11-enoic acid, (E)-octadec-9-enoic acid, nonadecanoic acid, and eicosanoic acid.

44. The composition of claim 40, wherein R is a 2-mer to about a 30-mer peptide moiety comprising natural or synthetic amino acids, which is one of: anionic, cationic, or neutral, with or without a preceding linker moiety, that includes one or more of: poly-glutamate, poly-aspartate, or a combination of glutamate and aspartate; poly-arginine, poly-lysine, poly-histidine, a combination of arginine and lysine, a combination of arginine and histidine, a combination of histidine and lysine, or a combination of arginine, histidine, and lysine; peptide moiety has one or more PEGylation sites for addition of polyethylene glycol (PEG) groups; peptide moiety has one or more sites for modification by addition of sugar or carbohydrate molecules, including glycosylation.

45. The composition of claim 40, wherein R is one of: a polyethylene glycol (PEG) polymer, a pegylated peptide, or pegylated succinate including PEG polymers of linear, branched, Y-shaped, or multi-arm geometries.

46. The composition of claim 40, wherein R is a carbohydrate moiety comprising a carbohydrate of 2 to 20 sugars, with or without a preceding linker moiety, comprising one or more of: glucose, galactose, lactose, mannose, ribose, fucose, N-acetylgalactosamine, N-acetylglucosamine, N-acetyleneuraminic acid, or an epimer or derivative of glucose, galactose, lactose, mannose, ribose, fucose, N-acetylgalactosamine, N-acetylglucosamine, and N-acetyleneuraminic acid.

47. The composition of claim 40, wherein R′ is an API, and R is a linker or multimerization domain which is covalently linked to multiple API to form dimers or multimers of the prodrug and n is equal to 2 to about 100, and R is one of: a PEG, a PEG polymer, polyvinyl alcohol (PVA), or peptide.

48. The composition of claim 32, wherein the dispersal medium is a liquid oil capable of forming multiphasic colloidal suspension, comprising a hydrophobic oil comprising at least one of: saturated fatty acid methyl esters, unsaturated fatty acid methyl esters, saturated fatty acid ethyl esters, unsaturated fatty acid ethyl esters.

49. The composition of claim 48, wherein the dispersal medium comprises a saturated fatty acid methyl ester comprising one or more of: methyl acetate, methyl propionate, methyl butyrate, methyl pentanoate, methyl hexanoate, methyl heptanoate, methyl octanoate, methyl nonanoate, methyl decanoate, methyl undecanoate, methyl dodecanoate (methyl laurate), methyl tridecanoate, methyl tetradecanoate, methyl 9(Z)-tetradecenoate, methyl pentadecanoate, methyl hexadecanoate, methyl heptadecanoate, methyl octadecenoate, methyl nonadecanoate, methyl eicosanoate, methyl heneicosanoate, methyl docosanoate, methyl tricosanoate, and others.

50. The composition of claim 48, wherein the dispersal medium comprises an unsaturated fatty acid methyl ester comprising one or more of: methyl 10-undecenoate, methyl 11-dodecenoate, methyl 12-tridecenoate, methyl 9(E)-tetradecenoate, methyl 10(Z)-pentadecenoate, methyl 10(E)-pentadecenoate, methyl 14-pentadecenoate, methyl 9(Z)-hexadecenoate, methyl 9(E)-hexadecenoate, methyl 6(Z)-hexadecenoate, methyl 7(Z))-hexadecenoate, methyl 11(Z)-hexadecenoate.

51. The composition of claim 48, wherein the dispersal medium comprises a saturated fatty acid ethyl ester comprising one or more of: ethyl acetate, ethyl propionate, ethyl butyrate, ethyl pentanoate, ethyl hexanoate, ethyl heptanoate, ethyl octanoate, ethyl nonanoate, ethyl decanoate, ethyl undecanoate, ethyl dodecanoate (ethyl laurate), ethyl tridecanoate, ethyl tetradecanoate, ethyl 9(Z)-tetradecenoate, ethyl pentadecanoate, ethyl hexadecanoate, ethyl heptadecanoate, ethyl octadecenoate, ethyl nonadecanoate, ethyl eicosanoate, ethyl heneicosanoate, ethyl docosanoate, ethyl tricosanoate.

52. The composition of claim 48, wherein the dispersal medium comprises an unsaturated fatty acid ethyl ester comprising one or more of: ethyl 10-undecenoate, ethyl 11-dodecenoate, ethyl 12-tridecenoate, ethyl 9(E)-tetradecenoate, ethyl 10(Z)-pentadecenoate, ethyl 10(E)-pentadecenoate, ethyl 14-pentadecenoate, ethyl 9(Z)-hexadecenoate, ethyl 9(E)-hexadecenoate, ethyl 6(Z)-hexadecenoate, ethyl 7(Z))-hexadecenoate, ethyl 11(Z)-hexadecenoate.

53. A composition of a multiphasic colloidal suspension comprising a drug substance and one or more complexation agents, admixed in a dispersal medium having a release profile of one or more phases of drug release, wherein the one or more complexation agents is formulated as an irregular-shaped particulate that forms drug substance-complex particulates by noncovalent, reversible binding to the drug substance, and is one of: a fatty acid, an organic compound that can form a keto-enol tautomer, a charged phospholipid, a charged protein, a ribonucleic acid, and a polysaccharide, further wherein the drug substance comprises one of: a small molecule, a small polypeptide, a protein, an aptamers, a nucleic acid drug, a hydrophobic chemical, and a hydrophilic chemical; further wherein the dispersal medium is a hydrophobic liquid oil comprising at least one of: saturated fatty acid methyl esters, unsaturated fatty acid methyl esters, saturated fatty acid ethyl esters, unsaturated fatty acid ethyl esters.

54. A method of treating a disorder and disease of the eye, by intravitreal or periocular injection of formulations of an extended release drug delivery system that produces high sustained retina and retinal pigment epithelium (RPE) tissue levels of active drug, the method comprising:
delivering a drug substance that is a prodrug combined with the extended-release drug delivery system into the subject's eye at a treatment start; and
cleaving, by action of an esterase or bioactive enzyme in the subject's eye, the prodrug to release the active pharmaceutical ingredient (API) of the prodrug into the eye during a first phase at a burst phase release rate; and
cleaving, by action of the esterase or bioactive enzyme, the prodrug to release the API into the eye during a second phase at a steady-state dose rate, wherein the burst phase rate is greater than the steady state release rate, further wherein the first phase extends from the treatment start for about 2-6 weeks and the subsequent phases extend from an end of the first phase for one or more months.
